SEQUENCE LISTING (12 sequences)

SEQ ID NO: 1 - protein, 210 aa, Homo sapiens
MWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 2 - protein, 130 aa, Homo sapiens
SGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

SEQ ID NO: 3 - protein, 80 aa, Artificial Sequence
  Description of Artificial Sequence Synthetic polypeptide
MWTLGRRAVAGLLASPSPAQAQTLTRVPRPAELAPLCGRRGLRTDIDATCTPRRASSNQRGLNQIWNVKKQSVYLMNLRK

SEQ ID NO: 4 - protein, 11 aa, Human immunodeficiency virus
YGRKKRRQRRR

SEQ ID NO: 5 - protein, 12 aa, Artificial Sequence
  Description of Artificial Sequence Synthetic peptide
MYGRKKRRQRRR

SEQ ID NO: 6 - protein, 30 aa, Unknown
  Description of Unknown Galanin sequence
GWTLNSAGYLLGPHAVGNHRSFSDKNGLTS

SEQ ID NO: 7 - protein, 14 aa, Unknown
  Description of Unknown Mastoparan sequence
INLKALAALAKKIL

SEQ ID NO: 8 - protein, 27 aa, Artificial Sequence
  Description of Artificial Sequence Synthetic peptide
GWTLNSAGYLLGKINLKALAALAKKIL

SEQ ID NO: 9 - protein, 16 aa, Drosophila sp.
RQIKIWFQNRRMKWKK

SEQ ID NO: 10 - protein, 9 aa, Artificial Sequence
  Description of Artificial Sequence Synthetic peptide
RRRRRRRRR

SEQ ID NO: 11 - protein, 34 aa, Artificial Sequence
  Description of Artificial Sequence Synthetic polypeptide
DAATATRGRSAASRPTERPRAPARSASRPRRPVE

SEQ ID NO: 12 - protein, 224 aa, Artificial Sequence
  Description of Artificial Sequence Synthetic polypeptide
MYGRKKRRQRRRGGMWTLGRRAVAGLLASPSPAQAQTLTRVPRPAELAPLCGRRGLRTDIDATCTPRRASSNQRGLNQIWNVKKQSVYLMNLRKSGTLGHPGSLDETTYERLAEETLDSLAEFFEDLADKPYTFEDYDVSFGSGVLTVKLGGDLGTYVINKQTPNKQIWLSSPSSGPKRYDWTGKNWVYSHDGVSLHELLAAELTKALKTKLDLSSLAYSGKDA

(19) United States
(12) Patent Application Publication
(10) Pub. No.: US 2020/0377951 A1
(43) Pub. Date: Dec. 3, 2020

(54) FRATAXIN-SENSITIVE MARKERS FOR DETERMINING EFFECTIVENESS OF FRATAXIN REPLACEMENT THERAPY

(71) Applicant: Chondrial Therapeutics, Inc., Bala Cynwyd, PA (US)

(72) Inventor: Joan David Bettoun, Merion Station, PA (US)

(21) Appl. No.: 16/864,125

(22) Filed: Apr. 30, 2020

Related U.S. Application Data
(60) Provisional application No. 62/840,878, filed on Apr. 30, 2019.

(51) Int. Cl. C12Q 1/6883; C07K 14/47; C12N 7/00; C07K 14/005

(52) U.S. Cl. CPC C12Q 1/6883; C07K 14/47; C12N 7/00; C07K 14/005; C12N 2740/16022; C12Q 2600/106; C07K 2319/00; C12N 2740/16071; C12N 2740/16033; C12Q 2600/158

(57) ABSTRACT

The present disclosure is based, at least in part, on providing a set of markers, also referred to herein as FXN-sensitive genomic markers (or FSGMs), the respective expression levels of which are positively or negatively correlated to frataxin (FXN) levels in a cell. Therefore, these FSGMs can be used to determine, evaluate, and/or monitor the effectiveness of FXN replacement therapy in a subject.

### RELATED APPLICATIONS

This application claims priority to U.S. Provisional Application No. 62/840,878, filed on Apr. 30, 2019, the entire contents of which are expressly incorporated herein by reference.

### SEQUENCE LISTING

The instant application contains a Sequence Listing which has been submitted electronically in ASCII format and is hereby incorporated by reference in its entirety. Said ASCII copy, created on Apr. 30, 2020, is named 130197-00302_SL.txt and is 8,523 bytes in size.

### BACKGROUND

Mitochondrial diseases are a group of disorders caused by dysfunctional mitochondria, the cellular organelles that store potential energy in the form of adenosine triphosphate (ATP) molecules and are found in every cell of the human body except mature red blood cells.

Friedreich's Ataxia (FRDA) is the most common inherited ataxia in humans and results from a deficiency of the mitochondrial protein frataxin (FXN), and specifically human frataxin, hFXN). FRDA is a rare disease with an estimated incidence of 1:29,000, a carrier frequency of ˜1:85, and about 4,000-5,000 reported cases in the United States. FRDA is a progressive multisystem disease, typically beginning in mid-childhood. Patients suffer from multiple symptoms, including progressive neurologic and cardiac dysfunction. Other clinical findings can include scoliosis, fatigue, diabetes, visual impairment, and hearing loss. Inheritance is autosomal recessive and is predominantly caused by an inherited GAA triplet expansion in the first intron of both alleles of the hFXN gene. This triplet expansion causes transcriptional repression of the FRDA gene, which results in the production of very small amounts of hFXN in patients. hFXN heterozygotes typically have hFXN levels at ˜50% of normal but are phenotypically normal. hFXN levels of ˜45-70 pg/μl and ˜5-25 pg/μl in whole blood of heterozygotes and patients afflicted with FRDA respectively have been shown to be stable over time (Plasterer et al., 2013).

Currently, there is no FDA-approved treatment for FRDA. Antioxidants and iron chelation have not been overly effective, and, despite treatment, patients typically experience progressive loss of motor control and die, cardiomyopathy being the primary cause of death.

Protein replacement therapy is a well-established approach to metabolic diseases, such as diabetes, lysosomal storage disorders and hemophilia. Work in patient-derived cellular and animal models has demonstrated that replacement of functional FXN can correct or improve the FRDA disease phenotype. However, there is a need in the art for a reliable and efficient assay to measure clinical response and effectiveness of FXN replacement.

### SUMMARY

In one aspect, the present disclosure is based, at least in part, on providing a set of markers, also referred to herein as FXN-sensitive genomic markers (or FSGMs), whose respective levels are positively or negatively correlated to frataxin (FXN) levels in a cell. In some embodiments, the FSGMs of the present disclosure are contrary regulated by FXN gene ablation followed by FXN protein replacement. Thus, said FSGMs of the present disclosure are both associated with FXN deficiency in a subject conversely associated with FXN replacement. The FSGMs disclosed herein were found to be sensitive to FXN and are considered markers of FXN replacement.

Therefore, these FSGMs can be used to determine, evaluate, and/or monitor the effectiveness of FXN replacement therapy in a subject, as described herein. In some embodiments, the effectiveness of FXN replacement therapy in a subject can be determined, evaluated, and/or monitored based on the analysis of one or more FSGM expression profiles before and after administration or initiation of FXN replacement therapy in the subject. Based on the results of the FSGM expression profile analysis, adjustments can be made to the FXN replacement therapy in a subject to, e.g., initiate, increase, decrease or cease FXN replacement therapy in the subject.

Provided in the present disclosure is a method for evaluating FXN replacement therapy by determining an expression profile (a “baseline FXN(−) profile”) for one or more FSGMs in a sample from an FXN deficient patient prior to treatment with an FXN replacement therapy; determining an expression profile (a “FXN replacement profile”) for the one or more FSGMs in a sample from an FXN-deficient patient subsequent to treatment with an FXN replacement therapy; comparing the baseline FXN(−) profile and the FXN replacement profile, and using the comparison to determine effectiveness of the FXN replacement therapy.

In an aspect of the disclosure, determining an FXN expression profile for the FSGMs comprises determining an FXN feature vector of values indicative of expression of the FXN-sensitive genomic markers. FXN feature vectors may reflect the FXN expression profile status of the sample, whether the sample is from an FXN healthy subject, from an FXN deficient patient, or from an FXN deficient patient following FXN replacement therapy.

In another aspect, the present invention provides a method for evaluating effectiveness of frataxin (FXN) replacement therapy, the method comprising (a) determining an FXN replacement expression profile for one or more FSGMs in a sample from an FXN deficient patient following treatment with FXN replacement therapy; (b) comparing the patient FXN replacement expression profile with a baseline FXN(−) expression profile; and (c) using the comparison to determine effectiveness of the FXN replacement therapy; wherein the one or more FSGMs are any one or more markers defined in Table 2, Table 4 and/or FIG. 3.

In one embodiment, the method further comprises determining a baseline FXN(−) expression profile for one or more FXN-sensitive genomic markers (FSGMs) in a sample from a patient exhibiting FXN deficiency prior to FXN replacement therapy.

In one embodiment, the one or more FSGMs comprise at least one or any combination of more than one of a mitochondrial gene, a EGR-family gene, insulin-like gene, ribosome depletion response gene, mitochondrial energy production gene, proteasome regulation gene, ribosomal function gene, respiratory chain gene, cardiac muscle development gene, macromolecule catabolism gene, a translational initiation gene, mitochondrial components gene, oxidative phosphorylation gene, negative regulation of macromolecule metabolic process gene, or regulation of apoptotic process gene.

In another embodiment, the one or more FSGMs comprise a gene encoding a secreted protein or a secreted protein, e.g., a secreted protein defined in Table 2. In one embodiment, the one or more FSGMs comprise one or more of CYR61, ADAMTS1, ASPN, FAM177A, IGF1, LOX, NRTN, SERPINE1, STC1, and THBS1. In another embodiment, the one or more FSGMs comprise CYR61.

In another embodiment, the one or more FSGMs comprise one or more of NR4A1, PTP4A1, ATF3, BTG2, EGR1, EGR2, EGR3, CYR61, and ABCE1.

In another embodiment, the one or more FSGMs comprise one or more of EGR1, EGR2, EGR3 and IGF1.

In another embodiment, the one or more FSGMs comprise one or more of MT-ND1, MT-ND2, MT-ND3, MT-ND4, MT-CO3, MT-ATP6, MT-ATP8, and CYCS.

In another embodiment, the one or more FSGMs comprise one or more of OPS2, VBP1, PSMA3, SLIRP, CUL2, DCUN1D1, UBE2D3, ZNRF1, RNF2, and LAMP2.

In another embodiment, the one or more FSGMs comprise one or more of RPS15A, EIF1AX, RPL24, RPL32, RPL26, RPL10, RPL39, RPL38, RPS27L, and ABCE1.

In another embodiment, the one or more FSGMs comprise one or more of MT-ND1, MT-ND2, MT-ND3, MT-ND4, MT-CO3, and CYCS.

In another embodiment, the one or more FSGMs comprise one or more of NR4A1, EGR1, EGR3, ADAMTS1, THBS1, SERPINE1, IGF1, PTGS2, and CYR61.

In another embodiment, the one or more FSGMs comprise one or more of PSMA3, CUL2, UBE2D3, ZNRF1, RPS15A, RPL24, RPL32, RPL26, RPL10, RPL39, and RPL38.

In another embodiment, the one or more FSGMs comprise one or more of ABCE1, RPS15A, EIF1AX, RPL24, RPL32, RPL26, RPL10, RPL39, and RPL38.

In another embodiment, the one or more FSGMs comprise one or more of MT-ND1, MT-ND2, MT-ND3, MT-ND4, MT-CO3, MT-ATP6, MT-ATP8, CYCS, TMEM-126A, MAOA, and ABCE1.

In another embodiment, the one or more FSGMs comprise one or more of MT-ND1, MT-ND2, MT-ND3, MT-ND4, MT-CO3, MT-ATP6, and MT-ATP8.

In another embodiment, the one or more FSGMs comprise one or more of ABCE1, RPL26, RPL38, RPL10, RPL32, RPS15A, RPL24, RPL39, SLIRP, COPS2, DCUN1D1, RNF2, EGR1, BTG2, ATF3, PTGS2, IGF1, SERPINE1, and THBS1.

In another embodiment, the one or more FSGMs comprise one or more of RPL26, THBS1, SERPINE1, IGF1, PTGS2, RPL10, RPS27L, CYCS, ATF3, BTG2, EGR1, EGR3, and CYR61.

In one embodiment, the one or more FSGMs are upregulated following treatment with FXN replacement therapy.

In one embodiment, the one or more FSGMs that are upregulated following treatment with FXN replacement therapy are mt-RNR1, mt-RNR2, ADNP, AI480526, C230034O21RIK, CCDCl85B, CCDCl85C, CTCFL, NRTN, PDE4A, PHF1, RPL37RT, SLC26A10, SNORD17, SUV420H2, WNK2, YAM1 or ZNRF1.

In one embodiment, the one or more FSGMs are downregulated following treatment with FXN replacement therapy.

In one embodiment, the one or more FSGMs that are downregulated following treatment with FXN replacement therapy are CYR61, mt-ATP6, mt-ATP8, mt-CO2, mt-CO3, mt-ND1, mt-ND2, mt-ND3 and mt-ND4, EGR1, EGR2, EGR3, IGF1, LAMP2, or SLIRP.

In another embodiment, determining an FXN expression profile for FSGMs comprises determining an FXN feature vector of values indicative of expression of the FSGMs. In one embodiment, the method comprises using the comparison to determine effectiveness of the FXN replacement therapy comprising determining first and second FXN feature vectors for the patient FXN replacement expression profile and the baseline FXN(−) expression profile respectively and determining a distance between the feature vectors.

In one embodiment, determining the distance between the feature vectors comprises determining a scalar product of the first and second feature vectors.

In one embodiment, the method further comprises determining a third feature vector for a normal FXN expression profile for the FSGMs for a healthy subject.

In one embodiment, the method further comprises determining a distance between the second and third feature vectors.

In one embodiment, the method further comprises determining a distance between the first and third feature vectors, and normalizing the distance between the first and third feature vectors to the distance between the second and third feature vectors.

In one embodiment, the method further comprises using the normalized distance to determine effectiveness of the FXN replacement therapy.

In one embodiment, the expression profile is determined by any one of sequencing, hybridization or amplification of the sample RNA.

In one embodiment, the expression profile is determined by HPLC/UV-Vis spectroscopy, enzymatic analysis, mass spectrometry, NMR, immunoassay, ELISA, or any combination thereof.

In another embodiment, the methods of the invention further comprise modifying treatment with the FXN replacement therapy when the FXN replacement therapy is indicated as being ineffective.

In one embodiment, the patient is suffering from Freidrich's Ataxia (FRDA).

In one embodiment, the method further comprises obtaining a biological sample from a patient exhibiting FXN deficiency.

In one aspect, the present invention provides a composition for determining the expression profile of FSGMs, the composition comprising reagents for the detection of at least one or more FSGMs described in Table 2, Table 4 and/or FIG. 3.

In another aspect, the present invention provides a method for treatment of a mitochondrial disease, the method comprising providing a sample from a subject suffering from FXN deficiency, determining an FXN expression profile in the sample for one or more FXN-sensitive genomic markers (FSGMs), comparing the FXN expression profile of the sample with at least one other expression profile selected from the group consisting of normal FXN expression profile for one or more FSGMs, baseline FXN(−) expression profile for one or more FSGMs, and an FXN replacement expression profile for one or more FSGMs, classifying the sample FXN expression profile as corresponding to a normal FXN expression profile, baseline FXN(−) expression profile or an FXN replacement expression profile, and initiating, increasing or decreasing the dosage of FXN replacement therapy to be administered to the subject based on the classification of the sample FXN expression profile.

In another aspect, the present invention provides a method for treatment of a mitochondrial disease, the method comprising determining expression of one or more FXN-sensitive genomic markers (FSGMs) in a sample from a suffering from FXN deficiency, wherein the one or more FSGMs are any one or more markers defined in Table 2, Table 4 and/or FIG. 3, and initiating, increasing or decreasing the dosage of an FXN replacement therapy to be administered to the subject based on the expression of the one or more FSGMs.

In one embodiment, the method comprises providing or obtaining a sample from a subject suffering from FXN deficiency.

In one embodiment, the mitochondrial disease is Friedrich's Ataxia (FRDA).

In another embodiment, the one or more FSGMs comprise a secreted protein, e.g., a secreted protein defined in Table 2. In one embodiment, the one or more FSGMs comprise one or more of CYR61, ADAMTS1, ASPN, FAM177A, IGF1, LOX, NRTN, SERPINE1, STC1, and THBS1. In another embodiment, the one or more FSGMs comprise CYR61.

In one embodiment, the one or more FSGMs comprise one or more of NR4A1, PTP4A1, ATF3, BTG2, EGR1, EGR2, EGR3, CYR61, and ABCE1.

In another embodiment, the one or more FSGMs comprise one or more of EGR1, EGR2, EGR3 and IGF1.

In another embodiment, the one or more FSGMs comprise one or more of MT-ND1, MT-ND2, MT-ND3, MT-ND4, MT-CO3, MT-ATP6, MT-ATP8, and CYCS.

In another embodiment, the one or more FSGMs comprise one or more of OPS2, VBP1, PSMA3, SLIRP, CUL2, DCUN1D1, UBE2D3, ZNRF1, RNF2, and LAMP2.

In another embodiment, the one or more FSGMs comprise one or more of RPS15A, EIF1AX, RPL24, RPL32, RPL26, RPL10, RPL39, RPL38, RPS27L, and ABCE1.

In another embodiment, the one or more FSGMs comprise one or more of MT-ND1, MT-ND2, MT-ND3, MT-ND4, MT-CO3, and CYCS.

In another embodiment, the one or more FSGMs comprise one or more of NR4A1, EGR1, EGR3, ADAMTS1, THBS1, SERPINE1, IGF1, PTGS2, and CYR61.

In another embodiment, the one or more FSGMs comprise one or more of PSMA3, CUL2, UBE2D3, ZNRF1, RPS15A, RPL24, RPL32, RPL26, RPL10, RPL39, and RPL38.

In another embodiment, the one or more FSGMs comprise one or more of ABCE1, RPS15A, EIF1AX, RPL24, RPL32, RPL26, RPL10, RPL39, and RPL38.

In another embodiment, the one or more FSGMs comprise one or more of MT-ND1, MT-ND2, MT-ND3, MT-ND4, MT-CO3, MT-ATP6, MT-ATP8, CYCS, TMEM-126A, MAOA, and ABCE1.

In another embodiment, the one or more FSGMs comprise one or more of MT-ND1, MT-ND2, MT-ND3, MT-ND4, MT-CO3, MT-ATP6, and MT-ATP8.

In another embodiment, the one or more FSGMs comprise one or more of ABCE1, RPL26, RPL38, RPL10, RPL32, RPS15A, RPL24, RPL39, SLIRP, COPS2, DCUN1D1, RNF2, EGR1, BTG2, ATF3, PTGS2, IGF1, SERPINE1, and THBS1.

In another embodiment, the one or more FSGMs comprise one or more of RPL26, THBS1, SERPINE1, IGF1, PTGS2, RPL10, RPS27L, CYCS, ATF3, BTG2, EGR1, EGR3, and CYR61.

In another aspect, the present invention provides a kit for detecting one or more frataxin-sensitive genomic marker (FSGM) in a biological sample from a subject exhibiting frataxin (FXN) deficiency or being treated for FXN deficiency, comprising one or more reagents for measuring the level of the one or more FSGM in the biological sample from the subject, wherein the one or more FSGM comprises one or more FSGMs selected from Table 2, Table 4 and/or FIG. 3, and a set of instructions for measuring the level of the FSGM.

In one embodiment, the reagent is an antibody that binds to the one or more frataxin-sensitive genomic marker (FSGM) or an oligonucleotide that is complementary to the corresponding mRNA of the one or more FSGM.

In one embodiment, the one or more FSGMs comprise a secreted protein, e.g., a secreted protein defined in Table 2. In one embodiment, the one or more FSGMs comprise one or more of CYR61, ADAMTS1, ASPN, FAM177A, IGF1, LOX, NRTN, SERPINE1, STC1, and THBS1. In another embodiment, the one or more FSGMs comprise CYR61.

In another aspect, the present invention provides a panel for use in a method of monitoring or evaluating the efficacy of frataxin (FXN) replacement therapy, the panel comprising one or more detection reagents, wherein each detection reagent is specific for the detection of one or more frataxin-sensitive genomic marker (FSGM), wherein the one or more FSGM comprises one or more markers selected from Table 2, Table 4 and/or FIG. 3.

In one embodiment, the frataxin-sensitive genomic marker (FSGM) comprises at least two or more FSGMs.

In one embodiment, the one or more FSGMs comprise a secreted protein, e.g., a secreted protein defined in Table 2. In one embodiment, the one or more FSGMs comprise one or more of CYR61, ADAMTS1, ASPN, FAM177A, IGF1, LOX, NRTN, SERPINE1, STC1, and THBS1. In another embodiment, the one or more FSGMs comprise CYR61.

In another aspect, the present invention provides a kit comprising a panel of the invention and a set of instructions for obtaining information relating to frataxin (FXN) replacement therapy based on a level of the one or more frataxin-sensitive genomic markers (FSGMs).

In another aspect, the present invention provides a method of detecting one or more frataxin-sensitive genomic markers (FSGMs) in a biological sample from a patient suffering from a frataxin (FXN) deficiency, optionally wherein the patient is being treated with a FXN replacement therapy, by contacting the biological sample, or a portion thereof, with one or more detection reagents specific for detection of one or more FSGMs, wherein the one or more FSGMs comprises one or more FSGMs selected from Table 2, Table 4 and/or FIG. 3. In one embodiment, the sample is contacted with one or more detection reagents specific for detection of one or more FSGMs. In another embodiment, a portion of the sample, such as an isolated or purified nucleic acid, or protein, can be contacted with one or more detection reagents specific for detection of one or more FSGMs.

In one embodiment, the one or more FSGMs comprise a secreted protein, e.g., a secreted protein defined in Table 2. In one embodiment, the one or more FSGMs comprise one or more of CYR61, ADAMTS1, ASPN, FAM177A, IGF1, LOX, NRTN, SERPINE1, STC1, and THBS1. In another embodiment, the one or more FSGMs comprise CYR61.

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Unless otherwise indicated, the word “or” in the description and claims is considered to be the inclusive “or” (having the meaning of and/or) rather than the exclusive or, and indicates at least one of, or any combination of items it conjoins.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the disclosure are described below with reference to figures attached hereto that are listed following this paragraph. Identical features that appear in more than one figure are generally labeled with a same label in all the figures in which they appear.

FIG. 1 shows clusters generated by string analysis of predicted interactions of protein products of 85 FXN-sensitive genomic markers (FSGMs) from Table 2, in accordance with an embodiment of the disclosure.

FIG. 2 is a photo of a representative Western blot showing FXN levels in normal dermal fibroblasts (Norm #23971) and FDRA patient-derived fibroblasts (FA_#03816 and FA_#68).

FIG. 3 is a graph showing a baseline FXN (−) expression profile in FDRA-derived fibroblasts FA-GM03816, FA-GM04078, FA-4654 and FA-4675 treated with vehicle and compared to normal fibroblast control N-GM07522 and N-GM23971, in accordance with an embodiment of the disclosure.

FIG. 4A is a graph showing gene expression analysis in FRDA patient-derived fibroblasts, illustrating that EGR1, EGR2, EGR3, and IGF1 are overall upregulated in the FRDA patient-derived fibroblasts compared to normal fibroblasts. FIG. 4B is a graph showing the effect of the FXN fusion protein described in Example 1 on the expression of hFXN, EGR1, EGR2, EGR3, and IGF1 in FDRA-derived fibroblasts FA-68, compared to vehicle-treated cells, in accordance with an embodiment of the disclosure.

FIG. 5 is a schematic of the procedure for evaluating the FXN-induced signature, in accordance with an embodiment of the disclosure.

FIG. 6 is a photo of the Western blot showing the amount of FXN protein in FXN knockdown (KD) HK293 clones A2 and A6 and in the scrambled control clone. FIG. 6 also shows a table showing the results of quantification of the amount of the FXN protein in the Western blot.

FIG. 7 is a bar graph showing the amount of CYR61 protein in the media from FXN-KD and scrambled control HEK293 cells treated with vehicle (black bars) or FXN fusion protein (gray bars).

FIG. 8 is a bar graph showing the amount of CYR61 protein in the media from the scrambled control cells transfected with an empty vector (KD-SRBL+V); scrambled control cells transfected with hFXN (SRBL 5+hFXN); hFXN-KD cells transfected with an empty vector (KD-FXN+V); and hFXN-KD cells transfected with hFXN (KD-FXN+hFXN).

FIG. 9 is a bar graph showing the amount of FXN protein per total cellular protein in the WT mouse ES clone and the homozygous mouse ES clone B9-46 which has been treated with control or an agent to induce the FXN knockout (knockout agent).

FIG. 10A is a bar graph showing the amount of CYR61 expressed in mouse ES B9 cells treated with a control agent or an agent to induce knockdown of the FXN gene. FIG. 10B is a bar graph showing the amount of secreted CYR61 protein in the media from mouse ES B9 cells treated with a control agent or an agent to induce knockdown of the FXN gene.

### DETAILED DESCRIPTION OF THE INVENTION

### A. Overview

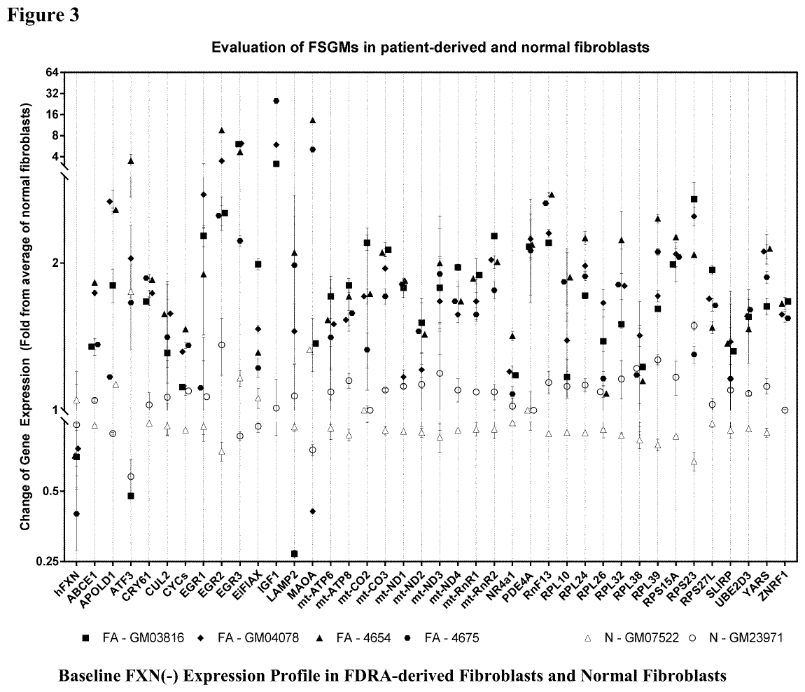

In one aspect, the present disclosure is based, at least in part, on providing a set of markers, also referred to herein as FXN-sensitive genomic markers (or FSGMs), whose respective levels are positively or negatively correlated to frataxin (FXN) levels in a cell. In some embodiments, the FSGMs of the present disclosure are contrary regulated by FXN gene ablation followed by FXN protein replacement. Thus, said FSGMs of the present disclosure are both associated with FXN deficiency in a subject and conversely associated with FXN replacement. The FSGMs disclosed herein were found to be sensitive to FXN and are considered markers of FXN replacement. Therefore, these FSGMs can be used to determine and/or monitor the effectiveness of FXN replacement therapy in a subject, as described herein. In one embodiment, the FSGMs comprise one or more markers selected from Table 2, Table 4 and/or FIG. 3. In one embodiment, the FSGMs comprise a secreted protein, e.g., a secreted protein defined in Table 2. In one embodiment, the FSGMs comprise one or more of CYR61, ADAMTS1, ASPN, FAM177A, IGF1, LOX, NRTN, SERPINE1, STC1, and THBS1. In another embodiment, the FSGMs comprise CYR61.

In some embodiments, the effectiveness of FXN replacement therapy in a subject can be determined, evaluated, and/or monitored based on the analysis of one or more FSGM expression profiles before and after administration or initiation of FXN replacement therapy in the subject. Based on the results of the FSGM expression profile analysis, adjustments can be made to the FXN replacement therapy in a subject to, e.g., initiate, increase, decrease or cease FXN replacement therapy in the subject.

### B. Definitions

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention.

As used herein, the singular forms “a”, “and”, and “the” include plural references unless the context clearly dictates otherwise. All technical and scientific terms used herein have the same meaning.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “amplification” refers to any known in vitro procedure for obtaining multiple copies (“amplicons”) of a target nucleic acid sequence or its complement or fragments thereof. In vitro amplification refers to production of an amplified nucleic acid that may contain less than the complete target region sequence or its complement. Known in vitro amplification methods include, e.g., transcription-mediated amplification, replicase-mediated amplification, polymerase chain reaction (PCR) amplification, ligase chain reaction (LCR) amplification and strand-displacement amplification (SDA including multiple strand-displacement amplification method (MSDA)). Replicase-mediated amplification uses self-replicating RNA molecules, and a replicase such as Q-β-replicase (e.g., Kramer et al., U.S. Pat. No. 4,786,600). PCR amplification is well known and uses DNA polymerase, primers and thermal cycling to synthesize multiple copies of the two complementary strands of DNA or cDNA (e.g., Mullis et al., U.S. Pat. Nos. 4,683,195, 4,683,202, and 4,800,159). LCR amplification uses at least four separate oligonucleotides to amplify a target and its complementary strand by using multiple cycles of hybridization, ligation, and denaturation (e.g., EP Pat. App. Pub. No. 0 320 308). SDA is a method in which a primer contains a recognition site for a restriction endonuclease that permits the endonuclease to nick one strand of a hemimodified DNA duplex that includes the target sequence, followed by amplification in a series of primer extension and strand displacement steps (e.g., Walker et al., U.S. Pat. No. 5,422,252). Two other known strand-displacement amplification methods do not require endonuclease nicking (Dattagupta et al., U.S. Pat. Nos. 6,087,133 and 6,124,120 (MSDA)). Those skilled in the art will understand that the oligonucleotide primer sequences of the present invention may be readily used in any in vitro amplification method based on primer extension by a polymerase. (see generally Kwoh et al., 1990, Am. Biotechnol. Lab. 8:14-25 and (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 2000, Molecular Cloning—A Laboratory Manual, Third Edition, CSH Laboratories). As commonly known in the art, the oligos are designed to bind to a complementary sequence under selected conditions.

As used herein, the term “marker” or “biomarker” is a biological molecule, or a panel of biological molecules, whose expression level is correlated, e.g., either positively or negatively, with FXN levels.

As used herein, a marker or biomarker of the invention whose respective levels are positively or negatively correlated to frataxin (FXN) levels in a cell is referred to as a “Frataxin-sensitive genomic marker” or “FSGM”. In some embodiments, the FSGMs of the present disclosure are contrary regulated by FXN gene ablation followed by FXN protein replacement. Thus, in some embodiments, the FSGMs of the present disclosure are both associated with FXN deficiency in a subject and conversely associated with FXN replacement. An FSGM of the invention can be used to detect and/or monitor FXN levels in a sample, e.g., a cell or tissue sample. In preferred embodiments, an FSGM is selected from those listed in Table 2, Table 4 or FIG. 3, human genes and proteins in Table 2, and human homologues of genes and proteins in Table 2. Reference to FSGMs in Table 2, Table 4 and FIG. 3, as used herein, is understood to include reference to any mutants, variants, derivatives, or orthologs thereof.

The term “control sample” or “control,” as used herein, refers to any clinically relevant comparative sample, including, for example, a sample from an FXN healthy subject (i.e., a subject with a normal FXN level), a normal FXN expression profile, a sample from an FXN deficient subject (i.e., a subject completely or partially lacking FXN expression), a baseline FXN(−) expression profile, or a sample from a subject following FXN replacement therapy, or an FXN replacement expression profile. A control sample can also be a sample from a subject from an earlier time point, e.g., prior to treatment with FXN replacement therapy. A control sample can be a purified sample, protein, and/or nucleic acid provided with a kit. Such control samples can be diluted, for example, in a dilution series to allow for quantitative measurement of levels of analytes, e.g., markers, in test samples. A control sample may include a sample derived from one or more subjects. A control sample may also be a sample made at an earlier time point from the subject to be assessed. For example, the control sample could be a sample taken from the subject to be assessed before treatment with FXN replacement therapy. The control sample may also be a sample from an animal model, or from a tissue or cell line derived from the animal model of a mitochondrial disease such as FRDA. The level of activity or expression of one or more FSGMs (e.g., 1, 2, 3, 4, 5, 6, 7, 8, or 9 or more FSGMs) in a control sample consists of a group of measurements that may be determined, e.g., based on any appropriate statistical measurement, such as, for example, measures of central tendency including average, median, or modal values. In one embodiment, “different from a control” is preferably statistically significantly different from a control.

As used herein, “changed, altered, increased or decreased” is understood as having a level of the one or more FSGM to be detected at a level that is statistically different, e.g., increased or decreased, as compared to a control sample or threshold value, e.g., from an FXN healthy subject (i.e., a subject with a normal FXN level), or a sample from an FXN deficient subject (i.e., a subject lacking FXN expression). Changed, altered, increased or decreased, as compared to control or threshold value, can also include a difference in the rate of change of the level of one or more FSGMs obtained in a series of at least two subject samples obtained over time. Determination of statistical significance is within the ability of those skilled in the art and can include any acceptable means for determining and/or measuring statistical significance, such as, for example, the number of standard deviations from the mean that constitute a positive or negative result, an increase in the detected level of an FSGM in a sample versus a control, wherein the increase is above some threshold value, or a decrease in the detected level of an FSGM in a sample versus a control, wherein the decrease is below some threshold value.

As used herein, “detecting”, “detection”, “determining”, and the like are understood to refer to identification of the presence and/or level of one or more FSGMs selected from Table 2, Table 4 and/or FIG. 3.

As used herein, the term “DNA” or “RNA” molecule or sequence (as well as sometimes the term “oligonucleotide”) refers to a molecule comprised generally of the deoxyribonucleotides adenine (A), guanine (G), thymine (T) and/or cytosine (C). In “RNA”, T is replaced by uracil (U).

As used herein, the terms “FXN deficient patient” and “FXN deficient subject” refer to a subject that has a reduced level of FXN expression or activity as compared to a normal control subject. Certain diseases result in FXN deficiencies in patients, including mitochondrial diseases such as Friedreich's Ataxia (FRDA).

As used herein, the term “FXN replacement therapy” refers to replacement of frataxin in a subject which results in increased expression or activity of frataxin in the subject. The FXN replacement therapy may be carried out by FXN protein delivery or through delivery of a nucleic acid encoding FXN to a subject. FXN protein delivery to the subject can include delivery of FXN protein or delivery of a FXN fusion protein. As used herein, the term “FXN fusion protein” refers to full length FXN or a fragment of FXN fused to a full length or a fragment of a different protein, or to a peptide. In some embodiments, an FXN fusion protein comprises full-length hFXN (SEQ ID NO: 1) or mature hFXN (SEQ ID NO: 2), as described herein. In some embodiments, the FXN protein or fragment thereof is fused to a cell penetrating peptide (CPP). In some embodiments, the CPP is an HIV-TAT polypeptide.

As used herein, the terms “disorders”, “diseases”, and “abnormal state” are used inclusively and refer to any deviation from the normal structure or function of any part, organ, or system of the body (or any combination thereof). A specific disease is manifested by characteristic symptoms and signs, including biological, chemical, and physical changes, and is often associated with a variety of other factors including, but not limited to, demographic, environmental, employment, genetic, and medically historical factors. An early stage disease state includes a state wherein one or more physical symptoms are not yet detectable. Certain characteristic signs, symptoms, and related factors can be quantitated through a variety of methods to yield important diagnostic information.

As used herein, the term “mitochondrial disease” refers to a disease which is the result of either inherited or spontaneous mutations in mtDNA or nDNA which leads to altered functions of the proteins or RNA molecules that normally reside in mitochondria, which decreases the functions of the mitochondria to induce diseases of various types in, for example, the central nervous system, skeletal muscles, heart, eyes, liver, kidneys, large intestine (colon), small intestine, internal ear and pancreas; as well as blood, skin and endocrine glands. In one non-limiting embodiment, the mitochondrial disease is Friedrich's Ataxis (FRDA).

As used herein, a sample obtained at an “earlier time point” is a sample that was obtained at a sufficient time in the past such that clinically relevant information could be obtained in the sample from the earlier time point as compared to the later time point. In certain embodiments, an earlier time point is at least 1, 2, 3, 4, 5, 6, 7, 8, 9, 10, 11, 12, 13, 14, 15, 16, 17, 18, 19, 20, 21, 22, or 23 hours, or 1, 2, 3, 4, 5, 6, or 7 days earlier. In some embodiments, an earlier time point is at least one, two, three or four weeks earlier. In certain embodiments, an earlier time point is at least six weeks earlier. In certain embodiments, an earlier time point is at least two months earlier. In certain embodiments, an earlier time point is at least three months earlier. In certain embodiments, an earlier time point is at least six months earlier. In certain embodiments, an earlier time point is at least nine months earlier. In certain embodiments, an earlier time point is at least one year earlier. Multiple subject samples (e.g., 3, 4, 5, 6, 7, or more) can be obtained at regular or irregular intervals over time and analyzed for trends in changes in FSGM levels. Appropriate intervals for testing for a particular subject can be determined by one of skill in the art based on ordinary considerations.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, or protein, or both.

The term “expression profile” is used to include a genomic expression profile, meaning an expression profile of RNAs, or specifically of mRNAs or transcripts, or a protein expression profile. As used herein, expression profile may refer to a set of data obtained for mRNA expression. It may refer to the raw data in the readings of a PCR apparatus for example, or to the normalized expression values. Expression profiles may be determined by any convenient means for measuring a level of a nucleic acid sequence such as quantitative hybridization of mRNA, labeled mRNA, amplified mRNA, cDNA, etc., quantitative PCR, and other techniques known to a person skilled in the art or described herein. Expression profiles enable analysis of differential gene expression between two or more samples, between samples and control, as well as between samples and thresholds. An expression profile can also be determined by any means known to a person skilled in the art or described herein for measuring the level of a protein or a polypeptide, e.g., mass spectrometry, immunodetection assays, e.g., ELISA, etc.

As referred to herein, the term “FXN expression profile” includes any one of the following three FXN expression profiles: a normal FXN expression profile, a baseline FXN(−) expression profile, or an FXN replacement expression profile. As used herein, the baseline FXN(−) expression profile can also be referred to as the “threshold level” of expression of an FSGM. The baseline FXN(−) expression profile can also be used as a control.

As referred to herein, the term “normal FXN profile” refers to the expression profile of one or more FSGMs in a sample from a normal patient (i.e., a patient that is not FXN deficient).

As referred to herein, the term “baseline FXN(−) profile” refers to the expression profile of one or more FSGMs in a sample from an FXN deficient patient prior to treatment with an FXN replacement therapy.

As referred to herein, the term “FXN replacement profile” refers to the expression profile for one or more FSGMs in a sample from an FXN-deficient patient subsequent to treatment with an FXN replacement therapy.

A “higher level of expression”, “higher level”, “increased level,” and the like of an FSGM refers to an expression level in a test sample that is greater than the standard error of the assay employed to assess expression, and is preferably at least 25% more, at least 50% more, at least 75% more, at least two, at least three, at least four, at least five, at least six, at least seven, at least eight, at least nine, or at least ten times the expression level of the FSGM in a control sample (e.g., a sample from a healthy subject, a sample from an FXN deficient subject, or a sample from a subject following FXN replacement therapy) and preferably, the average expression level of the FSGM or FSGMs in several control samples.

As used herein, the term “hybridization,” as in “nucleic acid hybridization,” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 2000, supra and Ausubel et al., 1994, supra, or further in Higgins and Hames (Eds.) “Nucleic acid hybridization, a practical approach” IRL Press Oxford, Washington D.C., (1985)) and are commonly known in the art. In the case of a hybridization to a nitrocellulose filter (or other such support like nylon), as for example in the well-known Southern blotting procedure, a nitrocellulose filter can be incubated overnight at a temperature representative of the desired stringency condition (60-65° C. for high stringency, 50-60° C. for moderate stringency and 40-45° C. for low stringency conditions) with a labeled probe in a solution containing high salt (6×SSC or 5×SSPE), 5×Denhardt's solution, 0.5% SDS, and 100 μg/ml denatured carrier DNA (e.g., salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The salt and SDS concentration of the washing solutions may also be adjusted to accommodate for the desired stringency. The selected temperature and salt concentration is based on the melting temperature (Tm) of the DNA hybrid. Of course, RNA-DNA hybrids can also be formed and detected. In such cases, the conditions of hybridization and washing can be adapted according to well-known methods by the person of ordinary skill. Stringent conditions will be preferably used (Sambrook et al., 2000, supra). Other protocols or commercially available hybridization kits (e.g., ExpressHyb® from BD Biosciences Clonetech) using different annealing and washing solutions can also be used as well known in the art. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed).

As used herein, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art. Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul Nucl. Acids Res. 25 (1977), 3389-3402). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff Proc. Natl. Acad. Sci., USA, 89, (1989), 10915) uses alignments (B) of 50, expectation (E) of 10, M=5, N=4, and a comparison of both strands. Moreover, the present invention also relates to nucleic acid molecules the sequence of which is degenerate in comparison with the sequence of an above-described hybridizing molecule. When used in accordance with the present invention the term “being degenerate as a result of the genetic code” means that due to the redundancy of the genetic code different nucleotide sequences code for the same amino acid. The present invention also relates to nucleic acid molecules which comprise one or more mutations or deletions, and to nucleic acid molecules which hybridize to one of the herein described nucleic acid molecules, which show (a) mutation(s) or (a) deletion(s).

The term “including” is used herein to mean, and is used interchangeably with, the phrase “including but not limited to.”

As used herein, a “label” refers to a molecular moiety or compound that can be detected or can lead to a detectable signal. A label is joined, directly or indirectly, to a molecule, such as an antibody, a nucleic acid probe or the protein/antigen or nucleic acid to be detected (e.g., an amplified sequence). Direct labeling can occur through bonds or interactions that link the label to the nucleic acid (e.g., covalent bonds or non-covalent interactions), whereas indirect labeling can occur through the use of a “linker” or bridging moiety, such as oligonucleotide(s) or small molecule carbon chains, which is either directly or indirectly labeled. Bridging moieties may amplify a detectable signal. Labels can include any detectable moiety (e.g., a radionuclide, ligand such as biotin or avidin, enzyme or enzyme substrate, reactive group, chromophore such as a dye or colored particle, luminescent compound including a bioluminescent, phosphorescent or chemiluminescent compound, and fluorescent compound). Preferably, the label on a labeled probe is detectable in a homogeneous assay system, i.e., in a mixture, the bound label exhibits a detectable change compared to an unbound label.

The terms “level of expression of a gene”, “gene expression level”, “level of an FSGM”, and the like refer to the level of mRNA, as well as pre-mRNA nascent transcript(s), transcript processing intermediates, mature mRNA(s) and degradation products, or the level of protein, encoded by the gene in the cell. The “level” of one of more FSGMs means the absolute or relative amount or concentration of the FSGM in the sample.

A “lower level of expression” or “lower level” or “decreased level” and the like of an FSGM refers to an expression level in a test sample that is less than 90%, 85%, 80%, 75%, 70%, 65%, 60%, 55%, 50%, 45%, 40%, 35%, 30%, 25%, 20%, 15%, or 10% of the expression level of the FSGM in a control sample (e.g., a sample from a healthy subject, a sample from an FXN deficient subject, or a sample from a subject following FXN replacement therapy) and preferably, the average expression level of the FSGM in several control samples.

As used herein, “nucleic acid molecule” or “polynucleotides”, refers to a polymer of nucleotides, including an FSGM. Non-limiting examples thereof include DNA (e.g., genomic DNA, cDNA), RNA molecules (e.g., mRNA) and chimeras thereof. The nucleic acid molecule can be obtained by cloning techniques or synthesized. DNA can be double-stranded or single-stranded (coding strand or non-coding strand [antisense]). Conventional ribonucleic acid (RNA) and deoxyribonucleic acid (DNA) are included in the term “nucleic acid” and polynucleotides as are analogs thereof. A nucleic acid backbone may comprise a variety of linkages known in the art, including one or more of sugar-phosphodiester linkages, peptide-nucleic acid bonds (referred to as “peptide nucleic acids” (PNA); Hydig-Hielsen et al., PCT Intl Pub. No. WO 95/32305), phosphorothioate linkages, methylphosphonate linkages or combinations thereof. Sugar moieties of the nucleic acid may be ribose or deoxyribose, or similar compounds having known substitutions, e.g., 2′ methoxy substitutions (containing a 2′-O-methylribofuranosyl moiety; see PCT No. WO 98/02582) and/or 2′ halide substitutions. Nitrogenous bases may be conventional bases (A, G, C, T, U), known analogs thereof (e.g., inosine or others; see The Biochemistry of the Nucleic Acids 5-36, Adams et al., ed., 11th ed., 1992), or known derivatives of purine or pyrimidine bases (see, Cook, PCT Int'l Pub. No. WO 93/13121) or “abasic” residues in which the backbone includes no nitrogenous base for one or more residues (Arnold et al., U.S. Pat. No. 5,585,481). A nucleic acid may comprise only conventional sugars, bases and linkages, as found in RNA and DNA, or may include both conventional components and substitutions (e.g., conventional bases linked via a methoxy backbone, or a nucleic acid including conventional bases and one or more base analogs). An “isolated nucleic acid molecule”, as is generally understood and used herein, refers to a polymer of nucleotides, and includes, but should not limited to DNA and RNA. The “isolated” nucleic acid molecule is purified from its natural in vivo state, obtained by cloning or chemically synthesized.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well-known methods. While they are usually in a single-stranded form, they can be in a double-stranded form and even contain a “regulatory region”. They can contain natural rare or synthetic nucleotides. They can be designed to enhance a chosen criterion like stability for example. Chimeras of deoxyribonucleotides and ribonucleotides may also be within the scope of the present invention.

As used herein, “one or more” is understood as encompassing each value 1, 2, 3, 4, 5, 6, 7, 8, 9, 10, and any value greater than 10.

The term “or” is used inclusively herein to mean, and is used interchangeably with, the term “and/or,” unless context clearly indicates otherwise.

As used herein, “patient” or “subject” can mean either a human or non-human animal, preferably a mammal. By “subject” is meant any animal, including horses, dogs, cats, pigs, goats, rabbits, hamsters, monkeys, guinea pigs, rats, mice, lizards, snakes, sheep, cattle, fish, and birds. A human subject may be referred to as a patient.

As used herein, a “probe” is meant to include a nucleic acid oligomer or oligonucleotide that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled. A probe can be produced by molecular cloning of a specific DNA sequence or it can also be synthesized. Numerous primers and probes which can be designed and used in the context of the present invention can be readily determined by a person of ordinary skill in the art to which the present invention pertains.

As used herein, a “reference level” of an FSGM may be an absolute or relative amount or concentration of the FSGM, a presence or absence of the FSGM, a range of amount or concentration of the FSGM, a minimum and/or maximum amount or concentration of the FSGM, a mean amount or concentration of the FSGM, and/or a median amount or concentration of the FSGM; and, in addition, “reference levels” of combinations of FSGMs may also be ratios of absolute or relative amounts or concentrations of two or more FSGMs with respect to each other. Appropriate positive and negative reference levels of FSGMs for a particular disease state, phenotype, or lack thereof may be determined by measuring levels of desired FSGMs in one or more appropriate subjects, and such reference levels may be tailored to specific populations of subjects (e.g., a reference level may be age-matched so that comparisons may be made between FSGM levels in samples from subjects of a certain age and reference levels for a particular disease state, phenotype, or lack thereof in a certain age group). Such reference levels may also be tailored to specific techniques that are used to measure levels of FSGMs in biological samples (e.g., LC-MS, GC-MS, etc.), where the levels of FSGMs may differ based on the specific technique that is used.

As used herein, “sample” or “biological sample” includes a specimen or culture obtained from any source. In some embodiments, a sample includes any specimen or culture that comprises cells in which FXN expression profile may be analyzed. In some embodiments, a sample includes any specimen or culture from a subject deficient in FXN or a subject being treated with FXN replacement therapy. For example, biological samples can be obtained from a body fluid sample such as blood (including any blood product, such as whole blood, plasma, serum, or specific types of cells of the blood), urine, saliva, or seminal fluid, or a solid tissue sample, such as a skin biopsy sample, skin strip, hair follicle, muscle biopsy sample, or alternatively a sample may be a buccal sample. Alternatively, a sample can comprise exosomes which may be harvested in order to be tested for FSGMs transcripts.

As use herein, the phrase “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

The term “such as” is used herein to mean, and is used interchangeably, with the phrase “such as but not limited to.”

A “transcribed polynucleotide” or “nucleotide transcript” is a polynucleotide (e.g. an mRNA, hnRNA, a cDNA, or an analog of such RNA or cDNA) which is complementary to or having a high percentage of identity (e.g., at least 80% identity) with all or a portion of a mature mRNA made by transcription of an FSGM of the invention and normal post-transcriptional processing (e.g. splicing), if any, of the RNA transcript, and reverse transcription of the RNA transcript.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Reference will now be made in detail to exemplary embodiments of the invention. While the invention will be described in conjunction with the exemplary embodiments, it will be understood that it is not intended to limit the invention to those embodiments. To the contrary, it is intended to cover alternatives, modifications, and equivalents as may be included within the spirit and scope of the invention as defined by the appended claims.

### C. FSGMs of the Invention

In one aspect, the present invention provides a method for determining, evaluating, and/or monitoring the effectiveness of FXN replacement therapy comprising determining: (i) a baseline FXN(−) expression profile for one or more FSGMs in a sample from an FXN deficient patient prior to treatment with FXN replacement therapy; and (ii) determining a patient FXN replacement expression profile for the FSGMs in a sample from an FXN deficient patient subsequent to treatment with FXN replacement therapy; comparing the patient FXN replacement expression profile with the baseline FXN(−) expression profile; and using the comparison to determine effectiveness of the FXN replacement therapy. Based on the results of the FSGM expression profile analysis, adjustments can be made to the FXN replacement therapy in the subject to, e.g., initiate, increase, decrease or cease FXN replacement therapy in the subject.

Another aspect of the disclosure relates to providing a method for identifying one or more FSGMs, which are markers whose expression is sensitive to FXN levels in a cell. The method comprises determining the expression profile in a sample from a healthy subject, having normal FXN levels, referred to herein as the normal FXN expression profile; determining the expression profile in a sample from a subject having deficient FXN levels, referred to herein as the baseline FXN(−) expression profile; and comparing the normal FXN expression profile with the baseline FXN(−) expression profile; wherein the markers whose expression is altered in the baseline FXN(−) expression profile compared to the normal FXN expression profile are the FSGMs. Additionally, or alternatively, the method for determining FSGMs may comprise the comparison between the expression profiles obtained from a sample from an FXN deficient subject before and after FXN replacement therapy. The gene expression profile from a sample from an FXN deficient subject after FXN replacement therapy is also referred to herein as an FXN replacement expression profile. By way of example, Table 2, Table 4 and FIG. 3 herein present FSGMs that were determined by a method of an embodiment of the disclosure.

The FSGMs of the invention include, but are not limited to any one or any combination of more than one of the FSGMs of Table 2, Table 4 and/or FIG. 3. In some embodiments of the present invention, other markers known in the art to measure FXN expression or FXN replacement therapy can be used in connection with the methods of the present invention.

As used herein, the term “one or more FSGMs” is intended to mean that one or more (e.g., 1, 2, 3, 4, 5, 6, 7, 8, 9, 10, 11, 12, 13, 14, 15, 16, 17, 18, 19, 20, or more) FSGMs is selected, e.g., from Table 2, Table 4 and/or FIG. 3. Methods, kits, and panels provided herein include one or any combination of, e.g., 1, 2, 3, 4, 5, 6, 7, 8, 9, 10, 11, 12, 13, 14, 15, 16, 17, 18, 19, 20, or more FSGMs selected from Table 2, Table 4 and/or FIG. 3.

In some embodiments, the one or more FSGMs of the invention comprise one or more (e.g., 1, 2, 3, 4, 5, 6, 7, 8, 9, 10, or more) secreted proteins, selected from Table 2, Table 4 and/or FIG. 3, i.e., a protein as set forth in Table 2 which is capable of being secreted by a cell. For example, the FSGM CYR61 is a secreted protein. Additional FSGMs that are secreted proteins include, for example, ADAMTS1, ASPN, FAM177A, IGF1, LOX, NRTN, SERPINE1, STC1, and THBS1. The expression levels of FSGMs that are secreted from cells can be measured using, for example, any suitable method for detecting polypeptide FSGMs of the invention or any protein detection method described herein. In certain embodiments, the detection method is an immunodetection method, e.g., ELISA, involving an antibody that specifically binds to one or more secreted protein, e.g., a secreted protein defined in Table 2.

In one embodiment, the one or more FSGMs comprise a secreted protein defined in Table 2, alone or in combination with one or more additional FSGM selected from Table 2, Table 4 and/or FIG. 3. In another embodiment, the one or more FSGMs comprise CYR61, alone or in combination with one or more additional FSGM selected from Table 2, Table 4 and/or FIG. 3. In another embodiment, the one or more FSGMs comprise one or more of CYR61, ADAMTS1, ASPN, FAM177A, IGF1, LOX, NRTN, SERPINE1, STC1, and/or THBS1, alone or in combination with one or more additional FSGM selected from Table 2, Table 4 and/or FIG. 3.

CYR61 is also referred to as Cellular Communication Network Factor 1 (CCN1), Insulin-Like Growth Factor-Binding Protein 10, Cysteine Rich Angiogenic Inducer 61, IGF-Binding Protein 10, CCN Family Member 1, Protein CYR61, IGFBP-10, IGFBP10, IBP-10, GIG1, Cysteine-Rich Heparin-Binding Protein 61, Cysteine-Rich, Anigogenic Inducer, 61. Cysteine-Rich Angiogenic inducer 61, and Protein GIG1. The secreted protein encoded by the CYR61 gene is growth factor-inducible and promotes the adhesion of endothelial cells. The protein interacts with several integrins and with heparan sulfate proteoglycan. It also acts as a linkage between SERPINE1, EGR2, NR4A1, and THBS and functions in the pathways of these genes. This protein also plays a role in cell proliferation, chemotaxis, angiogenesis, cell adhesion, differentiation, angiogenesis, apoptosis, and extracellular matrix formation. Diseases associated with CYR61 include Wilms Tumor 5 and Rhabdomyosarcoma. CYR61 can bind to α6 and β1 integrin heterodimers that have been shown to mediate schwann cell interactions with axons and facilitate axonal regeneration after peripheral nerve injury, hence potentially inhibiting this process (Chang et al., Neuroscience 2018, 371:49-59).

Exemplary GenBank Accession Nos. for the nucleotide and amino acid sequences of each of the FSGMs (or human homologs thereof) listed in Table 2 are set forth below in Table 4. These GenBank numbers are incorporated herein by reference in the versions available on the earliest effective filing date of this application. AI480526, C230034O21Rik, D130020L05Rik and Rpl37rt are mouse genes for which there is no human homologue, and therefore these genes do not appear in Table 4.

It is understood that the FSGMs of the invention include human homologues of the genes and proteins listed in Table 2.

TABLE 4
Exemplary GenBank Accession Numbers

| | NCBI | NCBI |
|---|---|---|
| | Accession | Accession |
| | No. for | No. for |
| Gene | mRNA | Protein |
| Symbol | sequence | sequence |
| Abce1 | NM_001040876.2 | NP_001035809.1 |
| Adamts1 | NM_006988.5 | NP_008919.3 |
| Adnp | NM_001282531.3 | NP_001269460.1 |
| Apold1 | NM_001130415.2 | NP_001123887.1 |
| Arc | NM_015193.5 | NP_056008.1 |
| Aspn | NM_001193335.2 | NP_001180264.1 |
| Atf3 | NM_001030287.3 | NP_001025458.1 |
| Bicd1 | NM_001003398.3 | NP_001003398.1 |
| Btg2 | NM_006763.3 | NP_006754.1 |
| Calm2 | NM_001305624.1 | NP_001292553.1 |
| Capza1 | NM_006135.3 | NP_006126.1 |
| Ccdc85b | NM_006848.3 | NP_006839.2 |
| Ccdc85c | NM_001144995.2 | NP_001138467.1 |
| Chm | NM_000390.4 | NP_000381.1 |
| Cops2 | NM_001143887.2 | NP_001137359.1 |
| Cript | NM_014171.6 | NP_054890.1 |
| Ctcfl | NM_001269040.1 | NP_001255969.1 |
| Ctss | NM_001199739.2 | NP_001186668.1 |
| Cul2 | NM_001198777.2 | NP_001185706.1 |
| Cycs | NM_018947.6 | NP_061820.1 |
| Cyr61 | NM_001554.5 | NP_001545.2 |
| Dclk1 | NM_001195415.1 | NP_001182344.1 |
| Dcun1d1 | NM_001308101.2 | NP_001295030.1 |
| Dfna5 | NM_001127453.2 | NP_001120925.1 |
| Dio2 | NM_000793.6 | NP_000784.3 |
| Dnajb9 | NM_012328.3 | NP_036460.1 |
| Dsel | NM_032160.3 | NP_115536.2 |
| Dynlt3 | NM_006520.3 | NP_006511.1 |
| Egr1 | NM_001964.3 | NP_001955.1 |
| Egr2 | NM_000399.5 | NP_000390.2 |
| Egr3 | NM_001199880.2 | NP_001186809.1 |
| EIF1AX | NM_001412.4 | NP_001403.1 |
| Emp1 | NM_001423.3 | NP_001414.1 |
| FAM177A1 | NM_001079519.1 | NP_001072987.1 |
| (C14ORF24) | NM_001289022.2 | NP_001275951.1 |
| | NM_173607.4 | NP_775878.2 |
| Gmfb | NM_004124.3 | NP_004115.1 |
| H4C13 | NM_003546.3 | NP_003537.1 |
| Igf1 | M_000618.5 | NP_000609.1 |
| KCTD12 | NM_138444.4 | NP_612453.1 |
| Lamp2 | NM_001122606.1 | NP_001116078.1 |
| Lamtor5 | NM_006402.2 | NP_006393.2 |
| Lox | NM_001178102.2 | NP_001171573.1 |
| Lypla1 | NM_001279356.1 | NP_001266285.1 |
| Lysmd3 | NM_001286812.1 | NP_001273741.1 |
| Maoa | NM_000240.4 | NP_000231.1 |
| Mki67 | NM_001145966.2 | NP_001139438.1 |
| Mob4 | NM_001100819.3 | NP_001094289.1 |
| Mpeg1 | NM_001039396.2 | NP_001034485.1 |
| Mt2a | NM_005953 | NP_005944 |
| mt-Atp6 | J01415 | YP_003024031.1 |
| mt-Atp8 | J01415 | YP_003024030.1 |
| mt-Co3 | J01415 | YP_003024032.1 |
| mt-Nd1 | J01415 | YP_003024026.1 |
| mt-Nd2 | J01415 | YP_003024027.1 |
| mt-Nd3 | J01415 | YP_003024033.1 |
| mt-Nd4 | J01415 | YP_003024035.1 |
| mt-Rnr1 | J01415 | |
| mt-Rnr2 | J01415 | |
| Nr4a1 | NM_001202233.1 | NP_001189162.1 |
| Nrtn | NM_004558.4 | NP_004549.1 |
| Orc4 | NM_001190879.2 | NP_001177808.1 |
| Pde4a | NM_001111307.2 | NP_001104777.1 |
| Pde4b | NM_001037339.2 | NP_001032416.1 |
| Phf1 | NM_002636.5 | NP_002627.2 |
| Psma3 | NM_002788.4 | NP_002779.1 |
| Ptgs2 | NM_000963.4 | NP_000954.1 |
| Ptp4a1 | NM_003463.4 | NP_003454.1 |
| Ptprc | NM_001267798.2 | NP_001254727.1 |
| Rap1b | NM_001010942.3 | NP_001010942.1 |
| Rap2c | NM_001271186.2 | NP_001258115.1 |
| Rnf13 | NM_001378285.1 | NP_001365214.1 |
| Rnf2 | NM_007212.4 | NP_009143.1 |
| Rpl10 | NM_001256577.2 | NP_001243506.2 |
| Rpl24 | NM_000986.4 | NP_000977.1 |
| Rpl26 | NM_000987.5 | NP_000978.1 |
| Rpl32 | NM_000994.4 | NP_000985.1 |
| Rpl38 | NM_000999.4 | NP_000990.1 |
| Rpl39 | NM_001000.4 | NP_000991.1 |
| Rps15a | NM_001019.5 | NP_001010.2 |
| Rps27l | NM_015920.4 | NP_057004.1 |
| Rtn4 | NM_001321859.2 | NP_001308788.1 |
| Serpine1 | NM_000602.5 | NP_000593.1 |
| Slirp | NM_001267863.1 | NP_001254792.1 |
| Spry4 | NM_001127496.3 | NP_001120968.1 |
| Stc1 | NM_003155.3 | NP_003146.1 |
| Suv420h2 | NM_032701.4 | NP_116090.2 |
| Thbs1 | NM_003246.4 | NP_003237.2 |
| Tmem126a | NM_001244735.1 | NP_001231664.1 |
| Top2a | NM_001067.4 | NP_001058.2 |
| Ube2d3 | NM_001300795.1 | NP_001287724.1 |
| Vbp1 | NM_001303543.1 | NP_001290472.1 |
| Wnk2 | NM_001282394.1 | NP_001269323.1 |
| Yam1 | | N/A |
| Yars | NM_003680.3 | NP_003671.1 |
| ZNF34 | NM_001286769.2 | NP_001273698.1 |
| | NM_001286770.1 | NP_001273699.1 |
| | NM_001378027.1 | NP_001364956.1 |
| | NM_001378028.1 | NP_001364957.1 |
| | NM_001378029.1 | NP_001364958.1 |
| | NM_030580.4 | NP_085057.3 |
| ZNF300 | NM_001172831.1 | NP_001166302.1 |
| | NM_001172832.1 | NP_001166303.1 |
| | NM_052860.2 | NP_443092.1 |
| Znrf1 | NM_032268.5 | NP_115644.1 |

In one embodiment, the one or more FSGMs comprise one or more of NR4A1, PTP4A1, ATF3, BTG2, EGR1, EGR2, EGR3, CYR61, and ABCE1. In another embodiment, the one or more FSGMs comprise one or more of EGR1, EGR2, EGR3 and IGF1. In another embodiment, the one or more FSGMs comprise one or more of MT-ND1, MT-ND2, MT-ND3, MT-ND4, MT-CO3, MT-ATP6, MT-ATP8, and CYCS. In another embodiment, the one or more FSGMs comprise one or more of OPS2, VBP1, PSMA3, SLIRP, CUL2, DCUN1D1, UBE2D3, ZNRF1, RNF2, and LAMP2. In another embodiment, the one or more FSGMs comprise one or more of RPS15A, EIF1AX, RPL24, RPL32, RPL26, RPL10, RPL39, RPL38, RPS27L, and ABCE1. In another embodiment, the one or more FSGMs comprise one or more of MT-ND1, MT-ND2, MT-ND3, MT-ND4, MT-CO3, and CYCS. In another embodiment, the one or more FSGMs comprise one or more of NR4A1, EGR1, EGR3, ADAMTS1, THBS1, SERPINE1, IGF1, PTGS2, and CYR61. In another embodiment, the one or more FSGMs comprise one or more of PSMA3, CUL2, UBE2D3, ZNRF1, RPS15A, RPL24, RPL32, RPL26, RPL10, RPL39, and RPL38. In another embodiment, the one or more FSGMs comprise one or more of ABCE1, RPS15A, EIF1AX, RPL24, RPL32, RPL26, RPL10, RPL39, and RPL38. In another embodiment, the one or more FSGMs comprise one or more of MT-ND1, MT-ND2, MT-ND3, MT-ND4, MT-CO3, MT-ATP6, MT-ATP8, CYCS, TMEM-126A, MAOA, and ABCE1. In another embodiment, the one or more FSGMs comprise one or more of MT-ND1, MT-ND2, MT-ND3, MT-ND4, MT-CO3, MT-ATP6, and MT-ATP8. In another embodiment, the one or more FSGMs comprise one or more of ABCE1, RPL26, RPL38, RPL10, RPL32, RPS15A, RPL24, RPL39, SLIRP, COPS2, DCUN1D1, RNF2, EGR1, BTG2, ATF3, PTGS2, IGF1, SERPINE1, and THBS1. In another embodiment, the one or more FSGMs comprise one or more of RPL26, THBS1, SERPINE1, IGF1, PTGS2, RPL10, RPS27L, CYCS, ATF3, BTG2, EGR1, EGR3, and CYR61.

By way of example, an FXN expression profile may be determined through the measurement of expression levels of at least one or any combination of more than one FSGM. As used herein, an FSGM includes any one or more of the FSGMs listed in Table 2, Table 4 and/or FIG. 3. An FSGM also includes any one of more of a gene encoding a secreted protein, e.g., a secreted protein defined in Table 2, e.g., CYR61, ADAMTS1, ASPN, FAM177A, IGF1, LOX, NRTN, SERPINE1, STC1, or THBS1, a mitochondrial gene, an EGR-family gene, insulin-like gene, ribosome depletion response gene, mitochondrial energy production gene, proteasome regulation gene, ribosomal function gene, respiratory chain gene, cardiac muscle development gene, macromolecule catabolism gene, translational initiation genes, mitochondrial components gene, oxidative phosphorylation gene, negative regulation of macromolecule metabolic process gene, and regulation of apoptotic process gene, or a protein encoded by any one of these genes.

Hereinafter an expression profile may also be referred to as a signature.

In one embodiment of the disclosure, a baseline FXN(−) expression profile may comprise an expression pattern exemplified in Table 2 by fold regulation in “KO (knockout) vs. WT (wild-type)” and/or in FIG. 3.

In one embodiment of the disclosure, a baseline FXN(−) expression profile comprises altered expression of at least one or any combination of more than one FSGM, e.g., any one or more of the FSGMs listed in Table 2, Table 4 and/or FIG. 3 or any one of more of a gene encoding a secreted protein, e.g., a secreted protein defined in Table 2, e.g., CYR61, ADAMTS1, ASPN, FAM177A, IGF1, LOX, NRTN, SERPINE1, STC1, or THBS1, a mitochondrial gene, an EGR-family gene, insulin-like gene, ribosome depletion response gene, mitochondrial energy production gene, proteasome regulation gene, ribosomal function gene, respiratory chain gene, cardiac muscle development gene, macromolecule catabolism gene, translational initiation genes, mitochondrial components gene, oxidative phosphorylation gene, negative regulation of macromolecule metabolic process gene, and regulation of apoptotic process gene, or a protein encoded by any one of these genes.

In another embodiment of the disclosure, a baseline FXN(−) expression profile may comprise the downregulated expression levels of at least one of ADNP, AI480526, C230034O21RIK, CCDCl85B, CCDCl85C, CTCFL, D130020L05RIK, mt-RNR1, mt-RNR2, NRTN, PDE4A, PHF1, RPL37RT, SLC26A10, SNORD17, SUV420H2, WNK2, YAM1, and/or ZNRF1, or any combination thereof. A measure of effectiveness of FXN replacement therapy may be indicated by a pattern of upregulation of any one or more of these FSGMs.

In another embodiment of the disclosure, a baseline FXN(−) expression profile may comprise the upregulated expression levels of CYR61. A measure of effectiveness of FXN replacement therapy may be indicated by a pattern of downregulation of CYR61.

In one embodiment, an FXN replacement expression profile comprises the reversed expression of a baseline FXN(−) expression profile.

In another embodiment, an FXN replacement expression profile for use as an indicator of FXN replacement treatment effectiveness may comprise one or any combination of two or more of the FSGMs presented in Table 2, Table 4 and/or FIG. 3, including, for example, a secreted protein such as CYR61, e.g., a secreted protein defined in Table 2, or one or more of CYR61, ADAMTS1, ASPN, FAM177A, IGF1, LOX, NRTN, SERPINE1, STC1, and THBS1, detected in a sample from a patient treated with FXN replacement therapy.

In another embodiment, an FXN replacement expression profile may comprise an expression pattern exemplified in Table 2 by fold regulation in “drug vs. vehicle”.

In some embodiments, an FXN replacement expression profile is characterized by the contrary regulation of FSGMs, which is defined by any FSGMs that were downregulated in FXN depletion conditions that become upregulated following FXN replacement therapy; and the reverse is also valid, such that any FSGMs that were upregulated in FXN depletion conditions become downregulated following FXN replacement therapy. Accordingly, detection of altered expression of one or more FSGMs in a sample following FXN replacement therapy allows for monitoring of efficacy of the FXN replacement therapy in a subject. For example, in one embodiment, a lack of altered expression of one or more FSGMs in a sample following FXN replacement therapy indicates that the FXN replacement therapy may not have been successful and/or that increased FXN replacement therapy may be needed. Likewise, in another embodiment, altered expression of one or more FSGMs in a sample following FXN replacement therapy indicates that FXN replacement therapy was successful.

In some embodiments, altered expression is modulated or altered gene expression, which in the method exemplified herein presents itself as differential gene expression, also known as differential mRNA expression. Altered or modulated expression may comprise increased expression, also referred to as overexpression or upregulation, or decreased or inhibited expression, also referred to as downregulation.

As referred to herein, feature vectors are a set of values that characterize an expression profile. Feature vectors may comprise a set of n FSGMs, n being the number of different genes whose expression levels were measured in a sample. By way of example, n may be all the FSGMs provided in Table 2, Table 4 and FIG. 3. Alternatively, n may be at least one, two, or three, or four, or five, or six, or any number of FSGMs presented in Table 2, Table 4 and FIG. 3, in any combination.

In one embodiment, a set of FSGMs may comprise at least one or any combination of more than one FSGM, e.g., any one or more of the FSGMs listed in Table 2, Table 4 and/or FIG. 3 or any one of more of a gene encoding a secreted protein, e.g., CYR61, ADAMTS1, ASPN, FAM177A, IGF1, LOX, NRTN, SERPINE1, STC1, or THBS1, a mitochondrial gene, EGR-family gene, insulin-like gene, ribosome depletion response gene, mitochondrial energy production gene, proteasome regulation gene, ribosomal function gene, respiratory chain gene, cardiac muscle development gene, macromolecule catabolism gene, translational initiation gene, mitochondrial components gene, oxidative phosphorylation gene, negative regulation of macromolecule metabolic process gene, or regulation of apoptotic process gene, or a protein encoded by any of these genes.

In one embodiment, the one or more FSGMs comprise CYR61. In another embodiment, the one or more FSGMs comprise one or more of CYR61, ADAMTS1, ASPN, FAM177A, IGF1, LOX, NRTN, SERPINE1, STC1, and THBS1. In one embodiment, the one or more FSGMs comprise one or more of NR4A1, PTP4A1, ATF3, BTG2, EGR1, EGR2, EGR3, CYR61, and ABCE1. In another embodiment, the one or more FSGMs comprise one or more of EGR1, EGR2, EGR3 and IGF1. In another embodiment, the one or more FSGMs comprise one or more of MT-ND1, MT-ND2, MT-ND3, MT-ND4, MT-CO2, MT-CO3, MT-ATP6, MT-ATP8, and CYCS. In another embodiment, the one or more FSGMs comprise one or more of OPS2, VBP1, PSMA3, SLIRP, CUL2, DCUN1D1, UBE2D3, ZNRF1, RNF2, and LAMP2. In another embodiment, the one or more FSGMs comprise one or more of RPS15A, EIF1AX, RPL24, RPL32, RPL26, RPL10, RPL39, RPL38, RPS27L, and ABCE1. In another embodiment, the one or more FSGMs comprise one or more of MT-ND1, MT-ND2, MT-ND3, MT-ND4, MT-CO3, and CYCS. In another embodiment, the one or more FSGMs comprise one or more of NR4A1, EGR1, EGR3, ADAMTS1, THBS1, SERPINE1, IGF1, PTGS2, and CYR61. In another embodiment, the one or more FSGMs comprise one or more of PSMA3, CUL2, UBE2D3, ZNRF1, RPS15A, RPL24, RPL32, RPL26, RPL10, RPL39, and RPL38. In another embodiment, the one or more FSGMs comprise one or more of ABCE1, RPS15A, EIF1AX, RPL24, RPL32, RPL26, RPL10, RPL39, and RPL38. In another embodiment, the one or more FSGMs comprise one or more of MT-ND1, MT-ND2, MT-ND3, MT-ND4, MT-CO3, MT-ATP6, MT-ATP8, CYCS, TMEM-126A, MAOA, and ABCE1. In another embodiment, the one or more FSGMs comprise one or more of MT-ND1, MT-ND2, MT-ND3, MT-ND4, MT-CO3, MT-ATP6, and MT-ATP8. In another embodiment, the one or more FSGMs comprise one or more of ABCE1, RPL26, RPL38, RPL10, RPL32, RPS15A, RPL24, RPL39, SLIRP, COPS2, DCUN1D1, RNF2, EGR1, BTG2, ATF3, PTGS2, IGF1, SERPINE1, and THBS1. In another embodiment, the one or more FSGMs comprise one or more of RPL26, THBS1, SERPINE1, IGF1, PTGS2, RPL10, RPS27L, CYCS, ATF3, BTG2, EGR1, EGR3, and CYR61.

By way of example, a normal FXN expression profile, obtained from samples of healthy subjects, may be comprised of expression levels of a set of FSGMs, and may be represented by and referred to as a normal FXN feature vector. As described in the following examples, FSGMs when measured in FXN deficient samples may present expression levels that are different from the levels of expression of FSGMs in healthy subjects, and thus may be represented by and referred to as a deficient FXN feature vector. In one embodiment, the difference between a deficient FXN feature vector and a normal FXN feature vector may be detected and quantified by the distance between the two feature vectors. In an alternative scenario expression levels of FSGMs from a sample from an FXN deficient patient following FXN replacement treatment may present yet different expression levels, and may be represented by and referred to as an FXN replacement feature vector. As for the previous two feature vectors, the difference between an FXN replacement feature vector and either a normal FXN feature vector or a deficient FXN feature vector may be detected and quantified by the distance between the replacement FXN feature vector and the normal FXN feature vector or the deficient FXN feature vector.

As such, having a sample from an FXN deficient patient obtained prior to treatment and a sample obtained post-FXN replacement treatment, a first FXN feature vector may be determined for the FXN replacement expression profile and a second FXN feature vector may be determined for the baseline FXN(−) expression profile; wherein determining a distance, or scalar product, between the first and the second feature vectors may be used for determining effectiveness of the FXN replacement therapy. In an embodiment of the disclosure, a third feature vector may be determined for the normal FXN expression profile, the normal expression profile being established for the FSGMs in a sample from a healthy subject. In an embodiment, the distance between the second (baseline FXN(−) expression profile) and third (normal FXN expression profile) FXN feature vectors may be determined. In another embodiment, the distance between the first (FXN replacement expression profile) and third (normal FXN expression profile) FXN feature vectors may be determined, and may be used for determining effectiveness of the FXN replacement therapy. In an embodiment, the distance between the first and third feature vectors may be normalized to the distance between the second and third feature vectors, and the resulting normalized distance may be used to determine effectiveness of the FXN replacement therapy. In an embodiment, the resulting normalized distance may be a value ranging from 0 (zero) to 1 (one), wherein the smaller the value (closest to zero) the more effective the therapy.

The markers of the invention, e.g., one or more FSGMs selected from Table 2, Table 4 and/or FIG. 3, are correlated with FXN levels in a subject. Accordingly, in one aspect, the present invention provides methods for using, measuring, detecting, quantifying, and the like of one or more of the FSGMs in Table 2, Table 4 and/or FIG. 3, e.g., CYR61, or one or more of CYR61, ADAMTS1, ASPN, FAM177A, IGF1, LOX, NRTN, SERPINE1, STC1, and THBS1, for determining and/or monitoring the FXN status in a subject or for determining, evaluating, and/or monitoring FXN replacement therapy in a subject.

In another aspect, the present invention relates to using, measuring, detecting, quantifying, and the like of one or more of the FSGMs in Table 2, Table 4 and/or FIG. 3, e.g., CYR61, CYR61, or one or more of CYR61, ADAMTS1, ASPN, FAM177A, IGF1, LOX, NRTN, SERPINE1, STC1, and THBS1, alone, or together with one or more additional FSGMs for FXN expression levels.

In addition, in another embodiment, the FSGMs may be used in combination with one or more additional markers for a mitochondrial disease, e.g., FRDA. Other markers that may be used in combination with the one or more FSGMs in Table 2, Table 4 and/or FIG. 3 include any measurable characteristic described herein that reflects in a quantitative or qualitative manner the physiological state of an organism, e.g., whether the organism has a mitochondrial disease, e.g., FRDA. The physiological state of an organism is inclusive of any disease or non-disease state, e.g., a subject having a mitochondrial disease, e.g., FRDA, or a subject who is otherwise healthy. The FSGMs of the invention that may be used in combination with the FSGMs in Table 2, Table 4 and/or FIG. 3 include characteristics that can be objectively measured and evaluated as indicators of normal processes, pathogenic processes, or pharmacologic responses to a therapeutic intervention. Such combination markers can be clinical parameters (e.g., age, performance status), laboratory measures (e.g., molecular markers), or genetic or other molecular determinants. In other embodiments, the present invention also involves the analysis and consideration of any clinical and/or patient-related health data, for example, data obtained from an Electronic Medical Record (e.g., collection of electronic health information about individual patients or populations relating to various types of data, such as, demographics, medical history, medication and allergies, immunization status, laboratory test results, radiology images, vital signs, personal statistics like age and weight, and billing information).

The present invention also contemplates the use of particular combinations of the FSGMs of Table 2, Table 4 and/or FIG. 3, e.g., combinations of FSGMs comprising CYR61 or combinations of FSGMs comprising one or more of CYR61, or one or more of CYR61, ADAMTS1, ASPN, FAM177A, IGF1, LOX, NRTN, SERPINE1, STC1, and THBS1, In one embodiment, the invention contemplates FSGM sets with at least two (2) members, which may include any two of the FSGMs in Table 2, Table 4 and/or FIG. 3. In another embodiment, the invention contemplates FSGM sets with at least three (3) members, which may include any three of the FSGMs in Table 2, Table 4 and/or FIG. 3. In another embodiment, the invention contemplates FSGM sets with at least four (4) members, which may include any four of the FSGMs in Table 2, Table 4 and/or FIG. 3. In another embodiment, the invention contemplates FSGM sets with at least five (5) members, which may include any five of the FSGMs in Table 2, Table 4 and/or FIG. 3. In another embodiment, the invention contemplates FSGM sets with at least six (6) members, which may include any six of the FSGMs in Table 2, Table 4 and/or FIG. 3. In another embodiment, the invention contemplates FSGM sets with at least seven (7) members, which may include any seven of the FSGMs in Table 2, Table 4 and/or FIG. 3. In another embodiment, the invention contemplates FSGM sets with at least eight (8) members, which may include any eight of the FSGMs in Table 2, Table 4 and/or FIG. 3. In another embodiment, the invention contemplates FSGM sets with at least nine (9) members, which may include any nine of the FSGMs in Table 2, Table 4 and/or FIG. 3. In another embodiment, the invention contemplates FSGM sets with at least ten (10) members, which may include any ten of the FSGMs in Table 2, Table 4 and/or FIG. 3. In another embodiment, the invention contemplates FSGM sets with at least eleven (11) members, which may include any ten of the FSGMs in Table 2, Table 4 and/or FIG. 3. In another embodiment, the invention contemplates FSGM sets with at least twelve (12) members, which may include any ten of the FSGMs in Table 2, Table 4 and/or FIG. 3. In other embodiments, the invention contemplates an FSGM set comprising at least 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, or 102 of the FSGMs listed in Table 2, Table 4 and/or FIG. 3. In one embodiment, the invention contemplates FSGM sets comprising at least 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, or 102 of the FSGMs listed in Table 2, Table 4 and/or FIG. 3, wherein one or more of the FSGMs in the set is a secreted protein, e.g., a secreted protein defined in Table 2, e.g., CYR61, ADAMTS1, ASPN, FAM177A, IGF1, LOX, NRTN, SERPINE1, STC1, and/or THBS1.

In another embodiment, the invention contemplates FSGM sets comprising at least 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, or 102 of the FSGMs listed in Table 2, Table 4 and/or FIG. 3, wherein one of the FSGMs in the set is CYR61.

In certain embodiments, the level of the FSGM is increased following treatment of a subject with FXN replacement, e.g., a subject deficient in FXN. In some embodiments, the FSGM is selected from the group consisting of mt-RNR1, mt-RNR2, ADNP, AI480526, C230034O21RIK, CCDCl85B, CCDCl85C, CTCFL, NRTN, PDE4A, PHF1, RPL37RT, SLC26A10, SNORD17, SUV420H2, WNK2, YAM1 and ZNRF1.

In other embodiments, the level of the FSGM is decreased following treatment of a subject with FXN replacement, e.g., a subject deficient in FXN. In some embodiments, the FSGM is selected from the group consisting of CYR61, mt-ATP6, mt-ATP8, mt-CO2, mt-CO3, mt-ND1, mt-ND2, mt-ND3 and mt-ND4, EGR1, EGR2, EGR3, IGF1, LAMP2, and SLIRP.

In another aspect, the present invention provides for the identification of a “diagnostic signature” or “diagnostic expression profile” based on the levels of the FSGMs of the invention in a biological sample, that correlates with FXN in the sample. The “levels of the FSGMs” can refer to the protein level of an FSGM in a biological sample. The “levels of the FSGMs” can also refer to the expression level of the genes corresponding to the proteins, e.g., by measuring the expression levels of the corresponding FSGM mRNAs. The collection or totality of levels of FSGMs provide a diagnostic signature that correlates with the level of FXN.

In certain embodiments, the diagnostic signature is obtained by (1) detecting the level of at least 1, 2, 3, 4, 5, 6, 7, 8, 9, 10 or more FSGMs in Table 2, Table 4 and/or FIG. 3, e.g., CYR61, ADAMTS1, ASPN, FAM177A, IGF1, LOX, NRTN, SERPINE1, STC1, and/or THBS1, in a biological sample from a subject receiving FXN replacement therapy (2) comparing the levels of the at least 1, 2, 3, 4, 5, 6, 7, 8, 9, 10 or more FSGMs in Table 2, Table 4 and/or FIG. 3, e.g., CYR61, ADAMTS1, ASPN, FAM177A, IGF1, LOX, NRTN, SERPINE1, STC1, and/or THBS1, to the levels of the same FSGMs from a control sample, such as a baseline FXN(−) expression profile, and (3) determining if the at least 1, 2, 3, 4, 5, 6, 7, 8, 9, 10 or more FSGMs in Table 2, Table 4 and/or FIG. 3, e.g., CYR61, ADAMTS1, ASPN, FAM177A, IGF1, LOX, NRTN, SERPINE1, STC1, and/or THBS1, detected in the biological sample are above or below the levels of the FSGMs in the control (e.g., baseline FXN(−) expression profile). If the at least 1, 2, 3, 4, 5, 6, 7, 8, 9, 10 or more FSGMs in Table 2, Table 4 and/or FIG. 3, e.g., CYR61, ADAMTS1, ASPN, FAM177A, IGF1, LOX, NRTN, SERPINE1, STC1, and/or THBS1, are above or below the control (e.g., baseline FXN(−) expression profile), then the diagnostic signature is indicative of the effectiveness of FXN replacement therapy.

In accordance with various embodiments, algorithms may be employed to predict whether or not a biological sample from a subject comprises FXN, or to evaluate or monitor whether the subject has effectively received FXN replacement therapy. The skilled artisan will appreciate that an algorithm can be any computation, formula, statistical survey, nomogram, look-up Tables, decision tree method, or computer program which processes a set of input variables (e.g., number of markers (n) which have been detected at a level exceeding some threshold level, or number of markers (n) which have been detected at a level below some threshold level) through a number of well-defined successive steps to eventually produce a score or “output.” Any suitable algorithm-whether computer-based or manual-based (e.g., look-up Tables)—is contemplated herein.

In certain embodiments, the FSGMs of the invention, e.g., CYR61, ADAMTS1, ASPN, FAM177A, IGF1, LOX, NRTN, SERPINE1, STC1, and/or THBS1, can include variant sequences. More particularly, certain binding agents/reagents used for detecting certain of the FSGMs of the invention can bind and/or identify variants of these certain FSGMs of the invention. As used herein, the term “variant” encompasses nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 80%, 85%, 90%, 95%, 96%, 97%, 98% or 99% identity to a sequence disclosed herein. The percentage identity is determined by aligning the two sequences to be compared as described below, determining the number of identical residues in the aligned portion, dividing that number by the total number of residues in the inventive (queried) sequence, and multiplying the result by 100.

Variant sequences generally differ from the specifically identified sequence only by conservative substitutions, deletions or modifications. As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Variants may also, or alternatively, contain other modifications, including the deletion or addition of amino acids that have minimal influence on the antigenic properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptide and polynucleotide sequences may be aligned, and percentages of identical amino acids or nucleotides in a specified region may be determined against another polypeptide or polynucleotide sequence, using computer algorithms that are publicly available. The percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity.

Two exemplary algorithms for aligning and identifying the identity of polynucleotide sequences are the BLASTN and FASTA algorithms. The alignment and identity of polypeptide sequences may be examined using the BLASTP algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia, Charlottesville, Va. 22906-9025. The FASTA algorithm, set to the default parameters described in the documentation and distributed with the algorithm, may be used in the determination of polynucleotide variants. The readme files for FASTA and FASTX Version 2.0× that are distributed with the algorithms describe the use of the algorithms and describe the default parameters.

The BLASTN software is available on the NCBI anonymous FTP server and is available from the National Center for Biotechnology Information (NCBI), National Library of Medicine, Building 38A, Room 8N805, Bethesda, Md. 20894. The BLASTN algorithm Version 2.0.6 [Sep. 10, 1998] and Version 2.0.11 [Jan. 20, 2000] set to the default parameters described in the documentation and distributed with the algorithm, is preferred for use in the determination of variants according to the present invention. The use of the BLAST family of algorithms, including BLASTN, is described at NCBI's website and in the publication of Altschul, et al., “Gapped BLAST and PSI-BLAST: a new generation of protein database search programs,” Nucleic Acids Res. 25:3389-3402, 1997.

In an alternative embodiment, variant polypeptides are encoded by polynucleotide sequences that hybridize to a disclosed polynucleotide under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

The invention provides for the use of various combinations and sub-combinations of FSGMs. For example, one or more secreted proteins, e.g., a secreted protein defined in Table 2, may be used in the methods of the invention, including CYR61, ADAMTS1, ASPN, FAM177A, IGF1, LOX, NRTN, SERPINE1, STC1, and/or THBS1. It is understood that any single FSGM or combination of the FSGMs provided herein can be used in the invention unless clearly indicated otherwise.

### D. Tissue Samples

The present invention may be practiced with any suitable biological sample that potentially contains, expresses, includes, a detectable FSGM. For example, the biological sample may be obtained from a body fluid sample such as blood (including any blood product, such as whole blood, plasma, serum, or specific types of cells of the blood), urine, saliva, or seminal fluid, or a solid tissue sample, such as a skin biopsy sample, muscle biopsy sample, or alternatively a sample may be a buccal sample. Alternatively, a sample can comprise exosomes which may be harvested in order to be tested for FSGM transcripts.

The inventive methods may be performed at the single cell level. However, the inventive methods may also be performed using a sample comprising many cells, where the assay is “averaging” expression over the entire collection of cells and tissue present in the sample. Preferably, there is enough of the tissue sample to accurately and reliably determine the expression levels of interest.

Any commercial device or system for isolating and/or obtaining tissue and/or blood or other biological products, and/or for processing said materials prior to conducting a detection reaction is contemplated.

In certain embodiments, the present invention relates to detecting FSGM nucleic acid molecules (e.g., mRNA encoding the protein FSGMs of Table 2, Table 4 and/or FIG. 3, for example, CYR61, ADAMTS1, ASPN, FAM177A, IGF1, LOX, NRTN, SERPINE1, STC1, and/or THBS1). In such embodiments, RNA can be extracted from a biological sample before analysis. Methods of RNA extraction are well known in the art (see, for example, J. Sambrook et al., “Molecular Cloning: A Laboratory Manual”, 1989, 2^{nd} Ed., Cold Spring Harbour Laboratory Press: New York). Most methods of RNA isolation from bodily fluids or tissues are based on the disruption of the tissue in the presence of protein denaturants to quickly and effectively inactivate RNases. Generally, RNA isolation reagents comprise, among other components, guanidinium thiocyanate and/or beta-mercaptoethanol, which are known to act as RNase inhibitors. Isolated total RNA is then further purified from the protein contaminants and concentrated by selective ethanol precipitations, phenol/chloroform extractions followed by isopropanol precipitation (see, for example, P. Chomczynski and N. Sacchi, Anal. Biochem., 1987, 162: 156-159) or cesium chloride, lithium chloride or cesium trifluoroacetate gradient centrifugations.

Numerous different and versatile kits can be used to extract RNA (i.e., total RNA or mRNA) from bodily fluids or tissues and are commercially available from, for example, Ambion, Inc. (Austin, Tex.), Amersham Biosciences (Piscataway, N.J.), BD Biosciences Clontech (Palo Alto, Calif.), BioRad Laboratories (Hercules, Calif.), GIBCO BRL (Gaithersburg, Md.), and Giagen, Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

In certain embodiments, after extraction, mRNA is amplified, and transcribed into cDNA, which can then serve as template for multiple rounds of transcription by the appropriate RNA polymerase. Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2nd Ed., Cold Spring Harbour Laboratory Press: New York; “Short Protocols in Molecular Biology”, F. M. Ausubel (Ed.), 2002, 5.sup.th Ed., John Wiley & Sons; U.S. Pat. Nos. 4,683,195; 4,683,202 and 4,800,159). Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as avian myeloblastosis virus reverse transcriptase or Moloney murine leukemia virus reverse transcriptase).

In certain embodiments, the RNA isolated from the tissue sample (for example, after amplification and/or conversion to cDNA or cRNA) is labeled with a detectable agent before being analyzed. The role of a detectable agent is to facilitate detection of RNA or to allow visualization of hybridized nucleic acid fragments (e.g., nucleic acid fragments hybridized to genetic probes in an array-based assay). Preferably, the detectable agent is selected such that it generates a signal which can be measured and whose intensity is related to the amount of labeled nucleic acids present in the sample being analyzed. In array-based analysis methods, the detectable agent is also preferably selected such that it generates a localized signal, thereby allowing spatial resolution of the signal from each spot on the array.

Methods for labeling nucleic acid molecules are well-known in the art. For a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachment of fluorescent dyes (see, for example, L. M. Smith et al., Nucl. Acids Res. 1985, 13: 2399-2412) or of enzymes (see, for example, B. A. Connoly and P. Rider, Nucl. Acids. Res. 1985, 13: 4485-4502); chemical modifications of nucleic acid fragments making them detectable immunochemically or by other affinity reactions (see, for example, T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232).

Any of a wide variety of detectable agents can be used in the practice of the present invention. Suitable detectable agents include, but are not limited to: various ligands, radionuclides, fluorescent dyes, chemiluminescent agents, microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

However, in some embodiments, the expression levels are determined by detecting the expression of a gene product (e.g., a protein, such as a secreted protein) thereby eliminating the need to obtain a genetic sample (e.g., RNA) from the sample.

### E. Detection and/or Measurement of FSGMs

Various methodologies may be utilized for measuring the distance between feature vectors. Once the data is normalized, the distance may be achieved for example by calculating the mean squared error, which may be extracted from the difference in the expression pattern of each gene measured in two different profiles, such as baseline FXN(−) and FXN replacement for example. Alternatively, the distance may be achieved by calculating a correlation coefficient or applying a t-test.

As described in detail herein, many methodologies have been described for the determination of RNA expression profiles, including sequencing, hybridization or amplification of the sample RNA. In a particular embodiment of the disclosure, said determining the expression profile of a sample of a patient comprises obtaining or provided a biological sample from a patient, extracting RNA from the sample, generating the corresponding cDNA, and detecting expression profile through any one of sequencing, hybridization or amplification.

Detecting the FXN-sensitive expression profiles by sequencing may use, for example, next generation sequencing (NGS), RNASeq, and any sequencing techniques known to the man skilled in the art.

Detecting expression profile by hybridization comprises contacting a patient sample, or a portion thereof, with a probe or a set of probes that specifically hybridize with FSGMs (or their transcripts) disclosed in Table 2, Table 4 and/or FIG. 3. In one embodiment of the disclosure, specific probes to at least one of the transcripts of genes encoding at least one of a secreted protein, e.g., a secreted protein defined in Table 2, e.g., CYR61, ADAMTS1, ASPN, FAM177A, IGF1, LOX, NRTN, SERPINE1, STC1, and/or THBS1, mitochondrial protein, EGR-family protein, insulin-like protein, ribosome depletion response protein, mitochondrial energy production protein, proteasome regulation protein, ribosomal function protein, respiratory chain protein, cardiac muscle development protein, macromolecule catabolism protein, translational initiation protein, mitochondrial components protein, oxidative phosphorylation protein, negative regulation of macromolecule metabolic process protein, regulation of apoptotic process protein, or any combination thereof, may be contacted with a patient sample. By way of example, specific probes for at least one of mt-ATP6, mt-ATP8, mt-CO2, mt-CO3, mt-ND1, mt-ND2, mt-ND3, mt-ND4, mt-RNR1, mt-RNR2, EGR1, EGR2, EGR3, IGF1, LAMP2, APOLD1, MAOA, PDE4A, YARS, RnF13 and RPL10, CYR61, ADAMTS1, ASPN, FAM177A, IGF1, LOX, NRTN, SERPINE1, STC1, and/or THBS1, or any combination thereof, may be contacted with a patient sample. Thus, determining the expression profile of a sample of a patient treated with FXN replacement therapy by hybridization may comprise contacting the sample, or a portion thereof, with probes that hybridize with at least 75%, 80%, 85%, 90%, 95%, 97%, 98% or 99% of its target nucleic acid, the target nucleic acid being the transcript, or the corresponding cDNA for any one of the FSGMs provided in Table 2, Table 4 and/or FIG. 3, e.g., CYR61, ADAMTS1, ASPN, FAM177A, IGF1, LOX, NRTN, SERPINE1, STC1, and/or THBS1.

Detecting expression profile by amplification involves, by way of example, polymerase chain reaction techniques, such as real-time polymerase chain reaction (RT-PCR), which comprises contacting the sample with forward and reverse primers for each of the transcripts of interest as exemplified herein below in the examples and generating RT-PCR products. Optionally RT-PCR products are detected with specific or general probes, or a combination thereof, which facilitate their quantification. Thus, an FXN-induced signature may be determined by detecting FSGMs transcripts in a sample. In an embodiment of the disclosure, forward and reverse primers are used for detecting FSGMs transcripts.

In an alternative embodiment the expression profile may be detected through FSGMs protein products and analysis of protein profile, which may be performed through protein detection methodology, using techniques involving specific antibodies, or protein quantification/characterization techniques, such as high-performance liquid chromatography (HPLC), mass spectrometry-based techniques, gel-based techniques for example differential in-gel electrophoresis, and the like.

In another aspect, the present disclosure provides a composition for detection of an FXN expression profile, the composition comprising at least one or a plurality of nucleotide sequences for detection of FSGMs. In one embodiment of the disclosure, the composition may be for detection of any one of an FXN replacement expression profile, a baseline FXN(−) expression profile and/or a normal FXN expression profile. The composition may comprise at least one nucleotide sequence for the detection of transcripts of the genes defined in Table 2, Table 4 and/or FIG. 3. The composition may comprise nucleotide sequences for the detection of two, three, four, five, six, seven, eight, nine, ten, fifteen, twenty, thirty, forty, and/or up to all FSGMs presented in Table 2, Table 4 and FIG. 3. By way of example, a composition for detection of an FXN signature comprises nucleotides for detection of at least one of mt-ATP6, mt-ATP8, mt-CO2, mt-CO3, mt-ND1, mt-ND2, mt-ND3, mt-ND4, mt-RnR1, mtRnR2, EGR1, EGR2, EGR3, IGF1, LAMP2, APOLD1, MAOA, PDE4A, YARS, RnF13, RPL10, SLIRP, CYR61, ADAMTS1, ASPN, FAM177A, IGF1, LOX, NRTN, SERPINE1, STC1, and/or THBS1, or any combination thereof. A nucleotide sequence may be DNA or an analog thereof, or RNA or an analog thereof. The nucleotide sequence may be complementary to as least a portion of an FSGM. Binding of the nucleotide sequence for detection of FSGMs will depend on the level of stringency of the reaction. The nucleotide sequence may be an oligonucleotide, which may function as a probe or a primer, and as such may comprise modifications compatible with their function. For example, short oligonucleotides used as probes may carry labels, for example fluorescent labels, that enable their detection and quantification.

The present invention contemplates any suitable means, techniques, and/or procedures for detecting and/or measuring the FSGMs of the invention. These methods are described in detail below.

1. Detection of Protein FSGMs

The present invention contemplates any suitable method for detecting polypeptide FSGMs of the invention, i.e., the proteins of Table 2, Table 4 and/or FIG. 3, including secreted proteins CYR61, ADAMTS1, ASPN, FAM177A, IGF1, LOX, NRTN, SERPINE1, STC1, and THBS1. In certain embodiments, the detection method is an immunodetection method involving an antibody that specifically binds to one or more of the FSGMs of Table 2, Table 4 and/or FIG. 3, e.g., CYR61, ADAMTS1, ASPN, FAM177A, IGF1, LOX, NRTN, SERPINE1, STC1, and THBS1. The steps of various useful immunodetection methods have been described in the scientific literature, such as, e.g., Nakamura et al. (1987), which is incorporated herein by reference.

In general, the immunobinding methods include obtaining a sample suspected of containing an FSGM protein, peptide, e.g., an FSGM secreted protein or peptide, or antibody, and contacting the sample, or a portion thereof, with an antibody or protein or peptide in accordance with the present invention, as the case may be, under conditions effective to allow the formation of immunocomplexes.

The immunobinding methods include methods for detecting or quantifying the amount of a reactive component in a sample, which methods require the detection or quantitation of any immune complexes formed during the binding process. Here, one would obtain a sample suspected of containing an FSGM protein, peptide or a corresponding antibody, and contact the sample with an antibody or encoded protein or peptide, as the case may be, and then detect or quantify the amount of immune complexes formed under the specific conditions.

Contacting the chosen biological sample, or a portion thereof, with the protein under conditions effective and for a period of time sufficient to allow the formation of immune complexes (primary immune complexes). Generally, complex formation is a matter of simply adding the composition to the biological sample and incubating the mixture for a period of time long enough for the antibodies to form immune complexes with, i.e., to bind to, any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or FSGM, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The protein employed in the detection may itself be linked to a detectable label, wherein one would then simply detect this label, thereby allowing the amount of the primary immune complexes in the composition to be determined.

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The immunodetection methods of the present invention have evident utility in the monitoring of efficacy of FXN replacement therapy, e.g., CTI-1601. Here, a biological or clinical sample suspected of containing either the encoded protein or peptide or corresponding antibody is used. However, these embodiments also have applications to non-clinical samples, such as in the titering of antigen or antibody samples, in the selection of hybridomas, and the like.

The present invention, in particular, contemplates the use of ELISAs as a type of immunodetection assay. It is contemplated that the FSGM proteins or peptides of the invention, including secreted proteins or peptides, e.g., a secreted protein defined in Table 2, such as CYR61, ADAMTS1, ASPN, FAM177A, IGF1, LOX, NRTN, SERPINE1, STC1, and/or THBS1, will find utility as immunogens in ELISA assays in monitoring of FXN replacement therapy. Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like also may be used.

In one exemplary ELISA, antibodies binding to the FSGMs of the invention, including secreted proteins, e.g., a secreted protein defined in Table 2, such as CYR61, ADAMTS1, ASPN, FAM177A, IGF1, LOX, NRTN, SERPINE1, STC1, and/or THBS1, are immobilized onto a selected surface exhibiting protein affinity, such as a well in a polystyrene microtiter plate. Then, a test composition suspected of containing the FSGM antigen, such as a clinical sample, is added to the wells. After binding and washing to remove non-specifically bound immunecomplexes, the bound antigen may be detected. Detection is generally achieved by the addition of a second antibody specific for the target protein, that is linked to a detectable label. This type of ELISA is a simple “sandwich ELISA.” Detection also may be achieved by the addition of a second antibody, followed by the addition of a third antibody that has binding affinity for the second antibody, with the third antibody being linked to a detectable label.

In another exemplary ELISA, the samples suspected of containing the FSGM antigen are immobilized onto the well surface and then contacted with the anti-biomarker antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Irrespective of the format employed, ELISAs have certain features in common, such as coating, incubating or binding, washing to remove non-specifically bound species, and detecting the bound immunecomplexes. These are described as follows.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control and/or clinical or biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

The phrase “under conditions effective to allow immunecomplex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25 to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this will be an enzyme that will generate color development upon incubating with an appropriate chromogenic substrate. Thus, for example, one will desire to contact and incubate the first or second immunecomplex with a urease, glucose oxidase, alkaline phosphatase or hydrogen peroxidase-conjugated antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple. Quantitation is then achieved by measuring the degree of color generation, e.g., using a visible spectra spectrophotometer.

The protein FSGMs of the invention, including secreted proteins such as CYR61, ADAMTS1, ASPN, FAM177A, IGF1, LOX, NRTN, SERPINE1, STC1, and/or THBS1, can also be measured, quantitated, detected, and otherwise analyzed using protein mass spectrometry methods and instrumentation. Protein mass spectrometry refers to the application of mass spectrometry to the study of proteins. Although not intending to be limiting, two approaches are typically used for characterizing proteins using mass spectrometry. In the first, intact proteins are ionized and then introduced to a mass analyzer. This approach is referred to as “top-down” strategy of protein analysis. The two primary methods for ionization of whole proteins are electrospray ionization (ESI) and matrix-assisted laser desorption/ionization (MALDI). In the second approach, proteins are enzymatically digested into smaller peptides using a protease such as trypsin. Subsequently these peptides are introduced into the mass spectrometer and identified by peptide mass fingerprinting or tandem mass spectrometry. Hence, this latter approach (also called “bottom-up” proteomics) uses identification at the peptide level to infer the existence of proteins.

Whole protein mass analysis of the FSGMs of the invention, including secreted proteins such as CYR61, ADAMTS1, ASPN, FAM177A, IGF1, LOX, NRTN, SERPINE1, STC1, and/or THBS1, can be conducted using time-of-flight (TOF) MS, or Fourier transform ion cyclotron resonance (FT-ICR). These two types of instruments are useful because of their wide mass range, and in the case of FT-ICR, its high mass accuracy. The most widely used instruments for peptide mass analysis are the MALDI time-of-flight instruments as they permit the acquisition of peptide mass fingerprints (PMFs) at high pace (1 PMF can be analyzed in approx. 10 sec). Multiple stage quadrupole-time-of-flight and the quadrupole ion trap also find use in this application.

The protein FSGMs of the invention, including secreted proteins, such as CYR61, ADAMTS1, ASPN, FAM177A, IGF1, LOX, NRTN, SERPINE1, STC1, and/or THBS1, can also be measured in complex mixtures of proteins and molecules that co-exist in a biological medium or sample, however, fractionation of the sample may be required and is contemplated herein. It will be appreciated that ionization of complex mixtures of proteins can result in situation where the more abundant proteins have a tendency to “drown” or suppress signals from less abundant proteins in the same sample. In addition, the mass spectrum from a complex mixture can be difficult to interpret because of the overwhelming number of mixture components. Fractionation can be used to first separate any complex mixture of proteins prior to mass spectrometry analysis. Two methods are widely used to fractionate proteins, or their peptide products from an enzymatic digestion. The first method fractionates whole proteins and is called two-dimensional gel electrophoresis. The second method, high performance liquid chromatography (LC or HPLC) is used to fractionate peptides after enzymatic digestion. In some situations, it may be desirable to combine both of these techniques. Any other suitable methods known in the art for fractionating protein mixtures are also contemplated herein.

Gel spots identified on a 2D Gel are usually attributable to one protein. If the identity of the protein is desired, usually the method of in-gel digestion is applied, where the protein spot of interest is excised, and digested proteolytically. The peptide masses resulting from the digestion can be determined by mass spectrometry using peptide mass fingerprinting. If this information does not allow unequivocal identification of the protein, its peptides can be subject to tandem mass spectrometry for de novo sequencing.

Characterization of protein mixtures using HPLC/MS may also be referred to in the art as “shotgun proteomics” and MuDPIT (Multi-Dimensional Protein Identification Technology). A peptide mixture that results from digestion of a protein mixture is fractionated by one or two steps of liquid chromatography (LC). The eluent from the chromatography stage can be either directly introduced to the mass spectrometer through electrospray ionization, or laid down on a series of small spots for later mass analysis using MALDI.

The protein FSGMs of the present invention, including secreted proteins such as CYR61, ADAMTS1, ASPN, FAM177A, IGF1, LOX, NRTN, SERPINE1, STC1, and/or THBS1, can be identified using MS using a variety of techniques, all of which are contemplated herein. Peptide mass fingerprinting uses the masses of proteolytic peptides as input to a search of a database of predicted masses that would arise from digestion of a list of known proteins. If a protein sequence in the reference list gives rise to a significant number of predicted masses that match the experimental values, there is some evidence that this protein was present in the original sample. It will be further appreciated that the development of methods and instrumentation for automated, data-dependent electrospray ionization (ESI) tandem mass spectrometry (MS/MS) in conjunction with microcapillary liquid chromatography (LC) and database searching has significantly increased the sensitivity and speed of the identification of gel-separated proteins. Microcapillary LC-MS/MS has been used successfully for the large-scale identification of individual proteins directly from mixtures without gel electrophoretic separation (Link et al., 1999; Opitek et al., 1997).

Several recent methods allow for the quantitation of proteins by mass spectrometry. For example, stable (e.g., non-radioactive) heavier isotopes of carbon (¹³C) or nitrogen (¹⁵N) can be incorporated into one sample while the other one can be labeled with corresponding light isotopes (e.g. ¹²C and ¹⁴N). The two samples are mixed before the analysis. Peptides derived from the different samples can be distinguished due to their mass difference. The ratio of their peak intensities corresponds to the relative abundance ratio of the peptides (and proteins). The most popular methods for isotope labeling are SILAC (stable isotope labeling by amino acids in cell culture), trypsin-catalyzed ¹⁸O labeling, ICAT (isotope coded affinity tagging), iTRAQ (isobaric tags for relative and absolute quantitation). “Semi-quantitative” mass spectrometry can be performed without labeling of samples. Typically, this is done with MALDI analysis (in linear mode). The peak intensity, or the peak area, from individual molecules (typically proteins) is here correlated to the amount of protein in the sample. However, the individual signal depends on the primary structure of the protein, on the complexity of the sample, and on the settings of the instrument. Other types of “label-free” quantitative mass spectrometry, uses the spectral counts (or peptide counts) of digested proteins as a means for determining relative protein amounts.

2. Detection of Nucleic Acids Corresponding to Protein FSGMs

In certain embodiments, the invention involves the detection of nucleic acid FSGMs, e.g., the corresponding genes or mRNA of the protein FSGMs of the invention, e.g., Table 2, Table 4 and/or FIG. 3, including CYR61, ADAMTS1, ASPN, FAM177A, IGF1, LOX, NRTN, SERPINE1, STC1, and THBS1.

In various embodiments, the methods of the present invention generally involve the determination of expression levels of a set of genes in a biological sample. Determination of gene expression levels in the practice of the inventive methods may be performed by any suitable method. For example, determination of gene expression levels may be performed by detecting the expression of mRNA expressed from the genes of interest and/or by detecting the expression of a polypeptide encoded by the genes.

For detecting nucleic acids encoding FSGMs of the invention, e.g., CYR61, ADAMTS1, ASPN, FAM177A, IGF1, LOX, NRTN, SERPINE1, STC1, and THBS1, any suitable method can be used, including, but not limited to, Southern blot analysis, Northern blot analysis, polymerase chain reaction (PCR) (see, for example, U.S. Pat. Nos. 4,683,195; 4,683,202, and 6,040,166; “PCR Protocols: A Guide to Methods and Applications”, Innis et al. (Eds), 1990, Academic Press: New York), reverse transcriptase PCR (RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320 308), one-sided PCR (Ohara et al., Proc. Natl. Acad. Sci., 1989, 86: 5673-5677), in situ hybridization, Taqman-based assays (Holland et al., Proc. Natl. Acad. Sci., 1991, 88: 7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems (see, for example, U.S. Pat. Nos. 5,409,818 and 5,554,527), Qbeta Replicase, Strand Displacement Amplification (SDA), Repair Chain Reaction (RCR), nuclease protection assays, subtraction-based methods, Rapid-Scan®, etc.

In other embodiments, gene expression levels of FSGMs of interest, e.g., CYR61, ADAMTS1, ASPN, FAM177A, IGF1, LOX, NRTN, SERPINE1, STC1, and THBS1, may be determined by amplifying complementary DNA (cDNA) or complementary RNA (cRNA) produced from mRNA and analyzing it using a microarray. A number of different array configurations and methods of their production are known to those skilled in the art (see, for example, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637). Microarray technology allows for the measurement of the steady-state mRNA level of a large number of genes simultaneously. Microarrays currently in wide use include cDNA arrays and oligonucleotide arrays. Analyses using microarrays are generally based on measurements of the intensity of the signal received from a labeled probe used to detect a cDNA sequence from the sample that hybridizes to a nucleic acid probe immobilized at a known location on the microarray (see, for example, U.S. Pat. Nos. 6,004,755; 6,218,114; 6,218,122; and 6,271,002). Array-based gene expression methods are known in the art and have been described in numerous scientific publications as well as in patents (see, for example, M. Schena et al., Science, 1995, 270: 467-470; M. Schena et al., Proc. Natl. Acad. Sci. USA 1996, 93: 10614-10619; J. J. Chen et al., Genomics, 1998, 51: 313-324; U.S. Pat. Nos. 5,143,854; 5,445,934; 5,807,522; 5,837,832; 6,040,138; 6,045,996; 6,284,460; and 6,607,885).

Nucleic acid used as a template for amplification can be isolated from cells contained in the biological sample, according to standard methodologies. (Sambrook et al., 1989) The nucleic acid may be genomic DNA or fractionated or whole cell RNA. Where RNA is used, it may be desired to convert the RNA to a complementary cDNA. In one embodiment, the RNA is whole cell RNA and is used directly as the template for amplification.

Pairs of primers that selectively hybridize to nucleic acids corresponding to any of the FSGM nucleotide sequences identified herein are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Affymax technology; Bellus, 1994). Following detection, one may compare the results seen in a given patient with a statistically significant reference group of, for example, normal patients. In this way, it is possible to correlate the amount of nucleic acid detected with various clinical states.

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-stranded or single-stranded form, although the single-stranded form is preferred.

A number of template dependent processes are available to amplify the nucleic acid sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety.

In PCR, two primer sequences are prepared which are complementary to regions on opposite complementary strands of the target nucleic acid sequence. An excess of deoxynucleoside triphosphates are added to a reaction mixture along with a DNA polymerase, e.g., Taq polymerase. If the target nucleic acid sequence is present in a sample, the primers will bind to the target nucleic acid and the polymerase will cause the primers to be extended along the target nucleic acid sequence by adding on nucleotides. By raising and lowering the temperature of the reaction mixture, the extended primers will dissociate from the target nucleic acid to form reaction products, excess primers will bind to the target nucleic acid and to the reaction products and the process is repeated.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641 filed Dec. 21, 1990. Polymerase chain reaction methodologies are well known in the art.

Another method for amplification is the ligase chain reaction (“LCR”), disclosed in European Application No. 320 308, incorporated herein by reference in its entirely. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[α-thio]-triphosphates in one strand of a restriction site also may be useful in the amplification of nucleic acids in the present invention. Walker et al. (1992), incorporated herein by reference in its entirety.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases may be added as biotinylated derivatives for easy detection. A similar approach is used in SDA. Target specific sequences also may be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Still other amplification methods described in GB Application No. 2 202 328, and in PCT Application No. PCT/US89/01025, each of which is incorporated herein by reference in its entirety, may be used in accordance with the present invention. In the former application, “modified” primers are used in a PCR like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other contemplated nucleic acid amplification procedures include transcription-based amplification systems (TAS), including nucleic acid sequence based amplification (NASBA) and 3SR. Kwoh et al. (1989); Gingeras et al., PCT Application WO 88/10315, incorporated herein by reference in their entirety. In NASBA, the nucleic acids may be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Davey et al., European Application No. 329 822 (incorporated herein by reference in its entirely) disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase 1), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA. These copies may then re-enter the cycle leading to very swift amplification. With proper choice of enzymes, this amplification may be done isothermally without addition of enzymes at each cycle. Because of the cyclical nature of this process, the starting sequence may be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR.” Frohman (1990) and Ohara et al. (1989), each herein incorporated by reference in their entirety.

Methods based on ligation of two (or more) oligonucleotides in the presence of nucleic acid having the sequence of the resulting “di-oligonucleotide”, thereby amplifying the di-oligonucleotide, also may be used in the amplification step of the present invention. Wu et al. (1989), incorporated herein by reference in its entirety.

Oligonucleotide probes or primers of the present invention may be of any suitable length, depending on the particular assay format and the particular needs and targeted sequences employed. In a preferred embodiment, the oligonucleotide probes or primers are at least 10 nucleotides in length (preferably, 10, 11, 12, 13, 14, 15, 16, 17, 18, 19, 20, 21, 22, 23, 24, 25, 26, 27, 28, 29, 30, 31, 32 . . . ) and they may be adapted to be especially suited for a chosen nucleic acid amplification system and/or hybridization system used. Longer probes and primers are also within the scope of the present invention as well known in the art. Primers having more than 30, more than 40, more than 50 nucleotides and probes having more than 100, more than 200, more than 300, more than 500 more than 800 and more than 1000 nucleotides in length are also covered by the present invention. Of course, longer primers have the disadvantage of being more expensive and thus, primers having between 12 and 30 nucleotides in length are usually designed and used in the art. As well known in the art, probes ranging from 10 to more than 2000 nucleotides in length can be used in the methods of the present invention. As for the % of identity described above, non-specifically described sizes of probes and primers (e.g., 16, 17, 31, 24, 39, 350, 450, 550, 900, 1240 nucleotides, . . . ) are also within the scope of the present invention.

In other embodiments, the detection means can utilize a hybridization technique, e.g., where a specific primer or probe is selected to anneal to a target FSGM of interest, and thereafter detection of selective hybridization is made. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1994, in Current Protocols in Molecular Biology, John Wiley & Sons Inc., N.Y.).

To enable hybridization to occur under the assay conditions of the present invention, oligonucleotide primers and probes should comprise an oligonucleotide sequence that has at least 70% (at least 71%, 72%, 73%, 74%), preferably at least 75% (75%, 76%, 77%, 78%, 79%, 80%, 81%, 82%, 83%, 84%, 85%, 86%, 87%, 88%, 89%) and more preferably at least 90% (90%, 91%, 92%, 93%, 94%, 95%, 96%, 97%, 98%, 99%, 100%) identity to a portion of an FSGM of the invention. Probes and primers of the present invention are those that hybridize under stringent hybridization conditions and those that hybridize to FSGM homologs of the invention under at least moderately stringent conditions. In certain embodiments probes and primers of the present invention have complete sequence identity to the FSGMs of the invention (gene sequences (e.g., cDNA or mRNA). It should be understood that other probes and primers could be easily designed and used in the present invention based on the FSGMs of the invention disclosed herein by using methods of computer alignment and sequence analysis known in the art (cf. Molecular Cloning: A Laboratory Manual, Third Edition, edited by Cold Spring Harbor Laboratory, 2000).

3. Antibodies and Labels

In some embodiments, the invention provides methods and compositions that include labels for the highly sensitive detection and quantitation of the FSGMs, e.g., CYR61, ADAMTS1, ASPN, FAM177A, IGF1, LOX, NRTN, SERPINE1, STC1, and THBS1, of the invention. One skilled in the art will recognize that many strategies can be used for labeling target molecules to enable their detection or discrimination in a mixture of particles. The labels may be attached by any known means, including methods that utilize non-specific or specific interactions of label and target. Labels may provide a detectable signal or affect the mobility of the particle in an electric field. In addition, labeling can be accomplished directly or through binding partners.

In some embodiments, the label comprises a binding partner that binds to the FSGM of interest, where the binding partner is attached to a fluorescent moiety. The compositions and methods of the invention may utilize highly fluorescent moieties, e.g., a moiety capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. Moieties suitable for the compositions and methods of the invention are described in more detail below.

In some embodiments, the invention provides a label for detecting a biological molecule comprising a binding partner for the biological molecule that is attached to a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the moiety comprises a plurality of fluorescent entities, e.g., about 2 to 4, 2 to 5, 2 to 6, 2 to 7, 2 to 8, 2 to 9, 2 to 10, or about 3 to 5, 3 to 6, 3 to 7, 3 to 8, 3 to 9, or 3 to 10 fluorescent entities. In some embodiments, the moiety comprises about 2 to 4 fluorescent entities. In some embodiments, the biological molecule is a protein or a small molecule. In some embodiments, the biological molecule is a protein. The fluorescent entities can be fluorescent dye molecules. In some embodiments, the fluorescent dye molecules comprise at least one substituted indolium ring system in which the substituent on the 3-carbon of the indolium ring contains a chemically reactive group or a conjugated substance. In some embodiments, the dye molecules are Alexa Fluor molecules selected from the group consisting of Alexa Fluor 488, Alexa Fluor 532, Alexa Fluor 647, Alexa Fluor 680 or Alexa Fluor 700. In some embodiments, the dye molecules are Alexa Fluor molecules selected from the group consisting of Alexa Fluor 488, Alexa Fluor 532, Alexa Fluor 680 or Alexa Fluor 700. In some embodiments, the dye molecules are Alexa Fluor 647 dye molecules. In some embodiments, the dye molecules comprise a first type and a second type of dye molecules, e.g., two different Alexa Fluor molecules, e.g., where the first type and second type of dye molecules have different emission spectra. The ratio of the number of first type to second type of dye molecule can be, e.g., 4 to 1, 3 to 1, 2 to 1, 1 to 1, 1 to 2, 1 to 3 or 1 to 4. The binding partner can be, e.g., an antibody.

In some embodiments, the invention provides a label for the detection of a biological FSGM of the invention, wherein the label comprises a binding partner for the FSGM and a fluorescent moiety, wherein the fluorescent moiety is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 3 microJoules. In some embodiments, the fluorescent moiety comprises a fluorescent molecule. In some embodiments, the fluorescent moiety comprises a plurality of fluorescent molecules, e.g., about 2 to 10, 2 to 8, 2 to 6, 2 to 4, 3 to 10, 3 to 8, or 3 to 6 fluorescent molecules. In some embodiments, the label comprises about 2 to 4 fluorescent molecules. In some embodiments, the fluorescent dye molecules comprise at least one substituted indolium ring system in which the substituent on the 3-carbon of the indolium ring contains a chemically reactive group or a conjugated substance. In some embodiments, the fluorescent molecules are selected from the group consisting of Alexa Fluor 488, Alexa Fluor 532, Alexa Fluor 647, Alexa Fluor 680 or Alexa Fluor 700. In some embodiments, the fluorescent molecules are selected from the group consisting of Alexa Fluor 488, Alexa Fluor 532, Alexa Fluor 680 or Alexa Fluor 700. In some embodiments, the fluorescent molecules are Alexa Fluor 647 molecules. In some embodiments, the binding partner comprises an antibody. In some embodiments, the antibody is a monoclonal antibody. In other embodiments, the antibody is a polyclonal antibody.

The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. An “antigen-binding fragment” of an antibody refers to the part of the antibody that participates in antigen binding. The antigen binding site is formed by amino acid residues of the N-terminal variable (“V”) regions of the heavy (“H”) and light (“L”) chains. It will be appreciated that the choice of epitope or region of the molecule to which the antibody is raised will determine its specificity, e.g., for various forms of the molecule, if present, or for total (e.g., all, or substantially all of the molecule).

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also commercially available (R and D Systems, Minneapolis, Minn.; HyTest, HyTest Ltd., Turku Finland; Abcam Inc., Cambridge, Mass., USA, Life Diagnostics, Inc., West Chester, Pa., USA; Fitzgerald Industries International, Inc., Concord, Mass. 01742-3049 USA; BiosPacific, Emeryville, Calif.).

In some embodiments, the antibody is a polyclonal antibody. In other embodiments, the antibody is a monoclonal antibody.

Antibodies may be prepared by any of a variety of techniques known to those of ordinary skill in the art (see, for example, Harlow and Lane, Antibodies: A Laboratory Manual, Cold Spring Harbor Laboratory, 1988). In general, antibodies can be produced by cell culture techniques, including the generation of monoclonal antibodies as described herein, or via transfection of antibody genes into suitable bacterial or mammalian cell hosts, in order to allow for the production of recombinant antibodies.

Monoclonal antibodies may be prepared using hybridoma methods, such as the technique of Kohler and Milstein (Eur. J. Immunol. 6:511-519, 1976), and improvements thereto. These methods involve the preparation of immortal cell lines capable of producing antibodies having the desired specificity. Monoclonal antibodies may also be made by recombinant DNA methods, such as those described in U.S. Pat. No. 4,816,567. DNA encoding antibodies employed in the disclosed methods may be isolated and sequenced using conventional procedures. Recombinant antibodies, antibody fragments, and/or fusions thereof, can be expressed in vitro or in prokaryotic cells (e.g. bacteria) or eukaryotic cells (e.g. yeast, insect or mammalian cells) and further purified as necessary using well known methods.

More particularly, monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner effective to stimulate antibody producing cells. The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

Antibodies may also be derived from a recombinant antibody library that is based on amino acid sequences that have been designed in silico and encoded by polynucleotides that are synthetically generated. Methods for designing and obtaining in silico-created sequences are known in the art (Knappik et al., J. Mol. Biol. 296:254:57-86, 2000; Krebs et al., J. Immunol. Methods 254:67-84, 2001; U.S. Pat. No. 6,300,064).

Digestion of antibodies to produce antigen-binding fragments thereof can be performed using techniques well known in the art. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment, which comprises both antigen-binding sites. “Fv” fragments can be produced by preferential proteolytic cleavage of an IgM, IgG or IgA immunoglobulin molecule, but are more commonly derived using recombinant techniques known in the art. The Fv fragment includes a non-covalent VH:: VL heterodimer including an antigen-binding site which retains much of the antigen recognition and binding capabilities of the native antibody molecule (Inbar et al., Proc. Natl. Acad. Sci. USA 69:2659-2662 (1972); Hochman et al., Biochem. 15:2706-2710 (1976); and Ehrlich et al., Biochem. 19:4091-4096 (1980)).

Antibody fragments that specifically bind to the protein FSGMs disclosed herein can also be isolated from a library of scFvs using known techniques, such as those described in U.S. Pat. No. 5,885,793.

A wide variety of expression systems are available in the art for the production of antibody fragments, including Fab fragments, scFv, VL and VHs. For example, expression systems of both prokaryotic and eukaryotic origin may be used for the large-scale production of antibody fragments. Particularly advantageous are expression systems that permit the secretion of large amounts of antibody fragments into the culture medium. Eukaryotic expression systems for large-scale production of antibody fragments and antibody fusion proteins have been described that are based on mammalian cells, insect cells, plants, transgenic animals, and lower eukaryotes. For example, the cost-effective, large-scale production of antibody fragments can be achieved in yeast fermentation systems. Large-scale fermentation of these organisms is well known in the art and is currently used for bulk production of several recombinant proteins.

Antibodies that bind to the protein FSGMs employed in the present methods are, in some cases, available commercially or can be obtained without undue experimentation.

In still other embodiments, particularly where oligonucleotides are used as binding partners to detect and hybridize to mRNAN FSGMs or other nucleic acid based FSGMs, the binding partners (e.g., oligonucleotides) can comprise a label, e.g., a fluorescent moiety or dye. In addition, any binding partner of the invention, e.g., an antibody, can also be labeled with a fluorescent moiety. The fluorescence of the moiety will be sufficient to allow detection in a single molecule detector, such as the single molecule detectors described herein. A “fluorescent moiety,” as that term is used herein, includes one or more fluorescent entities whose total fluorescence is such that the moiety may be detected in the single molecule detectors described herein. Thus, a fluorescent moiety may comprise a single entity (e.g., a Quantum Dot or fluorescent molecule) or a plurality of entities (e.g., a plurality of fluorescent molecules). It will be appreciated that when “moiety,” as that term is used herein, refers to a group of fluorescent entities, e.g., a plurality of fluorescent dye molecules, each individual entity may be attached to the binding partner separately or the entities may be attached together, as long as the entities as a group provide sufficient fluorescence to be detected.

Typically, the fluorescence of the moiety involves a combination of quantum efficiency and lack of photobleaching sufficient that the moiety is detectable above background levels in a single molecule detector, with the consistency necessary for the desired limit of detection, accuracy, and precision of the assay. For example, in some embodiments, the fluorescence of the fluorescent moiety is such that it allows detection and/or quantitation of a molecule, e.g., an FSGM, at a limit of detection of less than about 10, 5, 4, 3, 2, 1, 0.1, 0.01, 0.001, 0.00001, or 0.000001 pg/ml and with a coefficient of variation of less than about 20, 15, 14, 13, 12, 11, 10, 9, 8, 7, 6, 5, 4, 3, 2, 1% or less, e.g., about 10% or less, in the instruments described herein. In some embodiments, the fluorescence of the fluorescent moiety is such that it allows detection and/or quantitation of a molecule, e.g., an FSGM, at a limit of detection of less than about 5, 1, 0.5, 0.1, 0.05, 0.01, 0.005, 0.001 pg/ml and with a coefficient of variation of less than about 10%, in the instruments described herein. “Limit of detection,” or LoD, as those terms are used herein, includes the lowest concentration at which one can identify a sample as containing a molecule of the substance of interest, e.g., the first non-zero value. It can be defined by the variability of zeros and the slope of the standard curve. For example, the limit of detection of an assay may be determined by running a standard curve, determining the standard curve zero value, and adding 2 standard deviations to that value. A concentration of the substance of interest that produces a signal equal to this value is the “lower limit of detection” concentration.

Furthermore, the moiety has properties that are consistent with its use in the assay of choice. In some embodiments, the assay is an immunoassay, where the fluorescent moiety is attached to an antibody; the moiety must have properties such that it does not aggregate with other antibodies or proteins, or experiences no more aggregation than is consistent with the required accuracy and precision of the assay. In some embodiments, fluorescent moieties that are preferred are fluorescent moieties, e.g., dye molecules that have a combination of 1) high absorption coefficient; 2) high quantum yield; 3) high photostability (low photobleaching); and 4) compatibility with labeling the molecule of interest (e.g., protein) so that it may be analyzed using the analyzers and systems of the invention (e.g., does not cause precipitation of the protein of interest, or precipitation of a protein to which the moiety has been attached).

Any suitable fluorescent moiety may be used. Examples include, but are not limited to, Alexa Fluor dyes (Molecular Probes, Eugene, Oreg.). The Alexa Fluor dyes are disclosed in U.S. Pat. Nos. 6,977,305; 6,974,874; 6,130,101; and 6,974,305 which are herein incorporated by reference in their entirety. Some embodiments of the invention utilize a dye chosen from the group consisting of Alexa Fluor 647, Alexa Fluor 488, Alexa Fluor 532, Alexa Fluor 555, Alexa Fluor 610, Alexa Fluor 680, Alexa Fluor 700, and Alexa Fluor 750. Some embodiments of the invention utilize a dye chosen from the group consisting of Alexa Fluor 488, Alexa Fluor 532, Alexa Fluor 647, Alexa Fluor 700 and Alexa Fluor 750. Some embodiments of the invention utilize a dye chosen from the group consisting of Alexa Fluor 488, Alexa Fluor 532, Alexa Fluor 555, Alexa Fluor 610, Alexa Fluor 680, Alexa Fluor 700, and Alexa Fluor 750. Some embodiments of the invention utilize the Alexa Fluor 647 molecule, which has an absorption maximum between about 650 and 660 nm and an emission maximum between about 660 and 670 nm. The Alexa Fluor 647 dye is used alone or in combination with other Alexa Fluor dyes.

In some embodiments, the fluorescent label moiety that is used to detect an FSGM in a sample using the analyzer systems of the invention is a quantum dot. Quantum dots (QDs), also known as semiconductor nanocrystals or artificial atoms, are semiconductor crystals that contain anywhere between 100 to 1,000 electrons and range from 2-10 nm. Some QDs can be between 10-20 nm in diameter. QDs have high quantum yields, which makes them particularly useful for optical applications. QDs are fluorophores that fluoresce by forming excitons, which are similar to the excited state of traditional fluorophores, but have much longer lifetimes of up to 200 nanoseconds. This property provides QDs with low photobleaching. The energy level of QDs can be controlled by changing the size and shape of the QD, and the depth of the QDs' potential. One optical feature of small excitonic QDs is coloration, which is determined by the size of the dot. The larger the dot, the redder, or more towards the red end of the spectrum the fluorescence. The smaller the dot, the bluer or more towards the blue end it is. The bandgap energy that determines the energy and hence the color of the fluoresced light is inversely proportional to the square of the size of the QD. Larger QDs have more energy levels which are more closely spaced, thus allowing the QD to absorb photons containing less energy, i.e., those closer to the red end of the spectrum. Because the emission frequency of a dot is dependent on the bandgap, it is possible to control the output wavelength of a dot with extreme precision. In some embodiments the protein that is detected with the single molecule analyzer system is labeled with a QD. In some embodiments, the single molecule analyzer is used to detect a protein labeled with one QD and using a filter to allow for the detection of different proteins at different wavelengths.

### F. Isolated FSGMs

1. Isolated Polypeptide FSGMs

One aspect of the invention pertains to isolated FSGM proteins and biologically active portions thereof, including secreted proteins such as CYR61, ADAMTS1, ASPN, FAM177A, IGF1, LOX, NRTN, SERPINE1, STC1, and THBS1, as well as polypeptide fragments suitable for use as immunogens to raise antibodies directed against an FSGM protein or a fragment thereof. In one embodiment, the native FSGM protein can be isolated by an appropriate purification scheme using standard protein purification techniques. In another embodiment, a protein or peptide comprising the whole or a segment of the FSGM protein is produced by recombinant DNA techniques. Alternative to recombinant expression, such protein or peptide can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of an FSGM protein include polypeptides comprising amino acid sequences sufficiently identical to or derived from the amino acid sequence of the FSGM protein, which include fewer amino acids than the full length protein, and exhibit at least one activity of the corresponding full-length protein. Typically, biologically active portions comprise a domain or motif with at least one activity of the corresponding full-length protein. A biologically active portion of an FSGM protein of the invention can be a polypeptide which is, for example, 10, 25, 50, 100 or more amino acids in length. Moreover, other biologically active portions, in which other regions of the FSGM protein are deleted, can be prepared by recombinant techniques and evaluated for one or more of the functional activities of the native form of the FSGM protein.

Preferred FSGM proteins are listed in Table 2, Table 4 and/or FIG. 3. Other useful proteins are substantially identical (e.g., at least about 40%, preferably 50%, 60%, 70%, 80%, 90%, 91%, 92%, 93%, 94%, 95%, 96%, 97%, 98% or 99%) to one of these sequences and retain the functional activity of the corresponding naturally-occurring FSGM protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. Preferably, the percent identity between the two sequences is calculated using a global alignment. Alternatively, the percent identity between the two sequences is calculated using a local alignment. The percent identity between the two sequences is a function of the number of identical positions shared by the sequences (i.e., % identity=# of identical positions/total # of positions (e.g., overlapping positions)×100). In one embodiment the two sequences are the same length. In another embodiment, the two sequences are not the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTX programs of Altschul, et al. (1990) J. Mol. Biol. 215:403-410. BLAST nucleotide searches can be performed with the BLASTN program, score=100, wordlength=12 to obtain nucleotide sequences homologous to a nucleic acid molecules of the invention. BLAST protein searches can be performed with the BLASTP program, score=50, wordlength=3 to obtain amino acid sequences homologous to a protein molecules of the invention. To obtain gapped alignments for comparison purposes, a newer version of the BLAST algorithm called Gapped BLAST can be utilized as described in Altschul et al. (1997) Nucleic Acids Res. 25:3389-3402, which is able to perform gapped local alignments for the programs BLASTN, BLASTP and BLASTX. Alternatively, PSI-Blast can be used to perform an iterated search which detects distant relationships between molecules. When utilizing BLAST, Gapped BLAST, and PSI-Blast programs, the default parameters of the respective programs (e.g., BLASTX and BLASTN) can be used. See the NCBI website. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

2. Isolated Nucleic Acid FSGMs

One aspect of the invention pertains to isolated nucleic acid molecules which encode an FSGM protein or a portion thereof, e.g., a secreted protein or portion thereof. Isolated nucleic acids of the invention also include nucleic acid molecules sufficient for use as hybridization probes to identify FSGM nucleic acid molecules, and fragments of FSGM nucleic acid molecules, e.g., those suitable for use as PCR primers for the amplification of a specific product or mutation of FSGM nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. In one embodiment, an “isolated” nucleic acid molecule (preferably a protein-encoding sequences) is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. In another embodiment, an “isolated” nucleic acid molecule, such as a cDNA molecule, can be substantially free of other cellular material, or culture medium when produced by recombinant techniques, or substantially free of chemical precursors or other chemicals when chemically synthesized. A nucleic acid molecule that is substantially free of cellular material includes preparations having less than about 30%, 20%, 10%, or 5% of heterologous nucleic acid (also referred to herein as a “contaminating nucleic acid”).

A nucleic acid molecule of the present invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, nucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of an FSGM nucleic acid or to the nucleotide sequence of a nucleic acid encoding an FSGM protein. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises an FSGM nucleic acid or which encodes an FSGM protein. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 15, more preferably at least about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more FSGMs of the invention. In certain embodiments, the probes hybridize to nucleic acid sequences that traverse splice junctions. The probe comprises a label group attached thereto, e.g., a radioisotope, a fluorescent compound, an enzyme, or an enzyme co-factor. Such probes can be used as part of a diagnostic test kit or panel for identifying cells or tissues which express or mis-express the protein, such as by measuring levels of a nucleic acid molecule encoding the protein in a sample of cells from a subject, e.g., detecting mRNA levels or determining whether a gene encoding the protein or its translational control sequences have been mutated or deleted.

The invention further encompasses nucleic acid molecules that differ, due to degeneracy of the genetic code, from the nucleotide sequence of nucleic acids encoding an FSGM protein (e.g., protein having the sequence provided in the sequence listing), and thus encode the same protein.

It will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to an FSGM of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to an FSGM nucleic acid or to a nucleic acid encoding a marker protein. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

### G. Frataxin Replacement Therapy

The methodology provided in the present disclosure refers to the determination of a gene expression profile associated with FXN replacement therapy. FXN replacement therapy involves the administration of an FXN replacement therapeutic to a subject in need. A number of alternatives for delivery of exogenous FXN may be envisioned. The FXN replacement therapeutic may be provided by FXN protein delivery or through delivery of a nucleic acid encoding FXN. FXN protein delivery may be delivery of full length FXN or delivery of a FXN fusion protein.

As used herein, the term “FXN fusion protein” refers to FXN or a fragment of FXN fused to a full length or a fragment of a different protein, or to a peptide. In some embodiments, an FXN fusion protein comprises a polypeptide that comprises FXN, e.g., full-length hFXN (SEQ ID NO: 1) or mature hFXN (SEQ ID NO: 2). In some embodiments, the FXN fusion protein also comprises a cell penetrating peptide (CPP).

The term “cell penetrating peptide” or “CPP”, as used herein, refers to a short peptide sequence, typically between 5 and 30 amino acids long, that can facilitate cellular intake of various molecular cargo, such as proteins. Within the context of the present invention, a CPP present in an FXN fusion protein facilitates the delivery of the FXN fusion protein into a cell, e.g., a recipient cell. CPPs may be polycationic, i.e., have an amino acid composition that either contains a high relative abundance of positively charged amino acids, such as lysine or arginine. CCPs may also be amphipathic, i.e., have sequences that contain an alternating pattern of polar/charged amino acids and non-polar, hydrophobic amino acids. CPPs may also be hydrophobic, i.e., contain only apolar residues with low net charge, or have hydrophobic amino acid groups that are crucial for cellular uptake.

A CPP that may be comprised in the FXN fusion protein may be any CPP known to a person skilled in the art. For example, the CPP may be any CPP listed in the Database of Cell-Penetrating Peptides CPPsite 2.0, the entire contents of which are hereby incorporated herein by reference. For examples, CPPs useful in the context of the present invention may a cell penetrating peptide derived from a protein selected from the group consisting of HIV Trans-Activator of Transcription peptide (HIV-TAT), galanin, mastoparan, transportan, penetratin, polyarginine, VP22, transportan, amphipathic peptides such as MAP, KALA, ppTG20, proline-rich peptides, MPG-derived peptides, Pep-1, and also loligomers, arginine-rich peptides and calcitonin-derived peptides.

In some embodiments, the CPP comprises a TAT protein domain comprising amino acids 47-57 of the 86 amino acid full length HIV-TAT protein (which 11 amino acid peptide may also be referred to herein as “HIV-TAT”; SEQ ID NO:4). In one embodiment, the CPP consists of HIV-TAT (SEQ ID NO:4). In some embodiments, the CPP comprises amino acids 47-57 of the 86 amino acid full length HIV-TAT protein with a methionine added at the amino terminus for initiation (12 AA; “HIV-TAT+M”): MYGRKKRRQRRR (SEQ ID NO: 5). Table 5 below lists amino acid sequences of exemplary CPPs.

TABLE 5
Exemplary CPPs and corresponding
sequences

| SEQ | | |
|---|---|---|
| ID | | |
| NO. | CPP | Amino Acid Sequence |
| 4 | HIV-TAT | YGRKKRRQRRR |
| 5 | HIV-TAT+M | MYGRKKRRQRRR |
| 6 | Galanin | GWTLNSAGYLLGPHAVGNHRSFSDKNG |
| | | LTS |
| 7 | Mastoparan | INLKALAALAKKIL-NH₂ |
| 8 | Transportan | GWTLNSAGYLLGKINLKALAALAKKIL |
| 9 | Penetratin | RQIKIWFQNRRMKWKK |
| 10 | Polyarginine | RRRRRRRRR |
| 11 | VP22 | DAATATRGRSAASRPTERPRAPARSAS |
| | | RPRRPVE |

In some embodiments, the CPP comprised in the FXN fusion protein is HIV-TAT (SEQ ID NO: 4). In some embodiments, the FXN fusion protein comprises full-length FXN, e.g., SEQ ID NO: 1, and HIV-TAT, e.g., SEQ ID NO: 4, as CPP.

In some embodiments, in FXN fusion proteins of the present disclosure, the CPP may be fused together with the FXN, e.g., full-length FXN, via a linker to form a single polypeptide chain. Examples of FXN fusion proteins include TAT-FXN fusion proteins, where TAT or a fragment of TAT may be directly or indirectly (through a linker) linked to either the N- or the C-terminus of FXN. In one specific example, the linker may comprise the amino acid sequence GG.

In some aspects, the CPP, e.g., HIV-TAT, that is present in an FXN fusion protein of the present disclosure facilitates delivery of the FXN fusion protein into a cell, e.g., a cell that may be present in vitro, ex vivo, or in a subject. Once inside the cell, the FXN fusion protein may be processed by cellular machinery to remove the CPP, e.g., HIV-TAT, from the FXN.

One specific example of a TAT-FXN fusion protein is referred to as CTI-1601. CTI-1601 is a 24.9 kDa fusion protein currently under investigation as an FXN replacement therapy to restore functional levels of FXN in the mitochondria of FRDA patients. CTI-1601 includes the HIV-TAT peptide linked to the N-terminus of the full-length hFXN protein. CTI-1601 mechanism of action relies on the cell-penetrating ability of the HIV-TAT peptide to deliver the CTI-1601 into cells and the subsequent processing into mature hFXN after translocation into the mitochondria. CTI-1601 is described in U.S. Provisional Patent Application No. 62/880,073 and No. 62/891,029, the entire contents of each of which are hereby incorporated herein by reference. CTI-1601 comprises the following amino acid sequence (224 amino acids):

(SEQ ID NO: 12)
MYGRKKRRQRRRGGMWTLGRRAVAGLLASPSPAQAQTLTRVPRPAELAPL
CGRRGLRTDIDATCTPRRASSNQRGLNQIWNVKKQSVYLMNLRKSGTLGH
PGSLDETTYERLAEETLDSLAEFFEDLADKPYTFEDYDVSFGSGVLTVKL
GGDLGTYVINKQTPNKQIWLSSPSSGPKRYDWTGKNWVYSHDGVSLHELL
AAELTKALKTKLDLSSLAYSGKDA.

FXN replacement may also be delivered by viral gene replacement, which may utilize retroviral, lentiviral, and adeno-associated viral vectors, as well as adenoviruses. Alternatively, FXN replacement therapy may be achieved by upregulation of endogenous mutant FXN gene, which depending on the number of GAA repeats is expressed in varying levels in carriers of the mutant FXN allele.

### H. FSGM Applications

In some aspects, the invention provides methods for evaluating and/or monitoring the efficacy of FXN replacement therapy in a subject. The invention further provides methods for determining whether a subject is in need of FXN replacement therapy or a change in FXN replacement therapy, e.g., determining whether FXN replacement therapy should be initiated, increased, decreased or ceased in a subject. In some embodiments, the methods are carried out by the subject using a sample obtained from the same subject or as a point of care test, and results can be assessed by the subject or by a physician. In one aspect, the present invention constitutes an application of information obtainable by the methods of the invention in connection with analyzing, detecting, and/or measuring one or more of the FSGMs of the present invention, i.e., the FSGMs of Table 2, Table 4 and/or FIG. 3. In one embodiment, the one or more FSGM comprises a secreted protein, e.g., a secreted protein defined in Table 2. For example, in one embodiment, the one or more FSGM comprises CYR61. In another embodiment, the onen or more FSGM comprises one or more of CYR61, ADAMTS1, ASPN, FAM177A, IGF1, LOX, NRTN, SERPINE1, STC1, and THBS1.

For example, when executing the methods of the invention for detecting and/or measuring one or more protein FSGM of the present invention, i.e., the FSGMs of Table 2, Table 4 and/or FIG. 3, e.g., CYR61, ADAMTS1, ASPN, FAM177A, IGF1, LOX, NRTN, SERPINE1, STC1, and/or THBS1, as described herein, one may contact a biological sample with a detection reagent, e.g., a monoclonal antibody, which selectively binds to the FSGM of interest, forming a protein-protein complex, which is then further detected either directly (if the antibody comprises a label) or indirectly (if a secondary detection reagent is used, e.g., a secondary antibody, which in turn is labeled). Thus, the method of the invention transforms the polypeptide FSGMs of the invention, i.e., one or more of the FSGMs of Table 2, Table 4 and/or FIG. 3, e.g., CYR61, ADAMTS1, ASPN, FAM177A, IGF1, LOX, NRTN, SERPINE1, STC1, and/or THBS1, to a protein-protein complex that comprises either a detectable primary antibody or a primary and further secondary antibody. Forming such protein-protein complexes is required in order to identify the presence of the FSGM of interest and necessarily changes the physical characteristics and properties of the FSGM of interest as a result of conducting the methods of the invention.

The same principal applies when conducting the methods of the invention for detecting nucleic acids that correspond to one or more of the FSGMs of the invention, i.e., the FSGMs of Table 2, Table 4 and/or FIG. 3, e.g., CYR61, ADAMTS1, ASPN, FAM177A, IGF1, LOX, NRTN, SERPINE1, STC1, and/or THBS1. In particular, when amplification methods are used, the process results in the formation of a new population of amplicons, i.e., molecules that are newly synthesized and which were not present in the original biological sample, thereby physically transforming the biological sample. Similarly, when hybridization probes are used to detect a target FSGM, a physical new species of molecules is in effect created by the hybridization of the probes (optionally comprising a label) to the target biomarker mRNA (or other nucleic acid), which is then detected. Such polynucleotide products are effectively newly created or formed as a consequence of carrying out the methods of the invention.

Methods for monitoring or evaluating the efficacy of FXN replacement therapy in a subject over time are also provided. In these methods the amount of one or more FSGM, i.e., the FSGMs of Table 2, Table 4 and/or FIG. 3, e.g., CYR61, ADAMTS1, ASPN, FAM177A, IGF1, LOX, NRTN, SERPINE1, STC1, and/or THBS1, in a pair of samples (a first sample obtained from the subject at an earlier time point or prior to the treatment regimen and a second sample obtained from the subject at a later time point, e.g., at a later time point when the subject has undergone at least a portion of the treatment regimen) is assessed. It is understood that the methods of the invention include obtaining and analyzing more than two samples (e.g., 3, 4, 5, 6, 7, 8, 9, or more samples) at regular or irregular intervals for assessment of FSGM levels. Pairwise comparisons can be made between consecutive or non-consecutive subject samples. Trends of FSGM levels and rates of change of FSGM levels can be analyzed for any two or more consecutive or non-consecutive subject samples.

An exemplary method for detecting the presence or absence or change of expression level of an FSGM protein or a corresponding nucleic acid in a biological sample involves obtaining a biological sample from a subject and contacting the biological sample with a compound or an agent capable of detecting the polypeptide or nucleic acid (e.g., mRNA, genomic DNA, or cDNA). In some embodiments, the detection methods of the invention can thus be used to detect mRNA, protein, cDNA, or genomic DNA, for example, in the biological sample in vitro as well as in vivo.

Methods provided herein for detecting the presence, absence, change of expression level of an FSGM protein, e.g., a secreted protein, e.g., a secreted protein defined in Table 2, such as CYR61, ADAMTS1, ASPN, FAM177A, IGF1, LOX, NRTN, SERPINE1, STC1, and/or THBS1, or corresponding nucleic acid in a biological sample include obtaining a biological sample from a subject that may or may not contain the FSGM protein or nucleic acid to be detected, contacting the sample with an FSGM-specific binding agent (i.e., one or more FSGM-specific binding agents) that is capable of forming a complex with the FSGM protein or nucleic acid to be detected, and contacting the sample with a detection reagent for detection of the FSGM—FSGM-specific binding agent complex, if formed. It is understood that the methods provided herein for detecting an expression level of an FSGM in a biological sample includes the steps to perform the assay. In certain embodiments of the detection methods, the level of the FSGM protein or nucleic acid in the sample is none or below the threshold for detection.

The methods include formation of either a transient or stable complex between the FSGM and the FSGM-specific binding agent. The methods require that the complex, if formed, be formed for sufficient time to allow a detection reagent to bind the complex and produce a detectable signal (e.g., fluorescent signal, a signal from a product of an enzymatic reaction, e.g., a peroxidase reaction, a phosphatase reaction, a beta-galactosidase reaction, or a polymerase reaction).

In certain embodiments, all FSGMs are detected using the same method. In certain embodiments, all FSGMs are detected using the same biological sample (e.g., same body fluid or tissue). In certain embodiments, different FSGMs are detected using various methods. In certain embodiments, FSGMs are detected in different biological samples. In some embodiments, a biological sample is a body fluid sample such as blood (including any blood product, such as whole blood, plasma, serum, or specific types of cells of the blood), urine, saliva, or seminal fluid, or a solid tissue sample, such as a skin biopsy sample, skin strip, hair follicle, muscle biopsy sample, or a buccal sample.

FSGM levels can be detected based on the absolute expression level or a normalized or relative expression level. Detection of absolute FSGM levels may be preferable when monitoring the treatment of a subject or in determining if there is a change in the FXN status of a subject. For example, the expression level of one or more FSGMs can be monitored in a subject undergoing treatment with an FXN replacement therapy, e.g., at regular intervals, such a monthly intervals. A modulation in the level of one or more FSGMs can be monitored over time to observe trends in changes in FSGM levels. Expression levels of the FSGMs of the invention in the subject may be higher than the expression level of those FSGMs in a normal sample, but may be lower than the prior expression level, thus indicating a lack of efficacy of the FXN replacement therapy in the subject. Changes, or not, in FSGM levels may be more relevant to treatment decisions for the subject than FSGM levels present in the population. Rapid changes in FSGM levels in a subject may be indicative of an abnormal FXN levels, even if the FSGMs are within normal ranges for the population.

As an alternative to making determinations based on the absolute expression level of the FSGM, determinations may be based on the normalized expression level of the FSGM. Expression levels are normalized by correcting the absolute expression level of an FSGM by comparing its expression to the expression of a gene that is not an FSGM, e.g., a housekeeping gene that is constitutively expressed. Suitable genes for normalization include housekeeping genes such as the actin gene, or epithelial cell-specific genes. This normalization allows the comparison of the expression level in one sample, e.g., a sample from an FXN deficient subject, to another sample, e.g., a normal sample, or between samples from different sources.

The present disclosure describes a method for evaluating and/or monitoring effectiveness of treatment with FXN replacement therapy for a patient in need thereof, in which a sample from the patient is analyzed. As used herein, a sample may be a body fluid sample, such as a blood sample for example, or a solid tissue sample, such as a skin biopsy sample, muscle biopsy sample, or alternatively a sample may be a buccal sample. Essentially, a sample of any tissue or body fluid that comprises cells in which FXN expression profile may be analyzed may be used in any of the methods disclosed herein. Alternatively, exosomes may be harvested in order to be tested for FSGM transcripts.

As described in the Examples, the baseline FXN(−) expression profile was validated using cell-based systems in which FXN was downregulated and treatment with FXN-replacement therapy, such as an FXN fusion protein, demonstrated a contrary regulation of FSGMs.

Any one of the FXN expression profiles described herein may be part of one or more algorithms which may be used to analyze the FXN expression profile of a sample and determine whether the sample represents a sample of a normal subject, a sample from a patient pre-FXN replacement treatment or a sample from a patient post-FXN replacement treatment. The one or more algorithms may be used to analyze a sample from a patient treated with an FXN-replacement drug and determine whether the patient has reacted effectively to the treatment, and therefore expresses a profile characteristic of an FXN replacement expression profile or not.

Thus an algorithm for analyzing the expression profile of a sample may use any one of a baseline FXN(−) expression profile, an FXN replacement expression profile, or a normal FXN expression profile, or a combination of profiles. Where a sample having FXN signature expression patterns consistent with baseline FXN(−) expression profile represents lack of effectiveness of FXN replacement therapy; and the sample having FXN expression profile consistent with FXN replacement expression profile and/or normal FXN expression profile represents effectiveness of FXN replacement therapy. In an embodiment, a classifier may be applied to FXN expression profiles obtained from patient samples in order to obtain information about the samples, for example to characterize the status of the FXN expression profile, or to define whether the patient was administered FXN replacement therapy or not. Alternatively or additionally, a classifier may be applied for evaluating whether the FXN expression profile of the patient sample reached a certain threshold necessary for FXN replacement treatment to be considered effective.

Provided in the disclosure is also a method of treatment of a patient suffering from a mitochondrial disease having FXN deficiency, the method comprising determining an FXN expression profile in a sample from the patient, and comparing the FXN expression profile obtained from the sample with at least one of a normal FXN expression profile, a baseline FXN(−) expression profile, or an FXN replacement expression profile. The sample may be further classified as having a normal FXN, a baseline FXN(−) or an FXN replacement profile. Using the results of the comparison of the sample FXN profile with the FXN profiles described herein, a therapy regime using FXN replacement therapy may be initiated, paused or ceased. Alternatively, an FXN replacement therapy dosage regime may be modified, e.g., increased or decreased. In one embodiment, the method further comprises obtaining or providing a sample from a subject, e.g., a subject suffering from FXN deficiency.

In certain embodiments of the methods provided herein, an increase or decrease in the level of one or more FSGMs selected from Table 2, Table 4 and/or FIG. 3, e.g., a secreted protein, e.g., a secreted protein defined in Table 2, such as CYR61, ADAMTS1, ASPN, FAM177A, IGF1, LOX, NRTN, SERPINE1, STC1, and/or THBS1, in the biological sample as compared to the level of the one or more FSGMs in a control sample, e.g., a sample from a subject deficient for FXN, is an indication that the FXN replacement therapy is effective.

In certain embodiments of the methods provided herein, no increase or decrease in the detected expression level of one or more FSGMs selected from Table 2, Table 4 and/or FIG. 3, e.g., a secreted protein, e.g., a secreted protein defined in Table 2, such as CYR61, ADAMTS1, ASPN, FAM177A, IGF1, LOX, NRTN, SERPINE1, STC1, and/or THBS1, in the biological sample as compared to the expression level of the one or more FSGMs in a control sample, e.g., a sample from a subject deficient for FXN, is an indication that the FXN replacement therapy is ineffective, e.g., at the current dose, and should be modified.

In certain embodiments, the methods may also include monitoring a subject being administered FXN replacement therapy. In some embodiments, no increase or decrease in the detected expression level of one or more FSGMs selected from Table 2, Table 4 and/or FIG. 3, e.g., a secreted protein, e.g., a secreted protein defined in Table 2, such as CYR61, ADAMTS1, ASPN, FAM177A, IGF1, LOX, NRTN, SERPINE1, STC1, and/or THBS1, in a second sample obtained from a subject after FXN replacement therapy is administered to the subject, as compared to the level of the one or more FSGMs in a first sample obtained from the subject before FXN replacement therapy is administered to the subject, is an indication that the FXN replacement therapy is not efficacious and/or the subject is not responsive FXN replacement therapy. The method may further include the step of adjusting the FXN replacement therapy to a higher dose.

In other embodiments, an increased or decreased expression level of one or more FSGMs selected from Table 2, Table 4 and/or FIG. 3, e.g., a secreted protein such as CYR61, ADAMTS1, ASPN, FAM177A, IGF1, LOX, NRTN, SERPINE1, STC1, and/or THBS1, in a second sample obtained from a subject after FXN replacement therapy is administered to the subject, as compared to the expression level of the one or more FSGMs, e.g., a secreted protein such as CYR61, ADAMTS1, ASPN, FAM177A, IGF1, LOX, NRTN, SERPINE1, STC1, and/or THBS1, in a first sample obtained from the subject before FXN replacement therapy is administered, is an indication that the FXN replacement therapy is efficacious and/or the subject is responsive to the FXN replacement therapy. The method may further include the step of adjusting the FXN replacement therapy to a lower dose or ceasing the therapy.

In certain embodiments, the level of the FSGM is increased following treatment of a subject with an FXN replacement, e.g., a subject deficient in FXN. In some embodiments, the FSGM is selected from the group consisting of mt-RNR1, mt-RNR2, ADNP, AI480526, C230034O21RIK, CCDCl85B, CCDCl85C, CTCFL, NRTN, PDE4A, PHF1, RPL37RT, SLC26A10, SNORD17, SUV420H2, WNK2, YAM1 or ZNRF1.

In other embodiments, the level of the FSGM is decreased following treatment of a subject with an FXN replacement, e.g., a subject deficient in FXN. In some embodiments, the FSGM is selected from the group consisting of CYR61, mt-ATP6, mt-ATP8, mt-CO2, mt-CO3, mt-ND1, mt-ND2, mt-ND3 and mt-ND4, EGR1, EGR2, EGR3, IGF1, LAMP2, or SLIRP.

In other embodiments, the present invention also involves the analysis and consideration of any clinical and/or patient-related health data, for example, data obtained from an Electronic Medical Record (e.g., collection of electronic health information about individual patients or populations relating to various types of data, such as, demographics, medical history, medication and allergies, immunization status, laboratory test results, radiology images, vital signs, personal statistics like age and weight, and billing information).

In certain embodiments the methods provided herein further comprise obtaining a biological sample from a subject suspected of having a mitochondrial disease, e.g., FRDA.

In certain embodiments the methods provided herein further comprise selecting a treatment regimen for the subject based on the level of the one or more FSGMs selected from Table 2, Table 4 and/or FIG. 3. In certain embodiments, the treatment method is started, change, revised, or maintained based on the results from the methods of the invention, e.g., when it is determined that the subject is responding to the treatment regimen, or when it is determined that the subject is not responding to the treatment regimen, or when it is determined that the subject is insufficiently responding to the treatment regimen. In certain embodiments, the treatment method is changed based on the results from the methods.

In certain embodiments of the diagnostic and monitoring methods provided herein, the method further comprises isolating a component of the biological sample.

In certain embodiments of the diagnostic and monitoring methods provided herein, the method further comprises labeling a component of the biological sample.

In certain embodiments of the diagnostic and monitoring methods provided herein, the method further comprises amplifying a component of a biological sample.

In certain embodiments of the methods provided herein, the method comprises forming a complex with a probe and a component of a biological sample. In certain embodiments, forming a complex with a probe comprises forming a complex with at least one non-naturally occurring reagent. In certain embodiments of the methods provided herein, the method comprises processing the biological sample. In certain embodiments of the methods provided herein, the method of detecting a level of at least two FSGMs comprises a panel of FSGMs. In certain embodiments of the methods provided herein, the method of detecting a level comprises attaching the FSGM to be detected to a solid surface.

### I. Kits/Panels

The invention also provides compositions and kits for evaluating and monitoring effectiveness of FXN replacement therapy. In some embodiments, the kits of the disclosure may be used by a subject for self-evaluation or may be carried out by a subject for evaluation by a physician, or as point of care kits.

These kits may include one or more of the following: a reagent that specifically binds to an FSGM of the invention, and a set of instructions for measuring the level of the FSGM. In one embodiment, the FSGM comprises a secreted protein such as CYR61, ADAMTS1, ASPN, FAM177A, IGF1, LOX, NRTN, SERPINE1, STC1, or THBS1. In another embodiment, the FSGM comprises CYR61.

The invention also encompasses kits for detecting the presence of an FSGM protein or nucleic acid in a biological sample. Such kits can be used to evaluate and/or monitor effectiveness of FXN replacement therapy. For example, the kit can comprise a labeled compound or agent capable of detecting an FSGM protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for use of the kit for practicing any of the methods provided herein or interpreting the results obtained using the kit based on the teachings provided herein. The kits can also include reagents for detection of a control protein in the sample, e.g., actin for tissue samples, albumin in blood or blood derived samples for normalization of the amount of the FSGM present in the sample. The kit can also include the purified FSGM for detection for use as a control or for quantitation of the assay performed with the kit. In some embodiments, a biological sample which is evaluated by a kit or panel of the disclosure is a body fluid sample such as blood (including any blood product, such as whole blood, plasma, serum, or specific types of cells of the blood), urine, saliva, or seminal fluid, or a solid tissue sample, such as a skin biopsy sample, skin strip, hair follicle, muscle biopsy sample, or a buccal sample.

Kits include a panel of reagents for use in a method to evaluate and/or monitor effectiveness of FXN replacement therapy, the panel comprising at least two detection reagents, wherein each detection reagent is specific for one FSGM, wherein said FSGMs are selected from the FSGM protein sets provided herein. In one embodiment, the FSGM comprises a secreted protein such as CYR61, ADAMTS1, ASPN, FAM177A, IGF1, LOX, NRTN, SERPINE1, STC1, or THBS1. In another embodiment, the FSGM comprises CYR61.

For antibody-based kits, the kit can comprise, for example: (1) a first antibody (e.g., attached to a solid support) which binds to a first FSGM protein; and, optionally, (2) a second, different antibody which binds to either the first FSGM protein or the first antibody and is conjugated to a detectable label. In certain embodiments, the kit includes (1) a second antibody (e.g., attached to a solid support) which binds to a second FSGM protein; and, optionally, (2) a third, different antibody which binds to either the second FSGM protein or the second antibody and is conjugated to a detectable label. The first and second FSGM proteins are different. In an embodiment, the first and second FSGMs are FSGMs of the invention, e.g., one or more of the FSGMs selected from Table 2, Table 4 and/or FIG. 3. In certain embodiments, the kit comprises a third antibody which binds to a third FSGM protein which is different from the first and second FSGM proteins, and a fourth different antibody that binds to either the third FSGM protein or the antibody that binds the third FSGM protein wherein the third FSGM protein is different from the first and second FSGM proteins.

For oligonucleotide-based kits, the kit can comprise, for example: (1) an oligonucleotide, e.g., a detectably labeled oligonucleotide, which hybridizes to a nucleic acid sequence encoding an FSGM protein or (2) a pair of primers useful for amplifying an FSGM nucleic acid molecule. In certain embodiments, the kit can further include, for example: (1) an oligonucleotide, e.g., a second detectably labeled oligonucleotide, which hybridizes to a nucleic acid sequence encoding a second FSGM protein or (2) a pair of primers useful for amplifying the second FSGM nucleic acid molecule. The first and second FSGMs are different. In an embodiment, the first and second FSGMs are FSGMs of the invention, e.g., one or more of the FSGMs selected from Table 2, Table 4 and/or FIG. 3. In certain embodiments, the kit can further include, for example: (1) an oligonucleotide, e.g., a third detectably labeled oligonucleotide, which hybridizes to a nucleic acid sequence encoding a third FSGM protein or (2) a pair of primers useful for amplifying the third FSGM nucleic acid molecule wherein the third FSGM is different from the first and second FSGMs. In certain embodiments, the kit includes a third primer specific for each nucleic acid FSGM to allow for detection using quantitative PCR methods.

For chromatography methods, the kit can include FSGMs, including labeled FSGMs, to permit detection and identification of one or more FSGMs of the invention, e.g., one or more of the FSGMs selected from Table 2, Table 4 and/or FIG. 3, by chromatography. In certain embodiments, kits for chromatography methods include compounds for derivatization of one or more FSGMs of the invention. In certain embodiments, kits for chromatography methods include columns for resolving the FSGMs of the method.

Reagents specific for detection of an FSGM of the invention, e.g., one or more of the FSGMs selected from Table 2, Table 4 and/or FIG. 3, allow for detection and quantitation of the FSGM in a complex mixture, e.g., cell or tissue sample. In certain embodiments, the reagents are species specific. In certain embodiments, the reagents are not species specific. In certain embodiments, the reagents are isoform specific. In certain embodiments, the reagents are not isoform specific.

In certain embodiments, the kits for evaluation and/or monitoring of the effectiveness of FXN replacement therapy comprise at least one reagent specific for the detection of the level of one or more of the FSGMs selected from Table 2, Table 4 and/or FIG. 3. In certain embodiments, the kits further comprise instructions for the detection, evaluation and/or monitoring of the effectiveness of FXN replacement therapy based on the level of the at least one FSGM selected from Table 2, Table 4 and/or FIG. 3.

The invention provides kits comprising at least one reagent specific for the detection of a level of at least one FSGM selected from Table 2, Table 4 and/or FIG. 3. In one embodiment, the FSGM comprises a secreted protein such as CYR61, ADAMTS1, ASPN, FAM177A, IGF1, LOX, NRTN, SERPINE1, STC1, or THBS1. In another embodiment, the FSGM comprises CYR61.

In certain embodiments, the kits can also comprise, e.g., a buffering agents, a preservative, a protein stabilizing agent, reaction buffers. The kit can further comprise components necessary for detecting the detectable label (e.g., an enzyme or a substrate). The kit can also contain a control sample or a series of control samples which can be assayed and compared to the test sample. The controls can be control serum samples or control samples of purified proteins or nucleic acids, as appropriate, with known levels of target FSGMs. Each component of the kit can be enclosed within an individual container and all of the various containers can be within a single package, along with instructions for interpreting the results of the assays performed using the kit.

The kits of the invention may optionally comprise additional components useful for performing the methods of the invention.

The invention further provides panels of reagents for detection of one or more FSGM in a subject sample and at least one control reagent. In certain embodiments, the FSGM comprises at least two or more FSGMs, wherein each of the two or more FSGMs are selected from Table 2, Table 4 and/or FIG. 3. In one embodiment, the one or more FSGM comprises a secreted protein, e.g., a secreted protein defined in Table 2, such as CYR61, ADAMTS1, ASPN, FAM177A, IGF1, LOX, NRTN, SERPINE1, STC1, and/or THBS1. In another embodiment, the one or more FSGM comprises CYR61.

In certain embodiments, the control reagent is to detect the FSGM for detection in the biological sample wherein the panel is provided with a control sample containing the FSGM for use as a positive control and optionally to quantitate the amount of FSGM present in the biological sample. The panel can be provided with reagents for detection of a control protein, e.g., actin for tissue samples, albumin in blood or blood derived samples for normalization of the amount of the FSGM present in the sample. The panel can be provided with a purified FSGM for detection for use as a control or for quantitation of the assay performed with the panel.

In certain embodiments, the level of the FSGM in the panel is increased when compared to a control or in a subject following administration of an FXN replacement, e.g., a subject deficient in FXN. In some embodiments, the FSGM is selected from the group consisting of mt-RNR1, mt-RNR2, ADNP, AI480526, C230034O21RIK, CCDCl85B, CCDCl85C, CTCFL, NRTN, PDE4A, PHF1, RPL37RT, SLC26A10, SNORD17, SUV420H2, WNK2, YAM1 or ZNRF1.

In certain embodiments, the level of the FSGM in the panel is decreased when compared to a control or in a subject following administration of an FXN replacement, e.g., a subject deficient in FXN. In some embodiments, the FSGM is selected from the group consisting of CYR61, mt-ATP6, mt-ATP8, mt-CO2, mt-CO3, mt-ND1, mt-ND2, mt-ND3 and mt-ND4, EGR1, EGR2, EGR3, IGF1, LAMP2, or SLIRP.

In some embodiments, the panel comprises one or more FSGMs with an increased level when compared to a control following treatment of a subject with FXN replacement, e.g., a subject deficient in FXN, and/or one or more FSGMs with a decreased level when compared to a control or following treatment of a subject with FXN replacement, e.g., a subject deficient in FXN.

In a preferred embodiment, the panel includes reagents for detection of two or more FSGMs of the invention (e.g., 2, 3, 4, 5, 6, 7, 8, 9 or up to all of the FSGMs recited in Table 2, Table 4 and/or FIG. 3), preferably in conjunction with a control reagent. In some embodiments, the panel includes reagents for detection of CYR61; in some embodiments, the panel includes reagents for detection of one or more of CYR61, ADAMTS1, ASPN, FAM177A, IGF1, LOX, NRTN, SERPINE1, STC1, and THBS1; in some embodiments, the panel includes reagents for detection of one or more of NR4A1, PTP4A1, ATF3, BTG2, EGR1, EGR2, EGR3, CYR61, and ABCE1; one or more of EGR1, EGR2, EGR3 and IGF1; one or more of MT-ND1, MT-ND2, MT-ND3, MT-ND4, MT-CO3, MT-ATP6, MT-ATP8, and CYCS; one or more of OPS2, VBP1, PSMA3, SLIRP, CUL2, DCUN1D1, UBE2D3, ZNRF1, RNF2, and LAMP2; one or more of RPS15A, EIF1AX, RPL24, RPL32, RPL26, RPL10, RPL39, RPL38, RPS27L, and ABCE1; one or more of MT-ND1, MT-ND2, MT-ND3, MT-ND4, MT-CO3, and CYCS; one or more of NR4A1, EGR1, EGR3, ADAMTS1, THBS1, SERPINE1, IGF1, PTGS2, and CYR61; one or more of PSMA3, CUL2, UBE2D3, ZNRF1, RPS15A, RPL24, RPL32, RPL26, RPL10, RPL39, and RPL38; one or more of ABCE1, RPS15A, EIF1AX, RPL24, RPL32, RPL26, RPL10, RPL39, and RPL38; one or more of MT-ND1, MT-ND2, MT-ND3, MT-ND4, MT-CO3, MT-ATP6, MT-ATP8, CYCS, TMEM-126A, MAOA, and ABCE1; one or more of MT-ND1, MT-ND2, MT-ND3, MT-ND4, MT-CO3, MT-ATP6, and MT-ATP8; one or more of ABCE1, RPL26, RPL38, RPL10, RPL32, RPS15A, RPL24, RPL39, SLIRP, COPS2, DCUN1D1, RNF2, EGR1, BTG2, ATF3, PTGS2, IGF1, SERPINE1, and THBS1; or one or more of RPL26, THBS1, SERPINE1, IGF1, PTGS2, RPL10, RPS27L, CYCS, ATF3, BTG2, EGR1, EGR3, and CYR61.

In the panel, each FSGM is detected by a reagent specific for that FSGM. In certain embodiments, the panel includes replicate wells, spots, or portions to allow for analysis of various dilutions (e.g., serial dilutions) of biological samples and control samples. In a preferred embodiment, the panel allows for quantitative detection of one or more FSGMs of the invention.

In certain embodiments, the panel is a protein chip for detection of one or more FSGMs. In certain embodiments, the panel is an ELISA plate for detection of one or more FSGMs. In certain embodiments, the panel is a plate for quantitative PCR for detection of one or more FSGMs.

In certain embodiments, the panel of detection reagents is provided on a single device including a detection reagent for one or more FSGMs of the invention and at least one control sample. In certain embodiments, the panel of detection reagents is provided on a single device including a detection reagent for two or more FSGMs of the invention and at least one control sample. In certain embodiments, multiple panels for the detection of different FSGMs of the invention are provided with at least one uniform control sample to facilitate comparison of results between panels.

### EXAMPLES

### Example 1: Generation of an FXN-Induced Signature

FXN Fusion Protein

The FXN fusion protein used in this Example is a fusion protein comprising TAT-cpp and hFXN linked through a linker at the N-terminus of hFXN (Vyas et al. (2012) Hum. Mol. Genet. 21, 1230-1247), referred to herein as CTI-1601. The hFXN in the fusion protein is the full-length 210 aa frataxin long precursor form, which contains an 80 aa mitochondrial targeting sequence (MTS) at the N-terminus. The full-length hFXN protein (amino acids 1-210) comprises the amino acid sequence of SEQ ID NO: 1.

Full-length hFXN hFXN₁₋₂₁₀
SEQ ID NO: 1
MWTLGRRAVAGLLASPSPAQAQTLTRVPRPAELAPLCGRRGLRTDIDATC
TPRRASSNQRGLNQIWNVKKQSVYLMNLRKSGTLGHPGSLDETTYERLAE
ETLDSLAEFFEDLADKPYTFEDYDVSFGSGVLTVKLGGDLGTYVINKQTP
NKQIWLSSPSSGPKRYDWTGKNWVYSHDGVSLHELLAAELTKALKTKLDL
SSLAYSGKDA

As the protein is imported into the mitochondrial matrix, it gets cleaved at amino acid 81, resulting in the mature form of FXN, which yields the mature 130 aa active FXN, with a predicted molecular weight of 14.2 kDa (SEQ ID NO: 1).

Mature hFXN hFXN₈₁₋₂₁₀ A
SEQ ID NO: 2
SGTLGHPGSLDETTYERLAEETLDSLAEFFEDLADKPYTFEDYDVSFGSG
VLTVKLGGDLGTYVINKQTPNKQIWLSSPSSGPKRYDWTGKNWVYSHDGV
SLHELLAAELTKALKTKLDLSSLAYSGKDA

The full-length hFXN (SEQ ID NO: 1) comprises mature hFXN (SEQ ID NO: 2) and a mitochondrial targeting sequence (MTS) having the amino acid sequence

(SEQ ID NO: 3)
MWTLGRRAVAGLLASPSPAQAQTLTRVPRPAELAPLCGRRGLRTDIDATC
TPRRASSNQRGLNQIWNVKKQSVYLMNLRK.

The fusion protein includes the HIV-TAT peptide (YGRKKRRQRRR) linked via a linker to the N-terminus of the full-length hFXN protein. The mechanism of action of the fusion protein relies on the cell-penetrating ability of the HIV-TAT peptide to deliver the fusion protein into cells and the subsequent processing into mature hFXN after translocation into the mitochondria. A particular fusion protein, CTI-1601, is described in U.S. Provisional Patent Application No. 62/880,073 and No. 62/891,029, the entire contents of each of which are hereby incorporated herein by reference.

CTI-1601 comprises the following amino acid sequence (224 amino acids):

(SEQ ID NO: 12)
MYGRKKRRQRRRGGMWTLGRRAVAGLLASPSPAQAQTLTRVPRPAELAPL
CGRRGLRTDIDATCTPRRASSNQRGLNQIWNVKKQSVYLMNLRKSGTLGH
PGSLDETTYERLAEETLDSLAEFFEDLADKPYTFEDYDVSFGSGVLTVKL
GGDLGTYVINKQTPNKQIWLSSPSSGPKRYDWTGKNWVYSHDGVSLHELL
AAELTKALKTKLDLSSLAYSGKDA.

### FXN Conditional Knockout (KO) Animals

A mouse model for FRDA (FXN-KO:MCK-Cre) established by the Jackson laboratory was used. In this model Fxn^{flox/null}::MCK-Cre mice harboring a Cre-conditional Frataxin allele, a frataxin global knockout allele and a cardiac/skeletal muscle-specific Cre recombinase transgene. The Fxn^{flox/null}::MCK-Cre mice (Stock No. 029720) develop progressive cardiomyopathy due to Frataxin protein deficiency in heart and skeletal muscle. Mutants exhibit peak body weight by 9 weeks of age and have a mean survival of 86+/−5 days of age. Cardiomyopathic phenotype is characterized by decreased heart rate and ejection fraction, as well as fractional shortening distinguishable from non-mutant littermates by approximately 7 weeks of age. Left ventricular mass is significantly increased compared to non-mutant littermates by 9 weeks of age.

### In Vivo Administration of the FXN Fusion Protein

Three groups of animals (eight animals in each group) were used in the present study, one control and two knockout FXN-KO:MCK-Cre. At 5 weeks of age, the FXN fusion protein at 10 mg/kg, or vehicle (50 mM NaOAc, 0.1 PEG) was administered to animals from each group, respectively. Administration of the drug was via sub-cutaneous injection at a volume of 10 mL/kg. The animals received test drug or vehicle every 48 hours until they reached 77 days of age. Twenty-four hours after the final dose (at eleven weeks), all animals were sacrificed, and perfused with PBS to clarify the tissues. Hearts were excised and preserved in an RNAse-free reagent compatible with preservation of tissues for further RNA analysis. One such reagent inactivates RNases and stabilizes RNA within tissues, for example RNA Later™.

### Cardiac Performance

Since conditional knock out mice have loss of FXN in the heart, cardiac performance by conscious ECG and anesthetized echocardiography was evaluated in all eight animals from each group before administration of the FXN fusion protein at 4 weeks of age, and after administration of the FXN fusion protein at 8 and 10 weeks of age.

### RNA Sequencing (RNASeq)

RNA from representatives of all groups (one control animal vehicle treated, two knock out vehicle treated animals, and two knockout animals treated with the FXN fusion protein) was isolated and prepared for sequencing. RNA Sequencing was performed using KAPA Stranded RNA-Seq Kit with RiboErase (HMR) Illumina® Platforms KR1151-v4.16. Roughly 100 million paired-end Illumina reads, 151 nt in length (before trimming), were sequenced from each sample. Adapter sequences were trimmed from FastQ files using cutadapt v1.2.1. Low-quality bases (Q<30) from the 3′ end of reads were removed and reads with more than 30% low-quality bases (Q<30) overall were filtered out. The remaining reads were aligned to the April 2018 Ensembl release of the mouse reference genome (GRCm38 v92 primary assembly) using RSEM v1.3.0 specifying STAR v2.5.3 as the aligner. Rsem was used to generate *.genes.results files for each sample.

### Friedreich's Ataxia (FDRA)-Derived Patient's Fibroblasts

FDRA patient's fibroblasts are referred to in the Results and Figures as FA GM03816 and FA 68. Cells were maintained in high glucose DMEM medium supplemented with 10% FBS and grown to confluence. Once achieving confluency, cells were kept for 24 hours without changing the medium, prior to RNA isolation.

### RNA Extraction

Upon reaching confluency, cells were rinsed with PBS buffer. Total RNA extraction was performed using RNeasy Mini Kit (Qiagen catalog number 74104) including the optional genomic DNA removal step, according to the protocol provided by the manufacturer. Total RNA concentration was measured in solution using a Beckman Coulter DU730 UV/Vis Spectrophotometer.

### Reverse Transcription (RT)-cDNA Synthesis

Reverse transcription was performed using 4 ug of total RNA in 30 uL reaction using the Superscript IV VILO Master Mix with ezDNase Kit (Invitrogen catalogue number 766500), according to the protocol provided by the manufacturer.

### Quantitative Real-Time Polymerase Chain Reaction (PCR)

Quantitative PCR, or real-time (RT) PCR, used herein interchangeably, was performed using the Quant Studio 5 automated system (Applied Biosystems). The reaction master mix was TaqMan Fast Advanced Master mix (ThermoFisher 4444557) and the plates were MicroAmp Fast 96-Well Reaction Plate (ThermoFisher 4346907). In general, each reaction (each well) consisted of: 10 uL Master Mix (20×)+0.33 uL Housekeeping Gene Primer/Probe (60×)+1 uL Target gene Primer/Probe (20×)+6.67 uL Nuclease Free H₂O+2 uL cDNA (approximately 25 ng). The PCR cycle included a 2-minute UNG (from the uracil-DNA glycosylases family, used for removal of uracil) incubation at 50° C., a 2-minute incubation at 95° C. for polymerase activation, and 40 PCR cycles of 1 second at 95° C. and 20 seconds at 60° C.

The PCR reaction comprised forward and reverse primers. By way of example, the forward primers are between 18 and 22 nucleotides long, and may comprise 15, 16, 17, 18, 19, 20, or 21 nucleotides identical to the target nucleic acid, the target nucleic acid being the sequence of any one of the FSGMs presented in Table 2, Table 4 and/or FIG. 3. The reverse primer may be complementary to the target nucleic acid. The reverse primer may also comprise a sequence complementary to an adaptor sequence.

Quantitative PCR (qPCR) of Housekeeping Genes

β-Actin transcript was used as the housekeeping gene as its level of expression was shown to be constant across FA-patient-derived fibroblasts (Disease Models & Mechanisms (2017) 10, 1353-1369 doi:10.1242/dmm.030536.) The primer-probe set (Hs01060665_g1) was purchased from ThermoFisher. The probe oligo was tagged with both a fluorescent dye (for example VIC dye, with an absorbance maximum of 538 nm and an emission maximum of 554 nm, thus emitting in the green-yellow part of the visible spectrum, or alternatively HEX dye) and a non-fluorescent quencher (NFQ-MGB Quencher).

### Quantitative PCR of FXN-Sensitive Genomic Markers (FXN Signature)

For the development of the method disclosed herein, target genes, herein referred to as FXN-sensitive genomic markers (FSGMs) were selected based on the RNASeq analysis of RNA from the hearts of FXN conditional knock out mice treated or non-treated with the FXN fusion protein. ThermoFisher qPCR primer-probe sets of the selected targets are shown in Table 1. Target gene probes were labeled with a fluorescent dye (Fluorescein amidite, FAM) along with a quencher (NFQ-MGB).

TABLE 1
Primer-probe sets

ABCE1 - Hs00759267_s1
ADAMTS1 - Hs00199608_m1
ALAS1 - Hs00167441_m1
APOLD1 - Hs00707371_S1
ATF3 - Hs00231069_m1
CH25H - Hs02379634_s1
CYR61 - Hs00155479_m1
CUL2 - Hs00180203_m1
CYCS - Hs01588974_g1
EGR1 - Hs00152928_m1
EGR2 - Hs00166165_M1
EGR3 - Hs00231780_m1
EIF1AX - Hs00796778_s1
hFXN - Hs00175940_m1
HIF1a - Hs00153153_m1
IGF1 - Hs01547656_m1
LAMP2 - Hs00903587_m1
Lars2 - Hs01118920_m1
MAOA - Hs00165140_m1
MKI67 - Hs00606991_m1
MPC1 - Hs00211484_m1
mt-ATP6 - Hs02596862_g1
mt-ATP8 - H202596863_g1
mt-CO2 - Hs02596865_g1
mt-CO3 - Hs02596866_g1
mt-ND1 - Hs02596873_s1
mt-ND2 - Hs02596874_g1
mt-ND3 - Hs02596875_s1
mt-ND4 - Hs02596876_g1
mt-RnR1 - Hs02596859_g1
mt-RnR2 - Hs02596860_s1
NR4A1 - Hs00374226_m1
PDE4a - Hs00183479_m1
PICALM - Hs00200318_m1
RAP2c - Hs00221801_m1
RnF13 - Hs00961508_g1
RPL10 - Hs01095478_g1
RPL24 - Hs02338570_gH
RPL26 - Hs00864008_m1
RPL32 - Hs00851655_g1
RPL38 - Hs01019601_g1
RPL39 - Hs04194816_g1
RPS15A - Hs03043791_m1
RPS23 - Hs01922548_s1
RPS27L - Hs00955038_g1
SLC25A25 - Hs01595834_g1
SLIRP - Hs00364015_m1
SMTN - Hs01022255_g1
UBE2D3 - Hs00704312_s1
YARS - Hs00169373_m1
ZNRF1 - Hs00936381_m1

In order to verify the validity of the PCR results, two tests were used as quality control: (1) linearity of the signal, which was established for each primer/probe by titrating the dCT (differential cycle threshold) as a function of cDNA concentration using normal HEK293 RNA; and (2) no quantifiable CT in a reverse-transcriptase (RT) minus control sample, in order to confirm that the signal was not due to contaminating genomic DNA (gDNA) in the RNA preparation.

Cycle threshold (CT) values were generated by the PCR apparatus. Each well was assigned two CT values, one for β-Actin and one for the target gene. The delta CT value was calculated by subtracting the β-Actin CT from the target gene CT for each well (target CT—β-Actin CT). The average of the baseline sample (i.e., untreated) ACT was calculated and used as the “normal” or “untreated” Baseline Sample. The Baseline Sample was subtracted from the “treated” sample delta CT. (Treated Sample ACT [minus] Baseline Sample ACT), resulting in the ΔΔCT for each “treated” sample. Fold-change for each individual sample was calculated by using the formula 2ΔΔCT. The replicate values were then averaged, and standard deviation was calculated as the error.

### Genomic Expression Following In Vivo Treatment

The hearts of FXN fusion protein-treated knock out (KO) mice or control mice were collected, and the RNA extracted for analysis. RNA sequencing as described above was used for obtaining the transcription expression profile triggered with or without treatment with the FXN fusion protein. Analysis of the differential expression of genes following treatment with the FXN fusion protein was performed as described below.

Differential expression (DE) analysis of the RNASeq results was performed with R version 3.44 and version 3.7 Bioconductor libraries. The non-adjusted “expected count” columns from rsem were imported with tximport and used as input for DEseq2. Tximport and DESeq2 were used with all default settings, except that genes with apparent lengths of 0 were reasserted to have lengths of 0.1 before running DESeq2. Two initial reports were compiled (data not shown) from the DESeq2 analyses: “all frataxin knockout samples versus all wild type samples”, and “all drug-treated samples versus all vehicle control samples”. Data in “drug-treated samples versus all vehicle control samples” report were sorted according to their adjusted p value (padj). Genes with a padj <0.005 were considered for further evaluation.

A cutoff for the base mean of 320 (read-out of the RNASeq analysis) was applied in the “frataxin knockout (KO) samples vs. all wild type (WT) samples” report, and genes below this threshold were not considered further if they were downregulated in “drug-treated samples vs. all vehicle-treated control samples”.

Genes meeting these criteria, i.e., genes whose expression was (i) above 320 in the “frataxin knockout vs. WT samples” and (ii) were either up or down regulated in “drug-treated vs. vehicle-treated knockout animals”; or genes whose expression was (i) below 320 in the “frataxin knockout vs. WT samples” and (ii) were only upregulated in “drug-treated vs. vehicle-treated knockout animals” were further restricted to genes for which the log 2FoldChange was greater than 0.584 or lower than −0.584, corresponding to approximately a 2-fold induction or repression, respectively.

Genes that met all the above-described criteria were taken as frataxin-sensitive genomic markers (FXN-induced signature) and used for generating FXN expression profile and examined for contrary regulation between the different treatments. Additional genes that fell slightly short of the criteria described above, but for which, upon further scrutiny, a strong rational existed, were included in the list of potential FSGMs as genes to be tested in additional models. For example, mt-CO2, which is up-regulated 3.21 fold in the FXN KO compared to the WT animal, and is downregulated 0.57 fold in CTI-1601 treated KO compared to vehicle treated, was included because it only narrowly missed the significance cutoff, other mt-DNA encoded Complex IV subunits did show up as being affected (mt-Co2 is expected to be similarly regulated since the gene is polycistronic), and one of the major protein levels regulated by LRPPRC, a significant hit, is mt-Co2. This progressive selection approach allowed the identification of genes that are contrary regulated by FXN gene ablation followed by FXN protein replacement, defining the replacement FXN expression profile. These genes reacted as sensitive to FXN, possibly being FXN target genes, and were considered bona fide markers of FXN replacement, as opposed to other genes, not contrary regulated, which are likely to be merely markers for changes in tissue remodeling or inflammation (data not shown).

Following in vivo treatment in mice with the FXN fusion protein, one hundred and two (102) genes presented significant differential expression, being either upregulated or downregulated when compared to control (Fold regulation in “KO vs. WT”=baseline Frataxin(−) signature), and these are detailed in Table 2. Most importantly, these genes were found to be contrary regulated in the Frataxin deficient mouse model upon treatment with the FXN fusion protein (Fold regulation in “drug (FXN fusion protein) vs. vehicle (Veh)”=replacement Frataxin signature). In other words, certain genes that showed upregulated expression in the absence of Frataxin had downregulated expression following treatment with the FXN fusion protein. Conversely, the reverse was also true, namely certain genes that showed downregulated expression in the absence of Frataxin had upregulated expression following treatment with the FXN fusion protein. This result was particularly surprising since Frataxin has never been described as a transcriptional regulator and therefore, regulation of downstream genes was not expected.

The Frataxin-sensitive genomic markers (FSGMs) presented in Table 2 may be grouped, for example, according to homology and/or function. For example, several mitochondrial genes which were induced or repressed in the knock out animals were shown to have their expression pattern reversed upon treatment with the FXN fusion protein. Mitochondrial gene transcripts CYR61, mt-ATP6, mt-ATP8, mt-CO2, mt-CO3, mt-ND1, mt-ND2, mt-ND3 and mt-ND4 were shown to be downregulated upon treatment with the FXN fusion protein, whereas mitochondrial gene transcripts mt-RNR1 and mt-RNR2 were upregulated, as can be seen in “FXN fusion protein vs. Veh” in Table 2. Expression of transcripts from the EGR family, EGR1, EGR2 and EGR3, or from the insulin growth factor family, IGF1, and LAMP2 were also downregulated following treatment with the FXN fusion protein. Similarly, SLIRP expression was downregulated upon treatment with the FXN fusion protein. A further group of markers that presented altered expression includes ADNP, AI480526, C230034O21RIK, CCDCl85B, CCDCl85C, CTCFL, NRTN, PDE4A, PHF1, RPL37RT, SLC26A10, SNORD17, SUV420H2, WNK2, YAM1 and ZNRF1; this set of markers showing upregulation following treatment with the FXN fusion protein.

The FSGMs presented in Table 2 can also be grouped, for example, according to whether they are secreted proteins. For example, as indicated in Table 2, CYR61, ADAMTS1, ASPN, FAM177A, IGF1, LOX, NRTN, SERPINE1, STC1, and THBS1 are all secreted proteins.

TABLE 2
Differential Expression of Genes Following Treatment With FXN
Fusion Protein (FXN-Induced Signature)

| | | | FXN fusion |
|---|---|---|---|
| Gene | Secreted | | protein vs. |
| Symbol | protein? | KO vs. WT | Veh |
| Abce1 | N | 2.54 | 0.54 |
| Adamts1 | Y | 2.45 | 0.32 |
| Adnp | N | 0.47 | 2.06 |
| AI480526 | N | 0.23 | 3.68 |
| Apold1 | N | 7.14 | 0.26 |
| Arc | N | 6.98 | 0.22 |
| Aspn | Y | 4.44 | 0.37 |
| Atf3 | N | 2.49 | 0.31 |
| Bicd1 | N | 2.30 | 0.48 |
| Btg2 | N | 3.46 | 0.54 |
| C230034O21Rik | | 0.38 | 3.18 |
| Calm2 | N | 2.11 | 0.49 |
| Capza1 | N | 2.71 | 0.54 |
| Ccdc85b | N | 0.47 | 2.47 |
| Ccdc85c | N | 0.41 | 2.30 |
| Chm | N | 2.31 | 0.49 |
| Cops2 | N | 3.18 | 0.47 |
| Cript | N | 2.06 | 0.45 |
| Ctcfl | N | 0.31 | 3.67 |
| Ctss | N | 2.47 | 0.47 |
| Cul2 | N | 2.81 | 0.52 |
| Cycs | N | 2.02 | 0.38 |
| Cyr61 | Y | 5.71 | 0.44 |
| D130020L05Rik | | 0.21 | 5.74 |
| Dclk1 | N | 2.20 | 0.33 |
| Dcun1d1 | N | 2.71 | 0.45 |
| Dfna5 | N | 2.62 | 0.35 |
| Dio2 | N | 2.08 | 0.38 |
| Dnajb9 | N | 2.33 | 0.48 |
| Dsel | N | 2.55 | 0.43 |
| Dynlt3 | N | 2.33 | 0.44 |
| Egr1 | N | 7.21 | 0.42 |
| Egr2 | N | 19.26 | 0.04 |
| tgr3 | N | 11.78 | 0.18 |
| mt-CO2 | N | 3.21 | 0.57 |
| Eif1a | N | 2.57 | 0.43 |
| Emp1 | N | 4.88 | 0.43 |
| Fam177a | Y | 2.64 | 0.46 |
| Gmfb | N | 2.60 | 0.52 |
| Hist1h4n | N | 2.43 | 0.23 |
| Igf1 | Y | 3.78 | 0.36 |
| Kctd12b | N | 4.24 | 0.49 |
| Lamp2 | N | 2.00 | 0.50 |
| Lamtor5 | N | 2.12 | 0.43 |
| Lox | Y | 5.23 | 0.46 |
| Lypla1 | N | 2.18 | 0.46 |
| Lysmd3 | N | 2.10 | 0.46 |
| Maoa | N | 14.07 | 0.44 |
| Mki67 | N | 5.16 | 0.40 |
| Mob4 | N | 2.52 | 0.38 |
| Mpeg1 | N | 4.85 | 0.39 |
| Mt2 | N | 5.47 | 0.50 |
| mt-Atp6 | N | 7.44 | 0.23 |
| mt-Atp8 | N | 10.16 | 0.14 |
| mt-Co3 | N | 4.73 | 0.33 |
| nit-Nd1 | N | 4.53 | 0.33 |
| mt-Nd2 | N | 3.90 | 0.27 |
| mt-Nd3 | N | 5.58 | 0.23 |
| mt-Nd4 | N | 3.03 | 0.40 |
| mt-Rnr1 | N | 0.05 | 69.55 |
| mt-Rnr2 | N | 0.06 | 22.51 |
| Nr4a1 | N | 2.57 | 0.18 |
| Nrtn | Y | 0.45 | 2.25 |
| Orc4 | N | 2.25 | 0.42 |
| Pde4a | N | 0.45 | 1.39 |
| Pde4b | N | 1.21 | 0.45 |
| Phf1 | N | 0.45 | 2.08 |
| Psma3 | N | 2.07 | 0.47 |
| Ptgs2 | N | 5.12 | 0.07 |
| Ptp4a1 | N | 2.61 | 0.51 |
| Ptprc | N | 2.09 | 0.33 |
| Rap1b | N | 2.62 | 0.47 |
| Rap2c | N | 3.29 | 0.48 |
| Rnf13 | N | 2.18 | 0.50 |
| Rnf2 | N | 2.12 | 0.41 |
| Rpl10 | N | 2.18 | 0.34 |
| Rpl24 | N | 2.92 | 0.49 |
| Rpl26 | N | 2.21 | 0.45 |
| Rpl32 | N | 2.60 | 0.46 |
| Rpl37rt | | 0.23 | 7.79 |
| Rpl38 | N | 2.48 | 0.48 |
| Rpl39 | N | 3.21 | 0.39 |
| Rps15a | N | 2.53 | 0.41 |
| Rps27l | N | 3.49 | 0.40 |
| Rtn4 | N | 4.61 | 0.45 |
| Serpine1 | Y | 4.68 | 0.37 |
| Slc26a10 | N | 0.44 | 3.41 |
| Slirp | N | 2.86 | 0.24 |
| Snord17 | N | 0.23 | 2.94 |
| Spry4 | N | 2.83 | 0.46 |
| Stc1 | Y | 4.83 | 0.23 |
| Suv420h2 | N | 0.39 | 2.27 |
| Thbs1 | Y | 3.40 | 0.35 |
| Tmem126a | N | 2.08 | 0.43 |
| Top2a | N | 4.92 | 0.27 |
| Ube2d3 | N | 2.94 | 0.44 |
| Vbp1 | N | 2.40 | 0.45 |
| Wnk2 | N | 0.44 | 2.29 |
| Yam1 | N | 0.12 | 9.77 |
| Yars | N | 2.33 | 1.45 |
| Zfp758 | | 2.37 | 0.33 |
| Znf41-ps | | 9.66 | 0.15 |
| Znrf1 | N | 0.49 | 2.26 |

In Table 2, the values contained in the columns identified as “knock out (KO) vs. wild-type (WT)” (column 1) and “FXN-fusion vs. vehicle” (column 2) indicate whether the FSGM is increased or decreased with efficacious FXN replacement therapy.

More specifically, for a given FSGM, if the value in column 2 (FXN-fusion vs. vehicle) is less than 1.0 and the value in column 1 (KO vs. WT) is greater than 1.0, then the FSGM level is both increased in the FXN-depleted condition (as compared to wild type), and decreased when efficacious FXN replacement therapy is administered, and is therefore contrary regulated (e.g., CYR61).

Conversely, for a given FSGM, if the value in column 2 (FXN-fusion vs. vehicle) is greater than 1.0 and the value in column 1 (KO vs. WT) is less than 1.0, then the FSGM level is both decreased in the FXN-depleted condition (as compared to wild type), and increased when efficacious FXN replacement therapy is administered, and is therefore contrary regulated (e.g., YAM1).

### Example 2. String Analysis of FSGMs

This example describes a String analysis of the FSGMs presented in Table 2, showing that the protein products of the FSGMs are at least partially biologically connected, as a group.

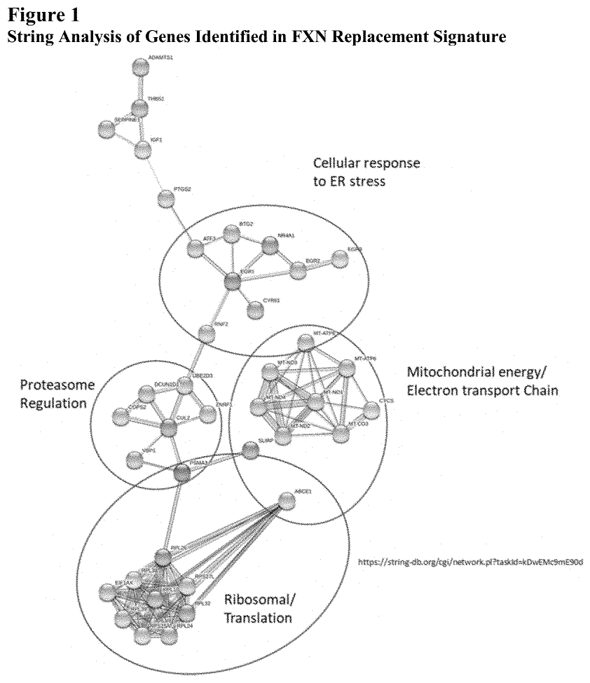

String analysis using the String database (string-db.org; Szklarczyk et al. (2015) doi: 10.1093/nar/gkv1277 and references therein) was performed using 85 protein products of the FXN-sensitive genomic markers described in Table 2. The string analysis is presented in FIG. 1. String analysis represents an example of known and/or predicted protein interactions according to their function. The parameters used for generating the clusters in the string analysis were: nodes=85; edges=97; average node degree=2.28; average local clustering coefficient=0.345; expected number of edges=35; PPI enrichment p-value <1.0e-16. The minimum required interaction score was 0.700 (high confidence). Disconnected nodes in the network were hidden. The following parameters were used as active interaction sources: Textmining, Experiments, Databases, Co-expression, Neighborhood, Gene Fusion and Co-occurrence. The partition used allowed one marker to be part of more than one cluster. For simplification purposes, only some of the clusters are clearly visible from FIG. 1, while others are not visible in the Figure, but these were also listed herein below.

Under the parameters specified above, the following are an example of some of the clusters and their respective markers obtained by string analysis:
Response to Ribosome Depletion or Endoplasmic Reticulum (ER) Stress—NR4A1, PTP4A1, ATF3, BTG2, EGR1, EGR2, EGR3, CYR61, ABCE1;
Mitochondrial Energy Production—MT-ND1, MT-ND2, MT-ND3, MT-ND4, MT-CO3, MT-ATP6, MT-ATP8, CYCS;
Regulation of the Proteasome and Unfolded Protein Response—COPS2, VBP1, PSMA3, SLIRP, CUL2, DCUN1D1, UBE2D3, ZNRF1, RNF2, LAMP2;
Ribosomal Function—RPS15A, EIF1AX, RPL24, RPL32, RPL26, RPL10, RPL39, RPL38, RPS27L; ABCE1,
Respiratory Chain—MT-ND1, MT-ND2, MT-ND3, MT-ND4, MT-CO3, CYCS;
Cardiac Muscle Development—NR4A1, EGR1, EGR3, ADAMTS1, THBS1, SERPINE1, IGF1, PTGS2, CYR61;
Macromolecule Catabolism—PSMA3, CUL2, UBE2D3, ZNRF1, RPS15A, RPL24, RPL32, RPL26, RPL10, RPL39, RPL38;
Translational Initiation—ABCE1, RPS15A, EIF1AX, RPL24, RPL32, RPL26, RPL10, RPL39, RPL38;
Mitochondrial Components—MT-ND1, MT-ND2, MT-ND3, MT-ND4, MT-CO3, MT-ATP6, MT-ATP8, CYCS, TMEM-126A, MAOA, ABCE1;
Oxidative Phosphorylation—MT-ND1, MT-ND2, MT-ND3, MT-ND4, MT-CO3, MT-ATP6, MT-ATP8;
Negative Regulation of Macromolecule Metabolic Process—ABCE1, RPL26, RPL38, RPL10, RPL32, RPS15A, RPL24, RPL39, SLIRP, COPS2, DCUN1D1, RNF2, EGR1, BTG2, ATF3, PTGS2, IGF1, SERPINE1, THBS1;
Regulation of apoptotic process—RPL26, THBS1, SERPINE1, IGF1, PTGS2, RPL10, RPS27L, CYCS, ATF3, BTG2, EGR1, EGR3, CYR61.

Other clusters that resulted from the string analysis with a false discovery rate of up to 0.003 were: protein targeting to membrane, SRP-dependent co-translational protein targeting to membrane, translation, nuclear-transcribed mRNA catabolic process, primary metabolic process, cellular metabolic process, protein targeting, peptide metabolic process, negative regulation of cellular process, cellular macromolecule metabolic process, organic substance metabolic process, regulation of cellular protein metabolic process, regulation of protein metabolic process, skeletal muscle cell differentiation, respiratory electron transport chain, metabolic process, cytoplasmic translation, regulation of cell cycle, cellular component organization of biogenesis, mitochondrial electron transport, NADH to ubiquinone, angiogenesis, regulation of macromolecule metabolic process, nucleobase-containing compound catabolic process, establishment of protein localization to organelle, cellular process, cellular macromolecule catabolic process, purine ribonucleoside monophosphate metabolic process, macromolecule catabolic process, response to stress, and response to oxygen.

The clusters of proteins based on the string analysis indicate that the FSGMs protein products have more potential interactions among themselves than would be expected for a random set of proteins of similar size, drawn from the genome. Such an enrichment indicates that the protein products of the FSGMs are at least partially biologically connected, as a group.

### Example 3: Selection of Potential FXN Target Genes Following In Vitro Treatment

The identification of genes that are contrary regulated by FXN gene ablation followed by FXN protein replacement in vivo suggested that the gene expression changes induced by FXN replacement treatment could be used as indicative of treatment effectiveness in patients treated with FXN replacement therapy. Based on this premise, a baseline FXN-induced signature was tested in two in vitro human cell models: Friedreich's Ataxia (FDRA)-derived fibroblasts and in

Frataxin Protein and mRNA Expression in Human Cell Models

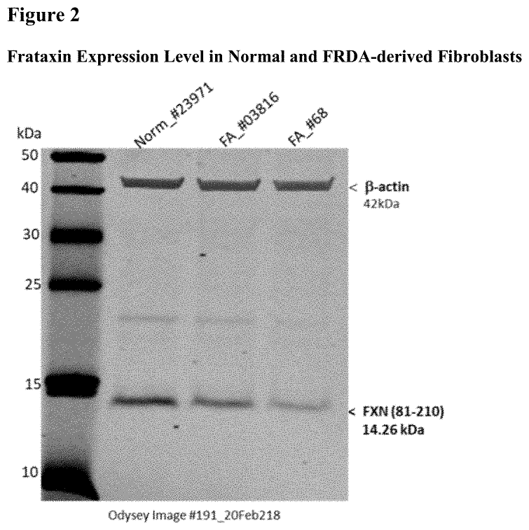

Frataxin protein and mRNA expression were examined in the FDRA-derived fibroblasts. Frataxin protein expression was shown and quantified from a Western blot gel, while Frataxin mRNA expression was quantified by qRT-PCR. Results are presented in FIG. 2 and Table 3.

FIG. 2 shows Frataxin protein detection in control GM23971 cells and FDRA-derived fibroblasts FA GM03816 and FA 68. β-actin signal was used for frataxin signal normalization when doing quantification of protein expression. Frataxin protein levels in the control GM23971 cells were considered as 100%, in relation to Frataxin protein in FDRA-derived fibroblasts FA GM03816 and FA 68 which were 64% and 31% from control, respectively. Frataxin mRNA quantification showed similar results, with FA GM03816 and FA 68 having about 66% and 32% of mRNA expression when compared to control cells (Table 3).

TABLE 3
Expression of frataxin protein and mRNA in FRDA-derived fibroblast
cells (FA) compared to normal fibroblasts

| | | | | | % FXN | % FXN |
|---|---|---|---|---|---|---|
| | | | | | protein | mRNA |
| | | | | | (relative to | (relative to |
| | GAA₁/ | FXN | β-actin | FXN/ | Normal | Normal |
| Cell Type | GAA₂ | signal | signal | β-actin | cells) | cells) |
| GM07522 | normal | NA | NA | NA | NA | 117 |
| (normal) | | | | | | (+/−33) |
| GM23971 | normal | 85.9 | 8400 | 0.0102 | 100 | 83 |
| (normal) | | | | | | (+/−15) |
| GM03816 | 330/380 | 55.3 | 8450 | 0.0065 | 64 | 66 |
| (FA) | | | | | | (+/−9) |
| 68 (FA) | 570/1200 | 24.4 | 7620 | 0.0032 | 31 | 32 |
| | | | | | | (+/−0.7) |

### Development of Frataxin-Induced Genetic Signature in Cell Models

Development of a Baseline FXN(−) Expression Profile:

One example of a baseline FXN deficient (FXN(−)) expression profile was identified and is shown in FIG. 3, in the comparison between normal cells, N-GM07522 and N-GM23971, and Frataxin-depleted cells from FDRA-derived fibroblasts, FA-GM03816, FA-GM04078, FA-4654, FA-68 (not shown), FA-4675, and FA-4194 (not shown). Altered expression was identified for ABCE1, APOLD1, ATF3, CYR61, CUL2, CYCs, EGR1, EGR2, EGR3, EiFIAX, IGF1, LAMP2, MAOA, NR4a1, PDE4A, RnF13, RPL10, RPL24, RPL26, RPL32, RPL38, RPL39, RPS15A, RPS23, RPS27L, SLIRP, UBE2D3,YARS, ZNRF1, and mitochondrial transcripts mt-ATP6, mt-ATP8, mt-CO2, mt-CO3, mt-ND1, mt-ND2, mt-ND3, mt-ND4, mt-RNR1 and mt-RNR2.

### Effect of Frataxin Administration in FDRA-Derived Fibroblasts:

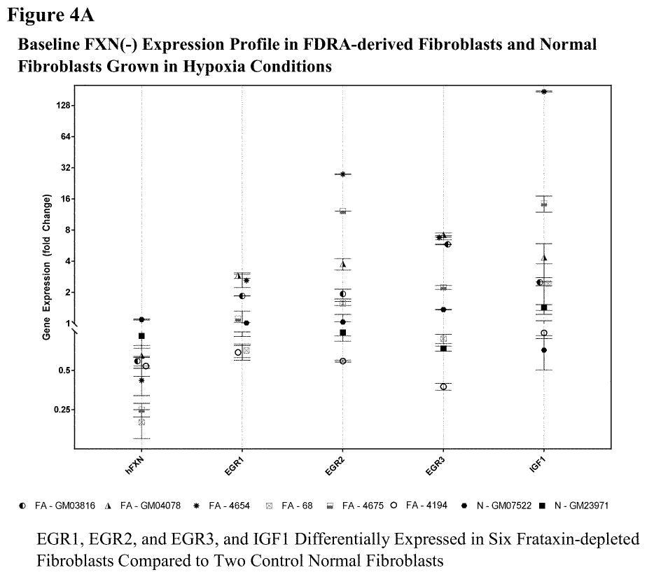

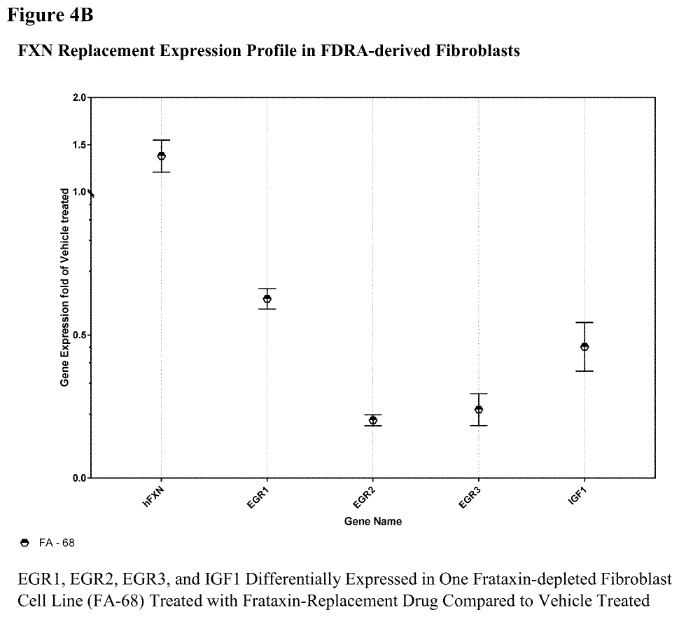

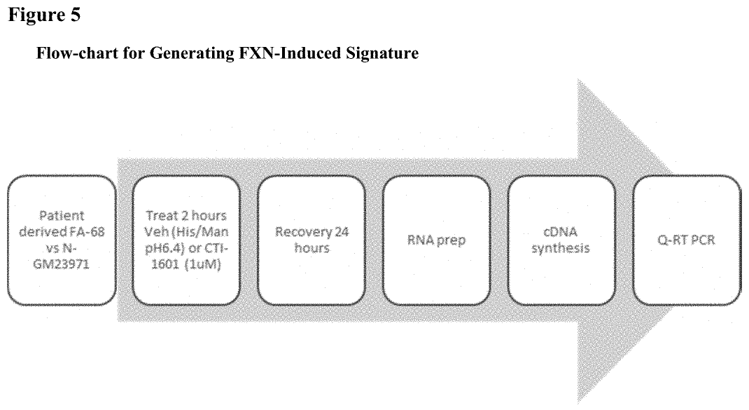

Gene expression analysis of FRDA patients-derived fibroblasts show that several transcription factors and secreted proteins are overall upregulated in the patients-derived fibroblasts compared to normal fibroblasts (FIG. 3 and FIG. 4A). In order to evaluate the effect of frataxin replacement in FDRA, FA-derived fibroblasts (lineage FA-68) were treated with the FXN fusion protein or vehicle-treated, RNA collected and processed for PCR analysis. The results are presented in FIG. 4B and represent fold of gene expression in cells treated with the FXN fusion protein relative to vehicle-treated cells. hFXN expression is shown as an internal control. FIG. 4B provides an exemplary FXN replacement expression profile, represented by the downregulation of EGR1, EGR2, EGR3 and IGF1 expression which was detected in the frataxin-depleted cell line upon treatment with the FXN fusion protein. A schematic of the procedure is presented in FIG. 5.

### Example 4: Detection of FXN Signature in FXN Fusion Protein-Treated Patient Sample

A sample of blood, buccal or muscle cells is collected from a FDRA patient before and after treatment with Frataxin replacement therapy, for example an FXN fusion protein. The two samples (pre- and post-treatment) are processed for RNA extraction and RT-PCR is performed for FSGMs presented in Table 2, Table 4 and/or FIG. 3. Analysis of the results of the RT-PCR of the two samples will show which transcripts were altered, upregulated or downregulated after treatment, and will provide an indication of the effectiveness of the FXN replacement therapy. The presence of contrary regulation of FSGMs when comparing their expression before and after FXN replacement treatment will be the indication of an effective treatment. For example, the detection of downregulation of at least one of CYR61, EGR1, EGR2, EGR3 and/or IGF1 post-treatment would indicate that the FXN replacement therapy has been effective. In contrast if no contrary regulation is detected in at least one FSGM comparing before and after treatment would indicate treatment failure. Similarly, obtaining the feature vectors from the FXN expression profiles in pre- and post-treatment samples, and comparing these with the deficient FXN feature vector and the FXN replacement feature vector described herein above will provide indication on the effectiveness of the FXN replacement therapy. As a result of the FXN signature obtained for the patient's sample, a new FXN replacement therapy dosage regime may be adopted, by increasing or decreasing the dosage of FXN replacement therapy being administered to the patient.

### Example 5. Generation of an In Vitro Cell Model for Frataxin (FXN) Knockdown (KD)

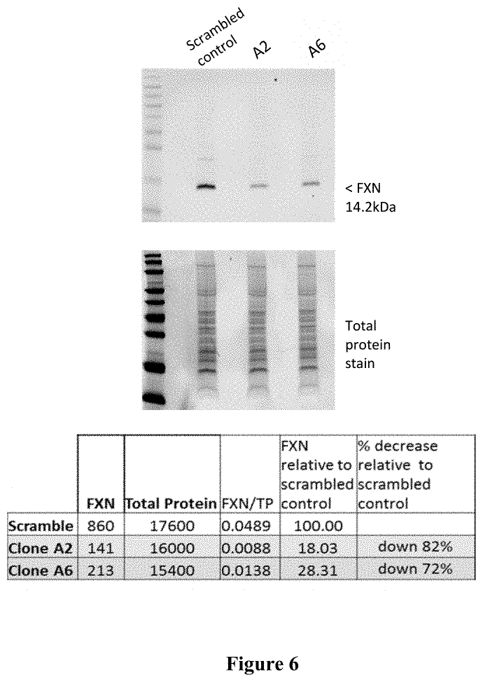

HEK293 cells were transfected with a KD-hFXN shRNA construct in order to repress frataxin mRNA and protein expression in the cells. A scrambled control shRNA construct that was not specific to FXN was used as a control. As shown in FIG. 6, expression of FXN protein in the KD-FXN clones A2 and A6 was significantly reduced when compared to the scrambled control. The table in FIG. 6 shows the results of protein quantitation in the Western blot, expressed as the amount of FXN in FXN KD cells relative to the amount of FXN in the scrambled control cells. The results shown in the table in FIG. 6 indicate that the amount of FXN protein in the KD-FXN clones A2 and A6 is reduced by 82% and 72%, respectively, as compared to the scrambled control.

### Example 6. Effect of Treatment with an FXN Fusion Protein on CYR61 Protein Expression in hFXN-KD Cells

The goal of this experiment was to determine changes in the levels of CYR61 in response to treatment with an FXN fusion protein, CTI-1601, in scrambledcontrol and hFXN-KD cell lines produced as described in Example 5. To this end, scrambledcontrol and hFXN-KD (clone A6) cells were seeded on a 6-well tissue culture plate pre-coated with 1% fibronectin solution at a density of 150,000 cells/well in 1 mL of treatment media (DMEM, 5% heat inactivated FBS, 20 mM glycerol and 20 mM HEPES). After 1 hour, the cells in each well were treated with different concentrations of CTI-1601. Specifically, 50 μL of a serial dilution of CTI-1601 (20 μM, 10 μM, 5 μM, 2.5 μM, and 1.25 μM, as well as 0 μM control) in formulation buffer (20 mM histidine, 250 mM sucrose, 0.05% polysorbate 20, pH 5.8) was added to each well, and the plates were incubated for 3 hours in an incubator. Subsequently, 1 mL of Complete Media (10% FBS, DMEM containing antibiotics) was added to each well, and the plates were incubated for 21 hours. This cycle was repeated 3 times on days 1, 2, and 3, and then the plates were incubated for an additional day. On day 5, pictures of the plates were taken, 1 mL of media was harvested, supplemented with 10 μL HALT protease inhibitor and frozen at −80° C. for further analysis.

The amount of CYR61 protein secreted into the cell media was measured using the CYR61 ELISA (R&D Biosystems—CDYR10) according to the manufacturer's protocol. The media from scrambled control and hFXN-KD cells was diluted 1:2 before analysis.

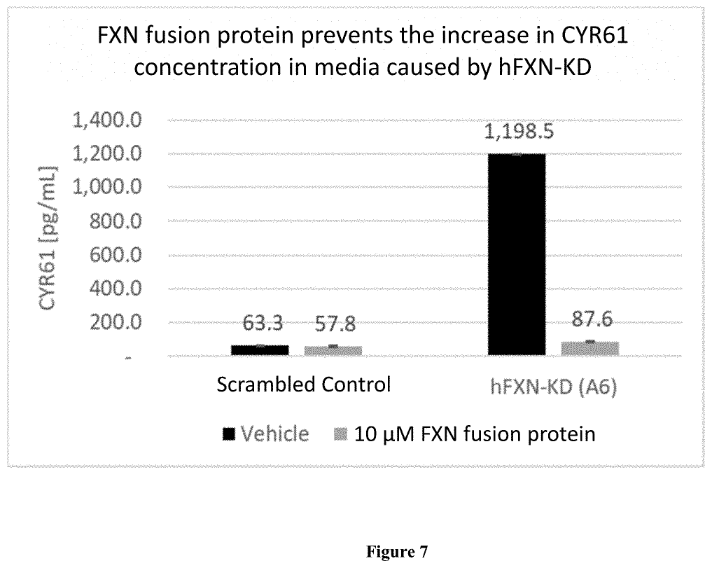

The results of the ELISA analysis for hFXN-KD cells are presented in FIG. 7. The results indicate that there is a relatively low level of CYR61 protein (about 63.3 pg/mL) in the media from the scrambled control cells, and this level is not affected by treatment with 10 μM CTI-1601. In contrast, consistent with mRNA data, the level of CYR61 protein secreted in the media from the hFXN-KD cells is significantly higher (about 1,198.5 pg/mL) as compared to the level of CYR61 protein in the media from the scrambled control cells. In addition, FIG. 7 also indicates that the levels of the secreted CYR61 protein from the hFXN-KD cells are significantly decreased to control levels (about 87.6 pg/mL) after treatment with 10 μM CTI-1601.

These results demonstrate once again that CYR61 is contrary modulated by FXN knock down followed by FXN protein replacement, and that, in addition to changes in gene expression levels, detection of secreted CYR61 protein can serve as a marker of FXN protein replacement.

### Example 7. Transfection of hFXN into hFXN-KD Cells Causes a Decrease in the Amount of Secreted CYR61 Protein

The goal of this experiment was to determine if transfection of hFXN-KD cells with hFXN can reverse the mitochondrial impairment in these cells as measured by the amount of secreted CYR61 protein. To this end, hFXN-KD and scrambled control HEK293 cells described in Example 5 were transfected with an empty pCDNA3 vector (+V) or with an expression vector for full-length hFXN: pCDNA3-hFXN (+hFXN) using Fugene-6 reagent according to the manufacturer's instructions and incubated for 48 hours. The transfected cells were incubated for additional 48 hours. After the second 48-hour incubation, 1 mL of media was removed and 10 μL of HALT protease inhibitor was added to the aliquot. The amount of CYR61 protein in the media was measured using Simple Step CYR61 Elisa from ABCAM™ (ab238267) according to the manufacturer's protocol. For the measurement, the media from scrambled control and hFXN-KD cells was diluted 1/10. Data was plotted using Graphpad Prism Bar graph with Standard Deviation as error bars.

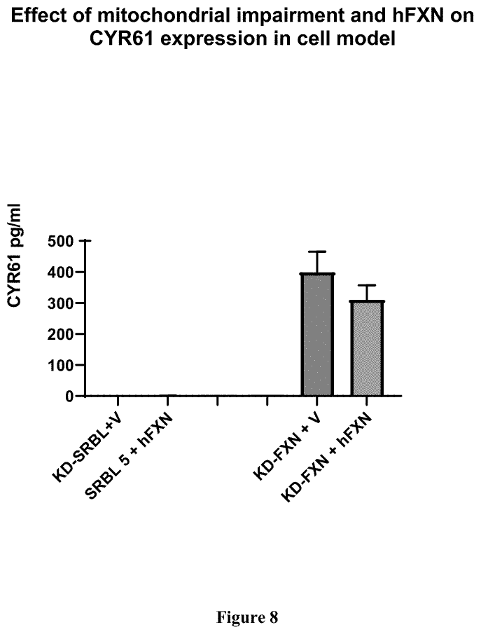

The results of this experiment are shown in FIG. 8, which is a bar graph showing the amount of CYR61 protein in the media from the scrambled control cells transfected with an empty vector (KD-SRBL+V); scrambled control cells transfected with hFXN (SRBL 5+hFXN); hFXN-KD cells transfected with an empty vector (KD-FXN+V); and hFXN-KD cells transfected with hFXN (KD-FXN+hFXN). FIG. 8 indicates that the scrambled control cells do not secrete detectable levels of CYR61 protein either in the absence or in the presence of exogenously expressed hFXN. There is abundant CYR61 protein secreted from the hFXN-KD cells transfected with an empty vector, and the amount of secreted CYR61 protein is decreased by the transient expression of hFXN in these cells.

These results demonstrate that CYR61 is contrary modulated by FXN knock down followed by replacement FXN protein expression driven through a nucleic acid mediated expression, and further demonstrate that detection of secreted CYR61 protein can serve as a marker of FXN protein replacement.

### Example 8. Level of CYR61 is Increased in FXN Knockout Mouse Embryonic Stem Cells

The goal of this experiment was to determine if levels of the secreted CYR61 protein are altered in mouse embryonic stem (ES) B9 cells in which the FXN gene is deleted (knocked out).

### Production of FNX-Knockout Mouse Cell Line

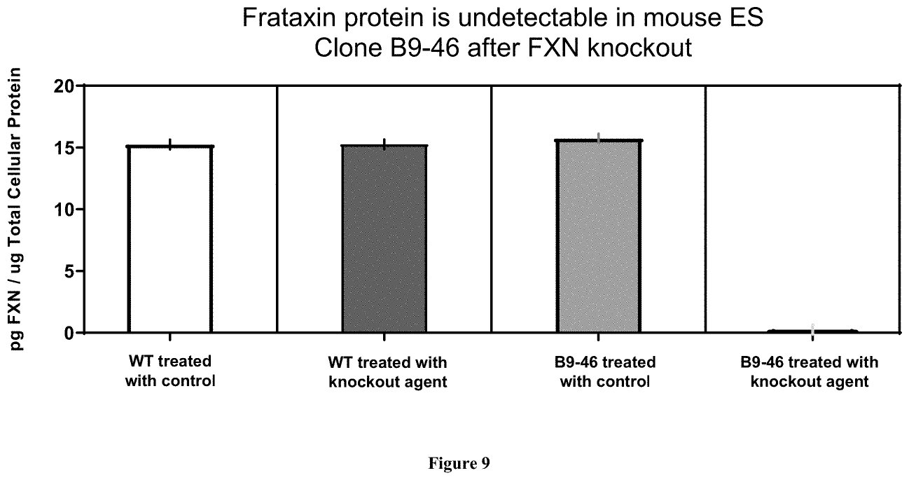

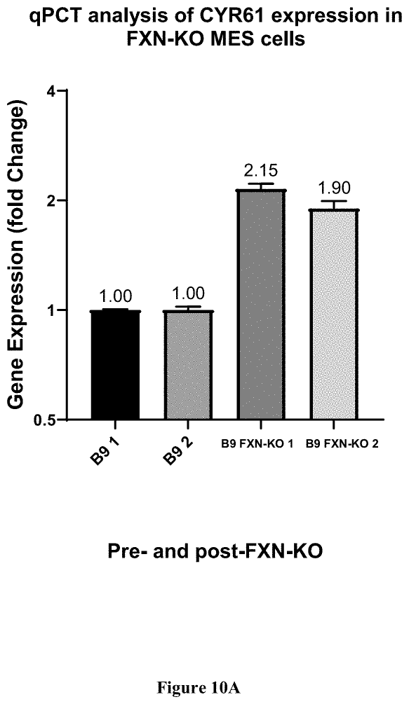

A mouse embryonic stem cell line deficient in FXN was produced. Specifically, a homozygous mouse ES clone B9-46 was produced as a result of this experiment, which may be induced to knock out both alleles of the FXN gene. FIG. 9 is a bar graph showing the amount of FXN protein per total cellular protein in the WT mouse ES clone and the homozygous mouse ES clone B9-46 which has been treated with control or an agent to induce the FXN knockout (knockout agent). The amount of mouse FXN protein was measured using Mouse FXN Elisa kit (Abcam ab199078) according to manufacturer's protocol. FIG. 9 indicates that treatment with an agent to induce FXN knockout resulted in the elimination of the FXN protein in B9-46 cells. No decrease in the levels of the FXN protein was observed in the WT cells or control-treated B9-46 cells.

Measurement of CYR61 Gene Expression (mRNA and Protein)

Mouse B9 cells were treated with a control agent or an agent to induce knockdown of the FXN gene. To measure the amount of CYR61 gene expression, RNA was extracted from the B9 mouse cells and the amount of CYR61 mRNA was measured using qPCR as previously described. The TaqMan Primers™ used for the qPCR analysis were purchased from ThermoFisher and β-actin was used as a housekeeping gene (β-actin VIC PL: Hs01060665_g1; CYR61: Hs00155479_m1). Two biological replicates were analyzed for each of agent and control treatment. To measure the amount of secreted CYR61 in cell media, 1 mL of cell media was harvested, supplemented with 10 μL HALT protease inhibitor and frozen at −80° C. for further analysis. The amount of the secreted CYR61 protein was measured using ELISA as previously described.

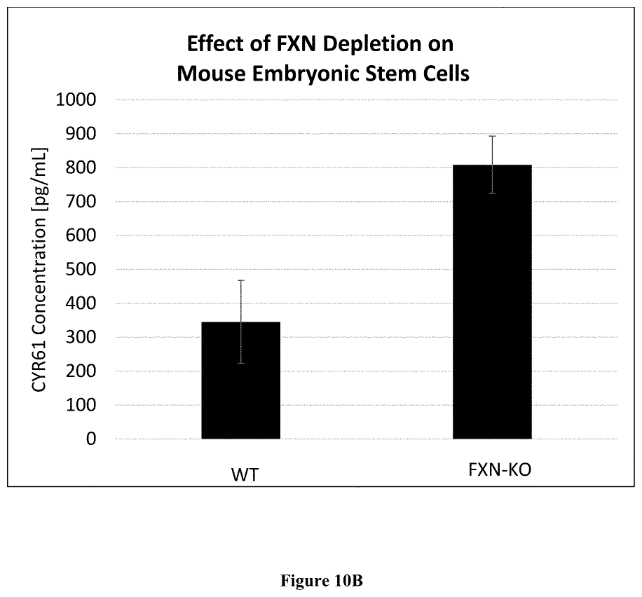

The results of the CYR61 gene expression analysis are presented in FIG. 10, panel A. The results indicate that knockout of the FXN gene in the B9 cells results in an approximate 2-fold increase in the expression of CYR61 mRNA. The results of the measurement of the levels of secreted CYR61 protein are presented in FIG. 10, panel B. The results indicate that knockout of the FXN gene in the B9 cells results in an approximate 2-fold increase in the amount of CYR61 protein levels in the cell media.

Descriptions of embodiments of the disclosure in the present application are provided by way of example and are not intended to limit the scope of the disclosure. The described embodiments comprise different features, not all of which are required in all embodiments. Some embodiments utilize only some of the features or possible combinations of the features. Variations of embodiments of the disclosure that are described, and embodiments comprising different combinations of features noted in the described embodiments, will occur to persons of the art. The scope of an embodiment of the disclosure is limited only by the claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

1. A method for evaluating effectiveness of frataxin (FXN) replacement therapy, the method comprising:
(a) determining an FXN replacement expression profile for one or more FXN-sensitive genomic markers (FSGMs) in a sample from an FXN deficient patient following treatment with FXN replacement therapy;
(b) comparing the patient FXN replacement expression profile with a baseline FXN(−) expression profile; and
(c) using the comparison to determine effectiveness of the FXN replacement therapy;
wherein the one or more FSGMs are any one or more markers defined in Table 2, Table 4 and/or FIG. 3.

2. The method according to claim 1, further comprising determining a baseline FXN(−) expression profile for one or more FXN-sensitive genomic markers (FSGMs) in a sample from a patient exhibiting FXN deficiency prior to FXN replacement therapy.

3. The method according to claim 2, wherein the one or more FSGMs comprise at least one or any combination of more than one of a gene encoding a secreted protein, a mitochondrial gene, a EGR-family gene, insulin-like gene, ribosome depletion response gene, mitochondrial energy production gene, proteasome regulation gene, ribosomal function gene, respiratory chain gene, cardiac muscle development gene, macromolecule catabolism gene, a translational initiation gene, mitochondrial components gene, oxidative phosphorylation gene, negative regulation of macromolecule metabolic process gene, or regulation of apoptotic process gene, or a protein encoded by any of these genes.

4. The method of claim 1, wherein the one or more FSGMs comprise a secreted protein.

5. The method of claim 1, wherein the one or more FSGMs comprise one or more of:
(a) CYR61, ADAMTS1, ASPN, FAM177A, IGF1, LOX, NRTN, SERPINE1, STC1, and THBS1;
(b) NR4A1, PTP4A1, ATF3, BTG2, EGR1, EGR2, EGR3, CYR61, and ABCE1;
(c) EGR1, EGR2, EGR3 and IGF1;
(d) MT-ND1, MT-ND2, MT-ND3, MT-ND4, MT-CO3, MT-ATP6, MT-ATP8, and CYCS;
(e) OPS2, VBP1, PSMA3, SLIRP, CUL2, DCUN1D1, UBE2D3, ZNRF1, RNF2, and LAMP2;
(f) RPS15A, EIF1AX, RPL24, RPL32, RPL26, RPL10, RPL39, RPL38, RPS27L, and ABCE1;
(g) MT-ND1, MT-ND2, MT-ND3, MT-ND4, MT-CO3, and CYCS;
(h) NR4A1, EGR1, EGR3, ADAMTS1, THBS1, SERPINE1, IGF1, PTGS2, and CYR61;
(i) PSMA3, CUL2, UBE2D3, ZNRF1, RPS15A, RPL24, RPL32, RPL26, RPL10, RPL39, and RPL38;
(j) ABCE1, RPS15A, EIF1AX, RPL24, RPL32, RPL26, RPL10, RPL39, and RPL38;
(k) MT-ND1, MT-ND2, MT-ND3, MT-ND4, MT-CO3, MT-ATP6, MT-ATP8, CYCS, TMEM-126A, MAOA, and ABCE1;
(l) MT-ND1, MT-ND2, MT-ND3, MT-ND4, MT-CO3, MT-ATP6, and MT-ATP8;
(m) ABCE1, RPL26, RPL38, RPL10, RPL32, RPS15A, RPL24, RPL39, SLIRP, COPS2, DCUN1D1, RNF2, EGR1, BTG2, ATF3, PTGS2, IGF1, SERPINE1, and THBS1; or
(n) RPL26, THBS1, SERPINE1, IGF1, PTGS2, RPL10, RPS27L, CYCS, ATF3, BTG2, EGR1, EGR3, and CYR61.

6. The method of claim 1, wherein the one or more FSGMs comprise CYR61.

7-19. (canceled)

20. The method according to claim 1, wherein the one or more FSGMs are upregulated following treatment with FXN replacement therapy.

21. The method according to claim 20, wherein the one or more FSGMs that are upregulated following treatment with FXN replacement therapy are mt-RNR1, mt-RNR2, ADNP, AI480526, C230034O21RIK, CCDCl85B, CCDCl85C, CTCFL, NRTN, PDE4A, PHF1, RPL37RT, SLC26A10, SNORD17, SUV420H2, WNK2, YAM1 or ZNRF1.

22. The method according to claim 1, wherein the one or more FSGMs are downregulated following treatment with FXN replacement therapy.

23. The method according to claim 22, wherein the one or more FSGMs that are downregulated following treatment with FXN replacement therapy are CYR61, mt-ATP6, mt-ATP8, mt-CO2, mt-CO3, mt-ND1, mt-ND2, mt-ND3 and mt-ND4, EGR1, EGR2, EGR3, IGF1, LAMP2, or SLIRP.

24. The method according to claim 1, wherein determining an FXN expression profile for FSGMs comprises determining an FXN feature vector of values indicative of expression of the FSGMs.

25. The method according to claim 24, wherein using the comparison to determine effectiveness of the FXN replacement therapy comprises determining first and second FXN feature vectors for the patient FXN replacement expression profile and the baseline FXN(−) expression profile respectively and determining a distance between the feature vectors.

26. The method according to claim 25, wherein determining the distance between the feature vectors comprises determining a scalar product of the first and second feature vectors.

27. The method according to claim 25, further comprising determining a third feature vector for a normal FXN expression profile for the FSGMs for a healthy subject.

28. The method according to claim 27, further comprising determining a distance between the second and third feature vectors.

29. The method according to claim 28, further comprising determining a distance between the first and third feature vectors, and normalizing the distance between the first and third feature vectors to the distance between the second and third feature vectors.

30. The method according to claim 29, further comprising using the normalized distance to determine effectiveness of the FXN replacement therapy.

31. The method according to claim 1, wherein
the expression profile is determined by any one of sequencing, hybridization or amplification of the sample RNA; or
wherein the expression profile is determined by HPLC/UV-Vis spectroscopy, enzymatic analysis, mass spectrometry, NMR, immunoassay, ELISA, or any combination thereof.

32. (canceled)

33. The method according to claim 1, further comprising modifying treatment with the FXN replacement therapy when the FXN replacement therapy is indicated as being ineffective.

34. The method according to claim 1, wherein the patient is suffering from Freidrich's Ataxia (FRDA).

35. The method according to claim 1, further comprising obtaining a biological sample from a patient exhibiting FXN deficiency.

36. (canceled)

37. The method of claim 1, wherein the FXN replacement therapy comprises treatment with an FXN fusion protein.

38. The method of claim 1, wherein the FXN replacement therapy comprises treatment with CTI-1601.

39-43. (canceled)

44. A method of detecting one or more frataxin-sensitive genomic markers (FSGMs) in a biological sample from a patient suffering from a frataxin (FXN) deficiency by contacting the biological sample, or a portion thereof, with one or more detection reagents specific for detection of one or more FSGMs, wherein the one or more FSGM comprises one or more FSGMs selected from Table 2, Table 4 and/or FIG. 3.

45-50. (canceled)

51. A method of treatment of a mitochondrial disease, the method comprising:
providing a sample from a subject suffering from FXN deficiency,
determining an FXN expression profile in the sample for one or more FXN-sensitive genomic markers (FSGMs),
comparing the FXN expression profile of the sample with at least one other expression profile selected from the group consisting of normal FXN expression profile for one or more FSGMs, baseline FXN(−) expression profile for one or more FSGMs, and an FXN replacement expression profile for one or more FSGMs,
classifying the sample FXN expression profile as corresponding to a normal FXN expression profile, baseline FXN(−) expression profile or an FXN replacement expression profile, initiating, increasing or decreasing the dosage of FXN replacement therapy to be administered to the subject based on the classification of the sample FXN expression profile.

52-68. (canceled)

69. A composition for determining the expression profile of FSGMs, the composition comprising reagents for the detection of at least one or more FSGMs described in Table 2, Table 4 and/or FIG. 3.

70-72. (canceled)

73. A kit for detecting one or more frataxin-sensitive genomic marker (FSGM) in a biological sample from a subject exhibiting frataxin (FXN) deficiency or being treated for FXN deficiency, comprising one or more reagents for measuring the level of the one or more FSGM in the biological sample from the subject, wherein the one or more FSGM comprises one or more FSGMs selected from Table 2, Table 4 and/or FIG. 3, and a set of instructions for measuring the level of the FSGM.

74-77. (canceled)

78. A panel for use in a method of monitoring or evaluating the efficacy of frataxin (FXN) replacement therapy, the panel comprising one or more detection reagents, wherein each detection reagent is specific for the detection of one or more frataxin-sensitive genomic marker (FSGM), wherein the one or more FSGM comprises one or more markers selected from Table 2, Table 4 and/or FIG. 3.

79-83. (canceled)
